(19) United States
(12) Patent Application Publication
(10) Pub. No.: US 2021/0069174 A1
(43) Pub. Date: Mar. 11, 2021

(54) COMPOSITIONS AND METHODS FOR TREATMENT OF CYSTIC FIBROSIS

(71) Applicant: Vertex Pharmaceuticals Incorporated, Boston, MA (US)

(72) Inventors: Cathy Chu, Cambridge, MA (US); Eleni Dokou, Cambridge, MA (US); Eric L. Haseltine, Melrose, MA (US); Samuel Moskowitz, Waban, MA (US); Kirk A. Overhoff, Lynn, MA (US); Sarah Robertson, Somerville, MA (US); David Waltz, Waban, MA (US)

(21) Appl. No.: 16/625,028

(22) PCT Filed: Jun. 29, 2018
(86) PCT No.: PCT/US2018/040427

§ 371 Date: Dec. 20, 2019

Related U.S. Application Data
(60) Provisional application No. 62/528,072, filed on Jul. 1, 2017.
(60) Provisional application No. 62/533,396, filed on Jul. 17, 2017.
(60) Provisional application No. 62/633,171, filed on Feb. 21, 2018.

(51) Int. Cl. A61K 31/47; A61K 9/20; A61K 31/4439; A61K 31/404; A61K 9/16; A61P 11/00

(52) U.S. Cl. CPC A61K 31/47; A61K 9/20; A61K 31/4439; A61K 31/404; A61P 11/00; A61K 9/2013; A61K 9/2095; A61K 9/16; A61K 9/2054

(57) ABSTRACT

Compositions comprising Compound I of the formula (I) and methods of treating cystic fibrosis comprising administering Compound I. Compositions comprising a pharmaceutically acceptable salt of Compound I and methods of treating cystic fibrosis comprising administering a pharmaceutically acceptable salt of Compound I.

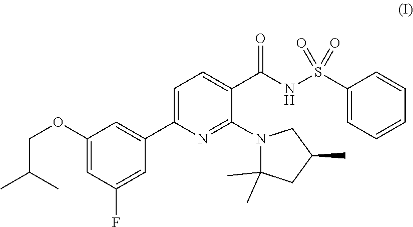 [SMILES: CC(C)COc1cc(F)cc(-c2ccc(C(=O)NS(=O)(=O)c3ccccc3)c(N3C[C@@H](C)CC3(C)C)n2)c1]

The present application claims the benefit of priority of U.S. Provisional Application No. 62/528,072, filed Jul. 1, 2017, U.S. Provisional Application No. 62/533,396, filed Jul. 17, 2017, and U.S. Provisional Application No. 62/633,171, filed Feb. 21, 2018, the entire contents of which are incorporated herein by reference.

Disclosed herein is a modulator of Cystic Fibrosis Transmembrane Conductance Regulator (CFTR), pharmaceutical compositions containing the modulator, methods of treatment of cystic fibrosis, and a process for making the modulator.

Cystic fibrosis (CF) is a recessive genetic disease that affects approximately 70,000 children and adults worldwide. Despite progress in the treatment of CF, there is no cure.

In patients with CF, mutations in CFTR endogenously expressed in respiratory epithelia lead to reduced apical anion secretion causing an imbalance in ion and fluid transport. The resulting decrease in anion transport contributes to enhanced mucus accumulation in the lung and accompanying microbial infections that ultimately cause death in CF patients. In addition to respiratory disease, CF patients typically suffer from gastrointestinal problems and pancreatic insufficiency that, if left untreated, result in death. In addition, the majority of males with cystic fibrosis are infertile, and fertility is reduced among females with cystic fibrosis.

Sequence analysis of the CFTR gene has revealed a variety of disease causing mutations (Cutting, G. R. et al. (1990) Nature 346:366-369; Dean, M. et al. (1990) Cell 61:863:870; and Kerem, B-S. et al. (1989) Science 245:1073-1080; Kerem, B-S et al. (1990) Proc. Natl. Acad. Sci. USA 87:8447-8451). To date, greater than 2000 mutations in the CF gene have been identified; currently, the CFTR2 database contains information on only 322 of these identified mutations, with sufficient evidence to define 281 mutations as disease causing. The most prevalent disease-causing mutation is a deletion of phenylalanine at position 508 of the CFTR amino acid sequence, and is commonly referred to as the F508del mutation. This mutation occurs in approximately 70% of the cases of cystic fibrosis and is associated with severe disease.

The deletion of residue 508 in CFTR prevents the nascent protein from folding correctly. This results in the inability of the mutant protein to exit the endoplasmic reticulum (ER) and traffic to the plasma membrane. As a result, the number of CFTR channels for anion transport present in the membrane is far less than observed in cells expressing wild-type CFTR, i.e., CFTR having no mutations. In addition to impaired trafficking, the mutation results in defective channel gating. Together, the reduced number of channels in the membrane and the defective gating lead to reduced anion and fluid transport across epithelia. (Quinton, P. M. (1990), FASEB J. 4: 2709-2727). The channels that are defective because of the F508del mutation are still functional, albeit less functional than wild-type CFTR channels. (Dalemans et al. (1991), Nature Lond. 354: 526-528; Pasyk and Foskett (1995), J. Cell. Biochem. 270: 12347-50). In addition to F508del, other disease causing mutations in CFTR that result in defective trafficking, synthesis, and/or channel gating could be up- or down-regulated to alter anion secretion and modify disease progression and/or severity.

CFTR is a cAMP/ATP-mediated anion channel that is expressed in a variety of cell types, including absorptive and secretory epithelia cells, where it regulates anion flux across the membrane, as well as the activity of other ion channels and proteins. In epithelial cells, normal functioning of CFTR is critical for the maintenance of electrolyte transport throughout the body, including respiratory and digestive tissue. CFTR is composed of approximately 1480 amino acids that encode a protein which is made up of a tandem repeat of transmembrane domains, each containing six transmembrane helices and a nucleotide binding domain. The two transmembrane domains are linked by a large, polar, regulatory (R)-domain with multiple phosphorylation sites that regulate channel activity and cellular trafficking.

Chloride transport takes place by the coordinated activity of ENaC and CFTR present on the apical membrane and the Nat⁺-K⁺-ATPase pump and Cl⁻ channels expressed on the basolateral surface of the cell. Secondary active transport of chloride from the luminal side leads to the accumulation of intracellular chloride, which can then passively leave the cell via Cl⁻ channels, resulting in a vectorial transport. Arrangement of Na⁺/2Cl⁻/K⁺ co-transporter, Na⁺-K⁺-ATPase pump and the basolateral membrane K⁺ channels on the basolateral surface and CFTR on the luminal side coordinate the secretion of chloride via CFTR on the luminal side. Because water is probably never actively transported itself, its flow across epithelia depends on tiny transepithelial osmotic gradients generated by the bulk flow of sodium and chloride.

Accordingly, there is a need for novel treatments of CFTR mediated diseases.

Disclosed herein is Compound I and pharmaceutically acceptable salts thereof. Compound I can be depicted as having the following structure:

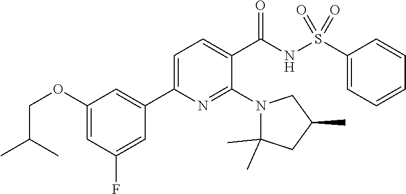 [SMILES: CC(C)COc1cc(F)cc(-c2ccc(C(=O)NS(=O)(=O)c3ccccc3)c(N3C[C@@H](C)CC3(C)C)n2)c1]

A chemical name for Compound I is N-(benzenesulfonyl)-6-(3-fluoro-5-isobutoxy-phenyl)-2-[(4S)-2,2,4-trimethylpyrrolidin-1-yl]pyridine-3-carboxamide. PCT Publication No. WO 2016/057572, incorporated herein by reference, discloses Compound I, a method of making Compound I, and that Compound I is a CFTR modulator with an EC₃₀ of <3 μM.

Disclosed herein are pharmaceutical compositions wherein the properties of one therapeutic agent are improved by the presence of two therapeutic agents, kits, and methods of treatment thereof. In some embodiments, the disclosure features pharmaceutical compositions comprising N-(benzenesulfonyl)-6-(3-fluoro-5-isobutoxy-phenyl)-2-[(4S)-2,2,4-trimethylpyrrolidin-1-yl]pyridine-3-carboxamide (Compound I), (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropanecarboxamide (Compound II), and N-[2,4-bis(1,1-dimethylethyl)-5-hydroxyphenyl]-1,4-dihydro-4-oxoquinoline-3-carboxamide (Compound III), wherein the composition has improved properties.

Also disclosed herein is a solid dispersion of N-(benzenesulfonyl)-6-(3-fluoro-5-isobutoxy-phenyl)-2-[(4S)-2,2,4-trimethylpyrrolidin-1-yl]pyridine-3-carboxamide (Compound I) in a polymer. In some embodiments, the solid dispersion is prepared by spray drying, and is referred to as a spray-dried dispersion (SDD). In some embodiments, the spray dried dispersion has a Tg of from 80° C. to 180° C. In some embodiments, Compound I in the spray dried dispersion is substantially amorphous.

Also disclosed are methods of treating the CFTR-mediated disease cystic fibrosis comprising administering N-(benzenesulfonyl)-6-(3-fluoro-5-isobutoxy-phenyl)-2-[(4S)-2,2,4-trimethylpyrrolidin-1-yl]pyridine-3-carboxamide (Compound I), (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropanecarboxamide (Compound II), and N-[2,4-bis(1,1-dimethylethyl)-5-hydroxyphenyl]-1,4-dihydro-4-oxoquinoline-3-carboxamide (Compound III), optionally as part of at least one pharmaceutical composition comprising at least one additional component, to a patient in need thereof.

### BRIEF DESCRIPTION OF THE DRAWINGS

FIG. 1 is a representative list of CFTR genetic mutations.

FIG. 2: Comparison of co-blend vs. co-spray SDDs dissolution in FeSSIF.

### Definitions

As used herein, “CFTR” means cystic fibrosis transmembrane conductance regulator.

As used herein, “mutations” can refer to mutations in the CFTR gene or the CFTR protein. A “CFTR gene mutation” refers to a mutation in the CFTR gene, and a “CFTR protein mutation” refers to a mutation in the CFTR protein. A genetic defect or mutation, or a change in the nucleotides in a gene in general results in a mutation in the CFTR protein translated from that gene, or a frame shift(s).

The term “F508del” refers to a mutant CFTR protein which is lacking the amino acid phenylalanine at position 508.

As used herein, a patient who is “homozygous” for a particular gene mutation has the same mutation on each allele.

As used herein, a patient who is “heterozygous” for a particular gene mutation has this mutation on one allele, and a different mutation on the other allele.

As used herein, the term “modulator” refers to a compound that increases the activity of a biological compound such as a protein. For example, a CFTR modulator is a compound that increases the activity of CFTR. The increase in activity resulting from a CFTR modulator includes but is not limited to compounds that correct, potentiate, stabilize and/or amplify CFTR.

As used herein, the term “CFTR corrector” refers to a compound that facilitates the processing and trafficking of CFTR to increase the amount of CFTR at the cell surface. Compounds I and II disclosed herein are CFTR correctors.

As used herein, the term “CFTR potentiator” refers to a compound that increases the channel activity of CFTR protein located at the cell surface, resulting in enhanced ion transport. Compound III disclosed herein is a CFTR potentiator.

As used herein, the term “active pharmaceutical ingredient” or “therapeutic agent” (“API”) refers to a biologically active compound.

As used herein, the term “pharmaceutically acceptable salt” refers to a salt form of a compound of this disclosure wherein the salt is nontoxic. Pharmaceutically acceptable salts of the compounds of this disclosure include those derived from suitable inorganic and organic acids and bases. Pharmaceutically acceptable salts are well known in the art. For example, S. M. Berge, et al. describes pharmaceutically acceptable salts in detail in J. Pharmaceutical Sciences, 1977, 66, 1-19.

As used herein, the term “amorphous” refers to a solid material having no long range order in the position of its molecules. Amorphous solids are generally supercooled liquids in which the molecules are arranged in a random manner so that there is no well-defined arrangement, e.g., molecular packing, and no long range order. Amorphous solids are generally isotropic, i.e. exhibit similar properties in all directions and do not have definite melting points. For example, an amorphous material is a solid material having no sharp characteristic crystalline peak(s) in its X-ray power diffraction (XRPD) pattern (i.e., is not crystalline as determined by XRPD). Instead, one or several broad peaks (e.g., halos) appear in its XRPD pattern. Broad peaks are characteristic of an amorphous solid. See, US 2004/0006237 for a comparison of XRPDs of an amorphous material and crystalline material.

In some embodiments, a solid material may comprise an amorphous compound, and the material may, for example, be characterized by a lack of sharp characteristic crystalline peak(s) in its XRPD spectrum (i.e. the material is not crystalline, but is amorphous, as determined by XRPD). Instead, one or several broad peaks (e.g. halos) may appear in the XRPD pattern of the material. See US 2004/0006237 for a comparison of XRPDs of an amorphous material and crystalline material. A solid material, comprising an amorphous compound, may be characterized by, for example, a wider temperature range for the melting of the solid material, as compared to the range for the melting of a pure crystalline solid. Other techniques, such as, for example, Raman spectroscopy, infrared spectroscopy, and solid state NMR may be used to characterize crystalline or amorphous forms.

In some embodiments, a solid material may comprise a mixture of crystalline solids and amorphous solids. A solid material prepared to comprise an amorphous compound may also, for example, contain up to 30% of a crystalline solid. In some embodiments, a solid material prepared to comprise an amorphous compound may also, for example, contain up to 25%, 20%, 15%, 10%, 5%, or 2% of a crystalline solid. In embodiments wherein the solid material contains a mixture of crystalline solids and amorphous solids, the characterizing data, such as XRPD, may contain indicators of both crystalline and amorphous solids. As used herein, the term “substantially amorphous” refers to a solid material having little or no long range order in the position of its molecules. For example, substantially amorphous materials have less than 15% crystallinity (e.g., less than 10% crystallinity or less than 5% crystallinity). It is also noted that the term ‘substantially amorphous’ includes the descriptor, ‘amorphous’, which refers to materials having no (0%) crystallinity.

As used herein, the term “dispersion” refers to a disperse system in which one substance, the dispersed phase, is distributed, in discrete units, throughout a second substance (the continuous phase or vehicle). The size of the dispersed phase can vary considerably (e.g. colloidal particles of nanometer dimension, to multiple microns in size). In general, the dispersed phases can be solids, liquids, or gases. In the case of a solid dispersion, the dispersed and continuous phases are both solids. In pharmaceutical applications, a solid dispersion can include a crystalline drug (dispersed phase) in an amorphous polymer (continuous phase); or alternatively, an amorphous drug (dispersed phase) in an amorphous polymer (continuous phase). In some embodiments, a solid dispersion includes the polymer constituting the dispersed phase, and the drug constitute the continuous phase. Or, a solid dispersion includes the drug constituting the dispersed phase, and the polymer constituting the continuous phase.

The terms “patient” and “subject” are used interchangeably and refer to an animal including humans.

The terms “effective dose” and “effective amount” are used interchangeably herein and refer to that amount of a compound that produces the desired effect for which it is administered (e.g., improvement in CF or a symptom of CF, or lessening the severity of CF or a symptom of CF). The exact amount of an effective dose will depend on the purpose of the treatment, and will be ascertainable by one skilled in the art using known techniques (see, e.g., Lloyd (1999) The Art, Science and Technology of Pharmaceutical Compounding).

As used herein, the terms “treatment,” “treating,” and the like generally mean the improvement of CF or its symptoms or lessening the severity of CF or its symptoms in a subject. “Treatment,” as used herein, includes, but is not limited to, the following: increased growth of the subject, increased weight gain, reduction of mucus in the lungs, improved pancreatic and/or liver function, reduction of chest infections, and/or reductions in coughing or shortness of breath. Improvements in or lessening the severity of any of these symptoms can be readily assessed according to standard methods and techniques known in the art.

As used herein, the term “in combination with,” when referring to two or more compounds, agents, or additional active pharmaceutical ingredients, means the administration of two or more compounds, agents, or active pharmaceutical ingredients to the patient prior to, concurrent with, or subsequent to each other.

The term “approximately”, when used in connection with doses, amounts, or weight percent of ingredients of a composition or a dosage form, includes the value of a specified dose, amount, or weight percent or a range of the dose, amount, or weight percent that is recognized by one of ordinary skill in the art to provide a pharmacological effect equivalent to that obtained from the specified dose, amount, or weight percent.

One of ordinary skill in the art would recognize that, when an amount of “a compound or a pharmaceutically acceptable salt thereof” is disclosed, the amount of the pharmaceutically acceptable salt form of the compound is the amount equivalent to the concentration of the free base of the compound. It is noted that the disclosed amounts of the compounds or their pharmaceutically acceptable salts thereof herein are based upon their free base form. For example, “100 mg of Compound I or its pharmaceutically acceptable salt” includes 100 mg of Compound I and a concentration of a pharmaceutically acceptable salt of Compound I equivalent to 100 mg of Compound I.

A. Solid Dispersions

In some embodiments, the disclosure provides a solid dispersion comprising Compound I or a pharmaceutically acceptable salt thereof.

In some embodiments, the disclosure provides a spray dried dispersion comprising Compound I or a pharmaceutically acceptable salt thereof.

In some embodiments, the solid dispersion comprises at least one compound chosen from Compound I and pharmaceutically acceptable salts thereof and further comprises one or more additional APIs. In some embodiments, such additional APIs are selected from at least one compound chosen from Compound II, Compound III, and pharmaceutically acceptable salts of any of the foregoing.

In some embodiments, the spray dried dispersion comprises comprises at least one compound chosen from Compound I and pharmaceutically acceptable salts thereof and further comprises one or more additional APIs. In some embodiments, such additional APIs are selected from at least one compound chosen from Compound II, Compound III, and pharmaceutically acceptable salts of any of the foregpong.

In some embodiments, the solid dispersions and the spray dried dispersions comprise a plurality of particles having a mean particle diameter of 5 to 100 microns. In some embodiments, the solid dispersion and the spray dried dispersion comprises a plurality of particles having a mean particle diameter of 5 to 30 microns. In some embodiments, the solid dispersion and the spray dried dispersion comprises a plurality of particles having a mean particle diameter of 15 microns.

In some embodiments, the solid dispersions and the spray dried dispersions of the disclosure comprises substantially amorphous Compound I. In some embodiments, the solid dispersion is a spray dried dispersion, wherein the spray dried dispersion is substantially amorphous.

In some embodiments, the solid dispersions and the spray dried dispersions of the disclosure can comprise other excipients, such as polymers and/or surfactants. Any suitable polymers and surfactants known in the art can be used in the disclosure. Certain exemplary polymers and surfactants are as described below.

In some embodiments, the solid dispersions and the spray dried dispersions of the disclosure comprise a polymer.

In some embodiments, the solid dispersions and the spray dried dispersions of the disclosure are substantially free of polymer.

Methods of Preparing Solid Dispersions

Solid dispersions of any one of Compounds I, II and III may be prepared by any suitable method know in the art, e.g., spray drying, lyophilizing, hot melting, or cyrogrounding/cryomilling techniques. For example, see WO2015/160787. Typically such spray drying, lyophilizing, hot melting or cyrogrounding/cryomilling techniques generates an amorphous form of API (e.g., Compound I, II or III).

Spray drying is a process that converts a liquid feed to a dried particulate form. Optionally, a secondary drying process such as fluidized bed drying or vacuum drying may be used to reduce residual solvents to pharmaceutically acceptable levels. Typically, spray drying involves contacting a highly dispersed liquid suspension or solution, and a sufficient volume of hot gas to produce evaporation and drying of the liquid droplets. The preparation to be spray dried can be any solution, coarse suspension, slurry, colloidal dispersion, or paste that may be atomized using the selected spray drying apparatus. In one procedure, the preparation is sprayed into a current of warm filtered gas that evaporates the solvent and conveys the dried product to a collector (e.g. a cyclone). The spent gas is then exhausted with the solvent, or alternatively the spent air is sent to a condenser to capture and potentially recycle the solvent. Commercially available types of apparatus may be used to conduct the spray drying. For example, commercial spray dryers are manufactured by Buchi Ltd. And Niro (e.g., the PSD line of spray driers manufactured by Niro) (see, US 2004/0105820; US 2003/0144257).

Techniques and methods for spray drying may be found in Perry's Chemical Engineering Handbook, 6th Ed., R. H. Perry, D. W. Green & J. O. Maloney, eds.), McGraw-Hill book co. (1984); and Marshall “Atomization and Spray-Drying” 50, Chem. Eng. Prog. Monogr. Series 2 (1954).

Removal of the solvent may require a subsequent drying step, such as tray drying, fluid bed drying, vacuum drying, microwave drying, rotary drum drying or biconical vacuum drying.

In some embodiments, the solid dispersions and the spray dried dispersions of the disclosure are fluid bed dried.

In one process, the solvent includes a volatile solvent, for example a solvent having a boiling point of less than 100° C. In some embodiments, the solvent includes a mixture of solvents, for example a mixture of volatile solvents or a mixture of volatile and non-volatile solvents. Where mixtures of solvents are used, the mixture can include one or more non-volatile solvents, for example, where the non-volatile solvent is present in the mixture at less than 15%, e.g., less than 12%, less than 10%, less than 8%, less than 5%, less than 3%, or less than 2%.

In some processes, solvents are those solvents where the API(s) (e.g., Compound I, Compound II, and/or Compound III) has solubilities of at least 10 mg/ml, (e.g., at least 15 mg/ml, 20 mg/ml, 25 mg/ml, 30 mg/ml, 35 mg/ml, 40 mg/ml, 45 mg/ml, 50 mg/ml, or greater). In other processes, solvents include those solvents where the API(s) (e.g., Compound I, Compound II, and/or Compound III) has a solubility of at least 20 mg/ml.

Exemplary solvents that could be tested include acetone, cyclohexane, dichloromethane or methylene chloride (DCM), N,N-dimethylacetamide (DMA), N,N-dimethylformamide (DMF), 1,3-dimethyl-2-imidazolidinone (DMI), dimethyl sulfoxide (DMSO), dioxane, ethyl acetate, ethyl ether, glacial acetic acid (HAc), methyl ethyl ketone (MEK), N-methyl-2-pyrrolidinone (NMP), methyl tert-butyl ether (MTBE), tetrahydrofuran (THF), pentane, acetonitrile, methanol, ethanol, isopropyl alcohol, isopropyl acetate, and toluene. Exemplary co-solvents include DCM/methanol, acetone/DMSO, acetone/DMF, acetone/water, MEK/water, THF/water, dioxane/water. In a two solvent system, the solvents can be present in of from 0.1% to 99.9% w/w. In some embodiments, water is a co-solvent with acetone where water is present from 0.1% to 15%, for example 9% to 11%, e.g., 10%. In some embodiments, water is a co-solvent with MEK where water is present from 0.1% to 15%, for example 9% to 11%, e.g., 10%. In some embodiments the solvent system includes three solvents. Certain exemplary solvents include those described above, for example, MEK, DCM, water, methanol, IPA, and mixtures thereof.

The particle size and the temperature drying range may be modified to prepare an optimal solid dispersion. As would be appreciated by skilled practitioners, a small particle size would lead to improved solvent removal. Applicants have found however, that smaller particles can lead to fluffy particles that, under some circumstances do not provide optimal solid dispersions for downstream processing such as tableting.

A solid dispersion (e.g., a spray dried dispersion) of the present embodiment may optionally include a surfactant. A surfactant or surfactant mixture would generally decrease the interfacial tension between the solid dispersion and an aqueous medium. An appropriate surfactant or surfactant mixture may also enhance aqueous solubility and bioavailability of the API(s) (e.g., Compound I, Compound II, and/or Compound III) from a solid dispersion. The surfactants for use in connection with the disclosure include, but are not limited to, sorbitan fatty acid esters (e.g., Spans®), polyoxyethylene sorbitan fatty acid esters (e.g., Tweens®), sodium lauryl sulfate (SLS), sodium dodecylbenzene sulfonate (SDBS) dioctyl sodium sulfosuccinate (Docusate sodium), dioxycholic acid sodium salt (DOSS), Sorbitan Monostearate, Sorbitan Tristearate, hexadecyltrimethyl ammonium bromide (HTAB), Sodium N-lauroylsarcosine, Sodium Oleate, Sodium Myristate, Sodium Stearate, Sodium Palmitate, Gelucire 44/14, ethylenediamine tetraacetic acid (EDTA), Vitamin E d-alpha tocopheryl polyethylene glycol 1000 succinate (TPGS), Lecithin, MW 677-692, Glutanic acid monosodium monohydrate, Labrasol, PEG 8 caprylic/capric glycerides, Transcutol, diethylene glycol monoethyl ether, Solutol HS-15, polyethylene glycol/hydroxystearate, Taurocholic Acid, Pluronic F68, Pluronic F108, and Pluronic F127 (or any other polyoxyethylene-polyoxypropylene co-polymers (Pluronics®) or saturated polyglycolized glycerides (Gelucirs®)). Specific example of such surfactants that may be used in connection with this disclosure include, but are not limited to, Span 65, Span 25, Tween 20, Capryol 90, Pluronic F108, sodium lauryl sulfate (SLS), Vitamin E TPGS, pluronics and copolymers.

In some embodiments, SLS is used as a surfactant in the disclosure.

The amount of the surfactant (e.g., SLS) relative to the total weight of the solid dispersion may be between 0.1-15% w/w. In some embodiments, it is from 0.5% to 10%, such as from 0.5 to 5%, for example, 0.5 to 4%, 0.5 to 3%, 0.5 to 2%, 0.5 to 1%, or 0.5%.

In some embodiments, the amount of the surfactant relative to the total weight of the solid dispersion is at least 0.1%, such as at least 0.5%. In these embodiments, the surfactant would be present in an amount of no more than 15%, for example, no more than 12%, 11%, 10%, 9%, 8%, 7%, 6%, 5%, 4%, 3%, 2% or 1%. In some embodiments, the surfactant is present in an amount of 0.5% by weight.

Candidate surfactants (or other components) can be tested for suitability for use in the disclosure in a manner similar to that described for testing polymers.

One aspect of the disclosure provides a method of generating a spray dried dispersion comprising (i) providing a mixture of one or more APIs and a solvent; and (ii) forcing the mixture through a nozzle and subjecting the mixture to spray drying conditions to generate the spray dried dispersion.

Another aspect of the disclosure provides a method of generating a spray dried dispersion comprising: (i) providing a mixture comprising one or more APIs and a solvent(s); and (ii) forcing the mixture out of a nozzle under spray dry drying conditions to generate a spray dried dispersion.

Another aspect of the disclosure provides a method of generating a spray dried dispersion comprising (i) spraying a mixture through a nozzle, wherein the mixture comprises one or more APIs and a solvent; and (ii) forcing the mixture through a nozzle under spray drying conditions to generate a particle that comprises the APIs.

Another aspect of the disclosure provides a spray dried dispersion comprising one or more APIs, wherein the dispersion is substantially free of a polymer, and wherein the spray dried dispersion is generated by (i) providing a mixture that consists essentially of one or more APIs and a solvent; and (ii) forcing the mixture through a nozzle under spray drying conditions to generate the spray dried dispersion.

Another aspect of the disclosure provides a spray dried dispersion comprising one or more APIs, wherein the dispersion is generated by (i) providing a mixture that comprising one or more APIs, a polymer(s), and a solvent(s); and (ii) forcing the mixture through a nozzle under spray drying conditions to generate the spray dried dispersion.

Another aspect of the disclosure provides a spray dried dispersion comprising a particle, wherein the particle comprises one or more APIs and a polymer(s), and wherein the spray dried dispersion is generated by (i) spraying a mixture through a nozzle, wherein the mixture comprises one or more APIs and a solvent; and (ii) forcing the mixture through a nozzle under spray drying conditions to generate the spray dried dispersion.

Another aspect of the disclosure provides a spray dried dispersion comprising a particle, wherein the particle comprises one or more APIs, and the particle is substantially free of a polymer, and wherein the spray dried dispersion is generated by (i) spraying a mixture through a nozzle, wherein the mixture comprises one or more APIs and a solvent; and (ii) forcing the mixture through a nozzle under spray drying conditions to generate the spray dried dispersion.

In some embodiments, the one or more APIs are selected from Compound I, Compound II, and Compound III.

Some implementations further comprise further drying the spray dried dispersion. For example, the spray dried dispersion is dried under reduced pressure. In other examples, the spray dried dispersion is dried at a temperature of from 50° C. to 100° C.

In some implementations, the solvent comprises a polar organic solvent. Examples of polar organic solvents include methylethyl ketone, THF, DCM, methanol, or IPA, or any combination thereof, such as, for example DCM/methanol. In other examples, the solvent further comprises water. In other examples, the solvent further comprises water. For instance, the solvent could be methylethyl ketone/water, THF/water, or methylethyl ketone/water/IPA. For example, the ratio of the polar organic solvent to water is from 70:30 to 95:5 by volume. In other instances, the ratio of the polar organic solvent to water is 90:10 by volume.

Some implementations further comprise filtering the mixture before it is forced through the nozzle. Such filtering can be accomplished using any suitable filter media having a suitable pore size.

Some implementations further comprise applying heat to the mixture as it enters the nozzle. This heating can be accomplished using any suitable heating element.

In some implementations, the nozzle comprises an inlet and an outlet, and the inlet is heated to a temperature that is less than the boiling point of the solvent. For example, the inlet is heated to a temperature of from 90° C. to 150° C.

In some implementations, the mixture is forced through the nozzle by a pressurized gas. Examples of suitable pressurized gases include those pressurized gas that are inert to the first agent, the second agent, and the solvent. In one example, the pressurized gas comprises elemental nitrogen.

In some implementations, the pressurized gas has a positive pressure of from 90 psi to 150 psi.

Some implementations further comprise further drying the spray dried dispersion. For example, the spray dried dispersion is dried under reduced pressure. In other examples, the spray dried dispersion is dried at a temperature of from 50° C. to 100° C.

In some implementations, the solvent comprises a polar organic solvent. Examples of polar organic solvents include methylethyl ketone, THF, DCM, methanol, or IPA, or any combination thereof. In other examples, the solvent further comprises water. In other examples, the solvent further comprises water. For instance, the solvent could be methylethyl ketone/water, THF/water, or methylethyl ketone/water/IPA. For example, the ratio of the polar organic solvent to water is from 70:30 to 95:5 by volume. In other instances, the ratio of the polar organic solvent to water is 90:10 by volume.

In some implementations, a pharmaceutical composition of the disclosure comprising substantially amorphous API(s) (e.g., Compound I, Compound II, and/or Compound III) may be prepared by non-spray drying techniques, such as, for example, cyrogrounding/cryomilling techniques. A composition comprising substantially amorphous API(s) (e.g., Compound I, Compound II, and/or Compound III) may also be prepared by hot melt extrusion techniques.

In some embodiments, the solid dispersions (e.g., spray dried dispersions) of the disclosure comprise a polymer(s). Any suitable polymers known in the art can be used in the disclosure. Exemplary suitable polymers include polymers selected from cellulose-based polymers, polyoxyethylene-based polymers, polyethylene-propylene glycal copolymers, vinyl-based polymers, PEO-polyvinyl caprolactam-based polymers, and polymethacrylate-based polymers.

The cellulose-based polymers include a methylcellulose, a hydroxypropyl methylcellulose (HPMC) (hypromellose), a hypromellose phthalate (HPMC-P), a hypromellose acetate succinate, and co-polymers thereof. The polyoxyethylene-based polymers include a polyethylene-propylene glycol, a polyethylene glycol, a poloxamer, and co-polymers thereof. The vinyl-based polymers include a polyvinylpyrrolidine (PVP), and PVP/VA. The PEO-polyvinyl caprolactam-based polymers include a polyethylene glycol, polyvinyl acetate and polyvinylcaprolactam-based graft copolymer (e.g., Soluplus®). The polymethacrylate-based polymers are synthetic cationic and anionic polymers of dimethylaminoethyl methacrylates, methacrylic acid, and methacrylic acid esters in varying ratios. Several types are commercially available and may be obtained as the dr powder, aqueous dispersion, or organic solution. Examples of such polymethacrylate-based polymers include a poly(methacrylic acid, ethyl acrylate) (1:1), a dimethylaminoethyl methacrylate-methylmethacrylate copolymer, and a Eudragit®.

In some embodiments, the cellulose-based polymer is a hypromellose acetate succinate and a hypromellose, or a combination of hypromellose acetate succinate and a hypromellose.

In some embodiments, the cellulose-based polymer is hypromellose E15, hypromellose acetate succinate L or hypromellose acetate succinate H.

In some embodiments, the polyoxyethylene-based polymer or poly ethylene propylene glycol copolymer is a polyethylene glycol or a pluronic.

In some embodiments, the polyoxyethylene-based polymer or polyethylene-propylene glycol copolymer is polyethylene glycol 3350 or poloxamer 407.

In some embodiments, the vinyl-based polymer is a vinylpolyvinylpyrrolidine-based polymer, such as polyvinylpyrrolidine K30 or polyvinylpyrrolidine VA 64.

In some embodiments, the polymethacrylate polymer is Eudragit L100-55 or Eudragit® E PO.

In some embodiments, the polymer(s) is selected from cellulosic polymers such as HPMC and/or HPMCAS.

In some embodiments, the polymer(s) is selected from:

| | | | | Trade Name |
|---|---|---|---|---|
| Polymer class | Generic Name | Abbrev. | Grade | Example(s) |
| Cellulose based | Methyl cellulose | MC | | Methocel |
| Cellulose based | Hypromellose | HPMC | E15 | Methocel ™ |
| Cellulose based | Hypromellose | HPMCAS | L grade | AQOAT ®; |
| (pH-dependent) | acetate succinate | | | Affinisol ™ |
| Cellulose based | Hypromellose | HPMCAS | H grade | AQOAT ®; |
| (pH-dependent) | acetate succinate | | | Affinisol ™ |
| Polyoxyethylene- | Polyethylene | PEG | 3350 | Macrogol |
| based | glycol | | | |
| Polymethacrylate | Poly(methacrylic acid, | — | L100-55 | Eudragit ®; |
| | ethyl acrylate) (1:1) | | | ACRYL-EZE |
| Polymethacrylate | Dimethylaminoethyl | — | E PO | Eudragit ®; |
| | methacrylate- | | | ACRYL-EZE |
| | methylmethacrylate | | | |
| | copolymer | | | |
| Copolymer(random) | Polyvinylpyrrolidone/ | PVP-VA | 64 | Kollidon ® |
| | vinyl acetate (aka | | | VA64 |
| | copovidone) | | | |
| Vinyl-based polymer | Polyvinylpyrrolidone | PVP | K30 | Kollidon ® |
| Copolymer (graft) | Polyvinyl caprolactam- | — | Not | Soluplus ® |
| | polyvinyl acetate- | | applicable | |
| | polyethylene glycol | | | |
| | graft co-polymer | | | |
| copolymer (non- | Ethylene oxide/ | — | Poloxamer | Kolliphor ®; |
| ionic triblock) | propylene oxide | | 407 or | Pluronic ® |
| | block copolymer | | Pluronic ® | |
| | (aka Poloxamer) | | F127 | |

Exemplary polymers for Compound I SDD formulations are:

| | | | | Trade Name |
|---|---|---|---|---|
| Polymer class | Generic Name | Abbrev. | Grade | Example(s) |
| Cellulose based | Methyl cellulose | MC | | Methocel |
| Cellulose based | Hypromellose | HPMC | E15 | Methocel ™ |
| | (hydroxypropyl | | | |
| | methylcellulose) | | | |
| Cellulose based | Hypromellose acetate | HPMCAS | L grade | AQOAT ®; |
| (pH-dependent) | succinate | | | Affinisol ™ |
| Cellulose based | Hypromellose acetate | HPMCAS | H grade | AQOAT ®; |
| (pH-dependent) | succinate | | | Affinisol ™ |
| Polyoxyethylene- | Polyethylene glycol | PEG | 3350 | Macrogol |
| based | | | | |
| Polymethacrylate | Poly(methacrylic acid, | — | L100-55 | Eudragit ®; |
| | ethyl acrylate) (1:1) | | | ACRYL-EZE |
| Polymethacrylate | Dimethylaminoethyl | — | E PO | Eudragit ®; |
| | methacrylate- | | | ACRYL-EZE |
| | methylmethacrylate | | | |
| | copolymer | | | |
| Copolymer | Polyvinylpyrrolidone/ | PVP-VA | 64 | Kollidon ® |
| | vinyl acetate (aka | | | VA64 |
| | copovidone) | | | |
| Vinyl-based polymer | Polyvinylpyrrolidone | PVP | K30 | Kollidon ® |
| Copolymer (graft) | Polyvinyl caprolactam- | — | Not | Soluplus ® |
| | polyvinyl acetate- | | applicable | |
| | polyethylene glycol | | | |
| | graft co-polymer | | | |
| copolymer (non- | Ethylene oxide/ | — | Poloxamer | Kolliphor ®; |
| ionic triblock) | propylene oxide | | 407 or | Pluronic ® |
| | block copolymer | | Pluronic ® | |
| | (aka Poloxamer) | | F127 | |

In some embodiments, a polymer is able to dissolve in aqueous media. The solubility of the polymers may be pH independent or pH dependent. The latter include one or more enteric polymers. The term “enteric polymer” refers to a polymer that is preferentially soluble in the less acidic environment of the intestine relative to the more acid environment of the stomach, for example, a polymer that is insoluble in acidic aqueous media but soluble when the pH is above 5-6. An appropriate polymer should be chemically and biologically inert. In order to improve the physical stability of the solid dispersions, the glass transition temperature (Tg) of the polymer should be as high as possible. For example, polymers have a glass transition temperature at least equal to or greater than the glass transition temperature of the API. Other polymers have a glass transition temperature that is within 10 to 15° C. of the API.

Additionally, the hygroscopicity of the polymers should be as low, e.g., less than 10%. For the purpose of comparison in this application, the hygroscopicity of a polymer or composition is characterized at 60% relative humidity. In some embodiments, the polymer has less than 10% water absorption, for example less than 9%, less than 8%, less than 7%, less than 6%, less than 5%, less than 4%, less than 3%, or less than 2% water absorption. The hygroscopicity can also affect the physical stability of the solid dispersions. Generally, moisture adsorbed in the polymers can greatly reduce the Tg of the polymers as well as the resulting solid dispersions, which will further reduce the physical stability of the solid dispersions as described above.

In some embodiments, the polymer is one or more water-soluble polymer(s) or partially water-soluble polymer(s). Water-soluble or partially water-soluble polymers include but are not limited to, cellulose derivatives (e.g., hydroxypropylmethylcellulose (HPMC), hydroxypropylcellulose (HPC)) or ethylcellulose; polyvinylpyrrolidones (PVP); polyethylene glycols (PEG); polyvinyl alcohols (PVA); acrylates, such as polymethacrylate (e.g., Eudragit® E); cyclodextrins (e.g., β-cyclodextrin) and copolymers and derivatives thereof, including for example PVP-VA (polyvinylpyrrolidone-vinyl acetate).

In some embodiments, the polymer is hydroxypropylmethylcellulose (HPMC), such as HPMC E50, HPMC E15, or HPMC E3.

As discussed herein, the polymer can be a pH-dependent enteric polymer. Such pH-dependent enteric polymers include, but are not limited to, cellulose derivatives (e.g., cellulose acetate phthalate (CAP)), hydroxypropyl methyl cellulose phthalates (HPMCP), hydroxypropyl methyl cellulose acetate succinate (HPMCAS), carboxymethylcellulose (CMC) or a salt thereof (e.g., a sodium salt such as (CMC-Na)); cellulose acetate trimellitate (CAT), hydroxypropylcellulose acetate phthalate (HPCAP), hydroxypropylmethyl-cellulose acetate phthalate (HPMCAP), and methylcellulose acetate phthalate (MCAP), or polymethacrylates (e.g., Eudragit® S). In some embodiments, the polymer is hydroxypropyl methyl cellulose acetate succinate (HPMCAS). In some embodiments, the polymer is hydroxypropyl methyl cellulose acetate succinate HG grade (HPMCAS-HG).

In yet another embodiment, the polymer is a polyvinylpyrrolidone co-polymer, for example, a vinylpyrrolidone/vinyl acetate co-polymer (PVP/VA).

In embodiments where Compound I is in the form of a solid dispersion with a polymer, for example with an HPMC, HPMCAS, or PVP/VA polymer, the amount of polymer relative to the total weight of the solid dispersion ranges from 0.1% to 99% by weight. Unless otherwise specified, percentages of drug, polymer and other excipients as described within a dispersion are given in weight percentages. The amount of polymer is typically at least 20%, and such as at least 30%, for example, at least 35%, at least 40%, at least 45%, or 50% (e.g., 49.5%). The amount is typically 99% or less, and such as 80% or less, for example 75% or less, 70% or less, 65% or less, 60% or less, or 55% or less. In some embodiments, the polymer is present in an amount of up to 50% of the total weight of the dispersion (such as between 40% and 50%, such as 49%, 49.5%, or 50%).

In some embodiments, the API (e.g., Compound I, Compound II, or Compound III) and polymer are present in roughly equal amounts in weight, for example each of the polymer and the drug make up half of the percentage weight of the dispersion. For example, the polymer is present in 49.5 wt % and Compound I, Compound II, or Compound III is present in 50 wt %. In another embodiment, Compound I, Compound II, or Compound III is present in an amount greater than half of the percentage weight of the dispersion. For example, the polymer is present in an amount of 20 wt % and Compound I, Compound II, or Compound III is present in an amount of 80 wt %.

In some embodiments, the API (e.g., Compound I, Compound II, or Compound III) and the polymer combined represent 1% to 20% w/w total solid content of the spray drying solution prior to spray drying. In some embodiments, Compound I, Compound II, or Compound III, and the polymer combined represent 5% to 15% w/w total solid content of the spray drying solution prior to spray drying. In some embodiments, Compound I, Compound II, or Compound III, and the polymer combined represent 11% w/w total solid content of the spray drying solution prior to spray drying.

In some embodiments, the dispersion further includes other minor ingredients, such as a surfactant (e.g., SLS). In some embodiments, the surfactant is present in less than 10% of the dispersion, for example less than 9%, less than 8%, less than 7%, less than 6%, less than 5%, less than 4%, less than 3%, less than 2%, 1%, or 0.5%.

In embodiments including a polymer, the polymer should be present in an amount effective for stabilizing the solid dispersion. Stabilizing includes inhibiting or preventing, the crystallization of an API (e.g., Compound I, Compound II, or Compound III). Such stabilizing would inhibit the conversion of the API from amorphous to crystalline form. For example, the polymer would prevent at least a portion (e.g., 5%, 10%, 15%, 20%, 25%, 30%, 35%, 40%, 45%, 50%, 55%, 60%, 65%, 70%, 75%, or greater) of the API from converting from an amorphous to a crystalline form. Stabilization can be measured, for example, by measuring the glass transition temperature of the solid dispersion, measuring the amount of crystalline material, measuring the rate of relaxation of the amorphous material, or by measuring the solubility or bioavailability of the API.

In some embodiments, the polymers for use in the disclosure have a glass transition temperature of no less than 10-15° C. lower than the glass transition temperature of API. In some instances, the glass transition temperature of the polymer is greater than the glass transition temperature of API, and in general at least 50° C. higher than the desired storage temperature of the drug product. For example, at least 100° C., at least 105° C., at least 105° C., at least 110° C., at least 120° C., at least 130° C., at least 140° C., at least 150° C., at least 160° C., at least 160° C., or greater.

In some embodiments, the polymers for use in the disclosure have similar or better solubility in solvents suitable for spray drying processes relative to that of an API (e.g., Compound I, Compound II, or Compound III). In some embodiments, the polymer will dissolve in one or more of the same solvents or solvent systems as the API.

In some embodiments, the polymers for use in the disclosure can increase the solubility of an API (e.g., Compound I, Compound II, or Compound III) in aqueous and physiologically relative media either relative to the solubility of the API in the absence of polymer or relative to the solubility of the API when combined with a reference polymer. For example, the polymers can increase the solubility of Compound I, Compound II, or Compound III by reducing the amount of amorphous Compound I, Compound II, or Compound III that converts to a crystalline form(s), either from a solid amorphous dispersion or from a liquid suspension.

In some embodiments, the polymers for use in the disclosure can decrease the relaxation rate of the amorphous substance.

In some embodiments, the polymers for use in the disclosure can increase the physical and/or chemical stability of an API (e.g., Compound I, Compound II, or Compound III).

In some embodiments, the polymers for use in the disclosure can improve the manufacturability of an API (e.g., Compound I, Compound II, or Compound III).

In some embodiments, the polymers for use in the disclosure can improve one or more of the handling, administration or storage properties of an API (e.g., Compound I, Compound II, or Compound III).

In some embodiments, the polymers for use in the disclosure have little or no unfavorable interaction with other pharmaceutical components, for example excipients.

The suitability of a candidate polymer (or other component) can be tested using the spray drying methods (or other methods) described herein to form an amorphous composition. The candidate composition can be compared in terms of stability, resistance to the formation of crystals, or other properties, and compared to a reference preparation, e.g., a preparation of neat amorphous Compound I, Compound II, and Compound III. For example, a candidate composition could be tested to determine whether it inhibits the time to onset of solvent mediated crystallization, or the percent conversion at a given time under controlled conditions, by at least 50%, 75%, or 100% as well as the reference preparation, or a candidate composition could be tested to determine if it has improved bioavailability or solubility relative to crystalline Compound I, Compound II, or Compound III.

The spray dried dispersion of the present embodiment may include a surfactant as previously described.

B. Blends of Solid Dispersions

In some embodiments, the disclosure provides a pharmaceutical composition comprising a first solid dispersion comprising Compound I, a second solid dispersion comprising Compound II, and/or a third solid dispersion comprising Compound III.

In some embodiments, the first solid dispersion further comprises a cellulose polymer. For example, the first solid dispersion further comprises hydroxypropyl methylcellulose acetate succinate (HPMCAS).

In some embodiments, the second solid dispersion further comprises a cellulose polymer. For example, the second solid dispersion further comprises hydroxypropyl methylcellulose (HPMC). In some embodiments, the second solid dispersion comprises a weight ratio of HPMC to Compound II ranging from 1:10 to 1:1. In some instances, the ratio of HPMC to Compound II is from 1:3 to 1:5.

In some embodiments, the third solid dispersion further comprises a cellulose polymer. For example, the third solid dispersion further comprises hydroxypropyl methylcellulose acetate succinate (HPMCAS).

In some embodiments, each of the first, second and third solid dispersions comprises a plurality of particles having a mean particle diameter of 5 to 100 microns. In some embodiments, the particles have a mean particle diameter of 5 to 30 microns. In some embodiments, the particles have a mean particle diameter of 15 microns.

In some embodiments, the first solid dispersion comprises from 40 wt % to 90 wt % (e.g., from 75 wt % to 85 wt %) of Compound I.

In some embodiments, the first solid dispersion comprises from 70 wt % to 90 wt % (e.g., from 75 wt % to 85 wt %) of Compound I.

In some embodiments, the second solid dispersion comprises from 70 wt % to 90 wt % (e.g., from 75 wt % to 85 wt %) of Compound II.

In some embodiments, the third solid dispersion comprises from 70 wt % to 90 wt % (e.g., from 75 wt % to 85 wt %) of Compound III.

In some embodiments, each of the first, second, and third solid dispersions is a spray dried dispersion—the first, second, and third spray dried dispersions, respectively.

In some embodiments, the first spray dried dispersion further comprises a cellulose polymer. For example, the first spray dried dispersion further comprises hypromellose acetate succinate (HPMCAS).

In some embodiments, the second solid dispersion further comprises a cellulose polymer. For example, the second solid dispersion further comprises hydroxypropyl methylcellulose (HPMC).

In other embodiments, the third solid dispersion further comprises a cellulose polymer. For example, the solid dispersion further comprises hypromellose acetate succinate (HPMCAS).

One aspect of the disclosure provides a method of generating a pharmaceutical composition comprising (i) providing a first mixture comprising Compound I, a cellulose polymer, and a solvent; (ii) forcing the first mixture through a nozzle under spray drying conditions to generate the first spray dried dispersion comprising Compound I; (iii) providing a second mixture comprising Compound II, a cellulose polymer, and a solvent; (iv) forcing the second mixture through a nozzle under spray drying conditions to generate the second spray dried dispersion comprising Compound II; (v) providing a third mixture comprising Compound III, a cellulose polymer, a surfactant, and a solvent; (vi) forcing the third mixture through a nozzle under spray drying conditions to generate the third spray dried dispersion comprising Compound III; and (vii) combining the first spray dried dispersion, the second spray dried dispersion, and the third spray dried dispersion.

In some embodiments, the cellulose polymer of the second mixture is a HPMC.

In some embodiments, the second mixture comprises a ratio of HPMC to Compound II ranging from 3:7 to 1:9 by weight. For example, the ratio of HPMC to Compound I is from 3:7 to 1:5 (e.g., 1:4) by weight.

In some embodiments, the second mixture further comprises a solvent, and the solvent comprises a polar organic solvent. Examples of polar organic solvents include methylethyl ketone, THF, methanol, DCM, or IPA, or any combination thereof, such as for example, a DCM/methanol mixture. In other examples, the solvent further comprises water. In other examples, the solvent further comprises water. For instance, the solvent could be methylethyl ketone/water, THF/water, methanol/water, or methylethyl ketone/water/IPA. For example, the ratio of the polar organic solvent to water is from 70:30 to 95:5 by volume. In other instances, the ratio of the polar organic solvent to water is 90:10 by volume.

In some embodiments, the cellulose polymer of the first and third mixtures is independently a HPMCAS.

In some embodiments, the first mixture comprises a ratio of HPMCAS to Compound I ranging from 3:2 to 1:9 by weight. For example, the ratio of HPMCAS to Compound I is from 3:2 to 1:5 (e.g., 1:1 or 1:4) by weight.

In some embodiments, the third mixture further comprises a solvent, and the solvent comprises a polar organic solvent. Examples of polar organic solvents include methylethyl ketone, THF, methanol, DCM, or IPA, or any combination thereof, such as for example, a DCM/methanol mixture. In other examples, the solvent further comprises water. In other examples, the solvent further comprises water. For instance, the solvent could be methylethyl ketone/water, THF/water, methanol/water, or methylethyl ketone/water/IPA. For example, the ratio of the polar organic solvent to water is from 70:30 to 95:5 by volume. In other instances, the ratio of the polar organic solvent to water is 90:10 by volume.

Some embodiments further comprise filtering each of the first, second, and third mixtures before it is forced through the nozzle. Such filtering can be accomplished using any suitable filter media having a suitable pore size. Likewise, the second mixture may also be filtered before it is forced through the nozzle.

Some embodiments further comprise drying the first, second, and/or third spray dried dispersion. For example, the spray dried dispersion is dried under reduced pressure. In other examples, the spray dried dispersion is dried at a temperature of from 30° C. to 60° C.

C. Co-Spray Dried Dispersions Comprising Multiple APIs

Some embodiments of the disclosure provide a solid dispersion comprising one or more APIs (e.g., Compound I, Compound II and Compound III). In some embodiments, the solid dispersion is a spray dried dispersion prepared by co-spray drying a mixture of such APIs, a solvent, and a polymer. Suitable polymers are as described above.

In some embodiments, the solid dispersion comprises 50 mg to 800 mg of Compound I; 3 mg to 70 mg of Compound II; and 10 mg to 400 mg of Compound III. In some embodiments, the solid dispersion comprises Compounds I, II, and III in a weight ratio of Compound I:Compound II:Compound III 10 to 15:1:5 to 7. In some embodiments, the solid dispersion comprises Compounds I, II, and III in a weight ratio of Compound I:Compound II:Compound III 12:1:3 to 6. In some embodiments, the solid dispersion comprises Compounds I, II, and III in a weight ratio of Compound I:Compound II:Compound III 12:1:3. In some embodiments, the solid dispersion comprises Compounds I, II, and III in a weight ratio of Compound I:Compound II:Compound III 12:1:6.

In some embodiments, the solid dispersion further comprises a cellulosic polymer. For example, the solid dispersion comprises HPMC, HPMCAS, or any combination thereof.

In some embodiments of the disclosure provided is a pharmaceutical composition comprising a solid dispersion (e.g., a spray dried dispersion) and one or more excipients selected from a filler; a disintegrant; a surfactant; a binder; a wetting agent, a lubricant, or any combination thereof, wherein the solid dispersion comprises one or more APIs (e.g., Compound I, Compound II and Compound III).

In some embodiments, the solid dispersion is a spray dried dispersion, wherein the spray dried dispersion has a glass transition temperature (Tg) of from 80° C. to 180° C.

In some embodiments, the solid dispersion (e.g., a spray dried dispersion) comprises a plurality of particles having a mean particle diameter of 5 to 100 microns. In some embodiments, the solid dispersion (e.g., a spray dried dispersion) comprises a plurality of particles having a mean particle diameter of 5 to 30 microns. In some embodiments, the solid dispersion (e.g., a spray dried dispersion) comprises a plurality of particles having a mean particle diameter of 15 microns.

In some embodiments, the solid dispersion (e.g., a spray dried dispersion) is substantially amorphous.

Some embodiments of the disclosure provides a solid dispersion (e.g., a spray dried dispersion) comprising Compound I, Compound II and Compound III, wherein the solid dispersion is generated by (i) providing a mixture comprising Compound I, Compound II and Compound III and a solvent; and (ii) forcing the mixture through a nozzle under spray drying conditions to generate the solid dispersion.

In some embodiments, the solvent comprised in the mixture comprises a polar organic solvent. Examples of polar organic solvents include methylethyl ketone, THF, DCM, methanol, or IPA, or any combination thereof. In other examples, the solvent further comprises water. In other examples, the solvent further comprises water. For instance, the solvent could be methylethyl ketone/water, THF/water, or methylethyl ketone/water/IPA. For example, the ratio of the polar organic solvent to water is from 70:30 to 95:5 by volume. In other instances, the ratio of the polar organic solvent to water is 90:10 by volume.

Some embodiments further comprise filtering the mixture before it is forced through the nozzle. Such filtering can be accomplished using any suitable filter media having a suitable pore size.

Some embodiments further comprise drying the spray dried dispersion. For example, the spray dried dispersion is dried under reduced pressure. In other examples, the spray dried dispersion is dried at a temperature of from 30° C. to 60° C.

### PHARMACEUTICAL COMPOSITIONS

Some embodiments of the disclosure provide a pharmaceutical composition comprising any of the spray dried dispersions or combinations of spray dried dispersions described above and a pharmaceutically acceptable vehicle, adjuvant, or carrier.

A. Pharmaceutical compositions

Remington: The Science and Practice of Pharmacy, 21st edition, 2005, ed. D. B. Troy, Lippincott Williams & Wilkins, Philadelphia, and Encyclopedia of Pharmaceutical Technology, eds. J. Swarbrick and J. C. Boylan, 1988-1999, Marcel Dekker, New York, the contents of each of which is incorporated by reference herein, disclose various carriers used in formulating pharmaceutical compositions and known techniques for the preparation thereof. Except insofar as any conventional carrier medium is incompatible with the compounds of the disclosure, such as by producing any undesirable biological effect or otherwise interacting in a deleterious manner with any other component(s) of the pharmaceutical composition, its use is contemplated to be within the scope of this disclosure.

In some embodiments, the pharmaceutical compositions of the disclosure comprise a filler, a disintegrant, and a lubricant.

Fillers suitable for the disclosure are compatible with the ingredients of the pharmaceutical composition, i.e., they do not substantially reduce the solubility, the hardness, the chemical stability, the physical stability, or the biological activity of the pharmaceutical composition. Exemplary fillers include: celluloses, modified celluloses, (e.g. sodium carboxymethyl cellulose, ethyl cellulose hydroxymethyl cellulose, hydroxypropylcellulose), cellulose acetate, microcrystalline cellulose, calcium phosphates, dibasic calcium phosphate, starches (e.g. corn starch, potato starch), sugars (e.g., mannitol, lactose, sucrose, or the like), or any combination thereof. In some embodiments, the filler is microcrystalline cellulose.

In some embodiments, the pharmaceutical composition comprises at least one filler in an amount of at least 5 wt % (e.g., at least 20 wt %, at least 30 wt %, or at least 40 wt %) by weight of the composition. For example, the pharmaceutical composition comprises from 10 wt % to 60 wt % (e.g., from 20 wt % to 55 wt %, from 25 wt % to 50 wt %, or from 27 wt % to 45 wt %) of filler, by weight of the composition. In another example, the pharmaceutical composition comprises at least 20 wt % (e.g., at least 30 wt % or at least 40 wt %) of microcrystalline cellulose, for example MCC Avicel PH102 or Avicel PH101, by weight of the composition. In yet another example, the pharmaceutical composition comprises from 10 wt % to 60 wt % (e.g., from 20 wt % to 55 wt % or from 25 wt % to 45 wt %) of microcellulose, by weight of the composition.

Disintegrants suitable for the disclosure enhance the dispersal of the pharmaceutical composition and are compatible with the ingredients of the pharmaceutical composition, i.e., they do not substantially reduce the chemical stability, the physical stability, the hardness, or the biological activity of the pharmaceutical composition. Exemplary disintegrants include croscarmellose sodium, sodium starch glycolate, crospovidone or a combination thereof. In some embodiments, the disintegrant is croscarmellose sodium.

Thus, in some embodiments, the pharmaceutical composition comprises disintegrant in an amount of 10 wt % or less (e.g., 7 wt % or less, 6 wt % or less, or 5 wt % or less) by weight of the composition. For example, the pharmaceutical composition comprises from 1 wt % to 10 wt % (e.g., from 1.5 wt % to 7.5 wt % or from 2.5 wt % to 6 wt %) of disintegrant, by weight of the composition. In another example, the pharmaceutical composition comprises 10 wt % or less (e.g., 7 wt % or less, 6 wt % or less, or 5 wt % or less) of croscarmellose sodium, by weight of the composition. In yet another example, the pharmaceutical composition comprises from 1 wt % to 10 wt % (e.g., from 1.5 wt % to 7.5 wt % or from 2.5 wt % to 6 wt %) of croscarmellose sodium, by weight of the composition. In some examples, the pharmaceutical composition comprises from 0.1% to 10 wt % (e.g., from 0.5 wt % to 7.5 wt % or from 1.5 wt % to 6 wt %) of disintegrant, by weight of the composition. In still other examples, the pharmaceutical composition comprises from 0.5% to 10 wt % (e.g., from 1.5 wt % to 7.5 wt % or from 2.5 wt % to 6 wt %) of disintegrant, by weight of the composition.

In some embodiments, the pharmaceutical composition can include an oral solid pharmaceutical dosage form which can comprise a lubricant that can prevent adhesion of a granulate-bead admixture to a surface (e.g., a surface of a mixing bowl, a compression die and/or punch). A lubricant can also reduce interparticle friction within the granulate and improve the compression and ejection of compressed pharmaceutical compositions from a die press. The lubricant is also compatible with the ingredients of the pharmaceutical composition, i.e., they do not substantially reduce the solubility, the hardness, or the biological activity of the pharmaceutical composition. Exemplary lubricants include magnesium stearate, sodium stearyl fumarate, calcium stearate, zinc stearate, sodium stearate, stearic acid, aluminum stearate, leucine, glyceryl behenate, hydrogenated vegetable oil or any combination thereof. In embodiment, the lubricant is magnesium stearate.

In some embodiments, the pharmaceutical composition comprises a lubricant in an amount of 5 wt % or less (e.g., 4.75 wt %, 4.0 wt % or less, or 3.00 wt % or less, or 2.0 wt % or less) by weight of the composition. For example, the pharmaceutical composition comprises from 5 wt % to 0.10 wt % (e.g., from 4.5 wt % to 0.5 wt % or from 3 wt % to 1 wt %) of lubricant, by weight of the composition. In another example, the pharmaceutical composition comprises 5 wt % or less (e.g., 4.0 wt % or less, 3.0 wt % or less, or 2.0 wt % or less, or 1.0 wt % or less) of magnesium stearate, by weight of the composition. In yet another example, the pharmaceutical composition comprises from 5 wt % to 0.10 wt % (e.g., from 4.5 wt % to 0.15 wt % or from 3.0 wt % to 0.50 wt %) of magnesium stearate, by weight of the composition.

In some embodiments, the pharmaceutical composition includes or can be made into tablets and the tablets can be coated with a film coating and optionally labeled with a logo, other image and/or text using a suitable ink. In still other embodiments, the pharmaceutical composition includes or can be made into tablets and the tablets can be coated with a film coating, waxed, and optionally labeled with a logo, other image and/or text using a suitable ink. Suitable film coatings and inks are compatible with the ingredients of the pharmaceutical composition, i.e., they do not substantially reduce the solubility, the chemical stability, the physical stability, the hardness, or the biological activity of the pharmaceutical composition. The suitable colorants and inks can be any color and are water based or solvent based. In some embodiments, tablets made from the pharmaceutical composition are coated with a colorant and then labeled with a logo, other image, and/or text using a suitable ink. For example, tablets comprising pharmaceutical composition as described herein can be coated with 3 wt % (e.g., less than 6 wt % or less than 4 wt %) of film coating comprising one or more colorants/pigments. The colored tablets can be labeled with a logo and text indicating the strength of the active ingredient in the tablet using a suitable ink. In another example, tablets comprising pharmaceutical composition as described herein can be coated with 3 wt % (e.g., less than 6 wt % or less than 4 wt %) of a film coating comprising one or more colorants/pigments.

The tablets of the disclosure can be produced by compacting or compressing an admixture or composition, for example, powder or granules, under pressure to form a stable three-dimensional shape (e.g., a tablet). As used herein, “tablet” includes compressed pharmaceutical dosage unit forms of all shapes and sizes, whether coated or uncoated.

Granulation and Compression

In some embodiments, solid forms, including powders comprising one or more APIs (e.g., Compound I, Compound II, and/or Compound III) and the included pharmaceutically acceptable excipients (e.g. filler, diluent, disintegrant, surfactant, glidant, binder, lubricant, or any combination thereof) can be subjected to a dry granulation process. The dry granulation process causes the powder to agglomerate into larger particles having a size suitable for further processing. Dry granulation can improve the flowability of a mixture to produce tablets that comply with the demand of mass variation or content uniformity.

In some embodiments, formulations can be produced using one or more mixing and dry granulations steps. The order and the number of the mixing by granulation. At least one of the excipients and the API(s) can be subject to dry granulation or wet high shear granulation or twin screw wet granulation before compression into tablets. Dry granulation can be carried out by a mechanical process, which transfers energy to the mixture without any use of any liquid substances (neither in the form of aqueous solutions, solutions based on organic solutes, or mixtures thereof) in contrast to wet granulation processes, also contemplated herein. Generally, the mechanical process requires compaction such as the one provided by roller compaction. An example of an alternative method for dry granulation is slugging. In some embodiments, wet granulations instead of the dry granulation can be used.

In some embodiments, roller compaction is a granulation process comprising mechanical compacting of one or more substances. In some embodiments, a pharmaceutical composition comprising an admixture of powders is pressed, that is roller compacted, between two rotating rollers to make a solid sheet that is subsequently crushed in a sieve to form a particulate matter. In this particulate matter, a close mechanical contact between the ingredients can be obtained. An example of roller compaction equipment is Minipactor® a Gerteis 3W-Polygran from Gerteis Maschinen+Processengineering AG.

In some embodiments, tablet compression according to the disclosure can occur without any use of any liquid substances (neither in the form of aqueous solutions, solutions based on organic solutes, or mixtures thereof), i.e., a dry granulation process. In a typical embodiment the resulting core or tablet has a compressive strength in the range of from 1 kp to 15 kP; such as 1.5 to 12.5 kP, such as in the range of 2 to 10 kP.

Brief Manufacturing Procedure

In some embodiments, the ingredients are weighed according to the formula set herein. Next, all of the intragranular ingredients are sifted and mixed well. The ingredients can be lubricated with a suitable lubricant, for example, magnesium stearate. The next step can comprise compaction/slugging of the powder admixture and sized ingredients. Next, the compacted or slugged blends are milled into granules and sifted to obtain the desired size. Next, the granules can be further lubricated with, for example, magnesium stearate. Next, the granular composition of the disclosure can be compressed on suitable punches into various pharmaceutical formulations in accordance with the disclosure. Optionally the tablets can be coated with a film coat.

Some embodiments of the disclosure provide a method for producing a pharmaceutical composition comprising an admixture of a composition comprising one or more APIs (e.g., Compound I, Compound II and/or Compound III); and one or more excipients selected from: a filler, a diluent, a binder, a glidant, a surfactant, a lubricant, a disintegrant, and compressing the composition into a tablet having a dissolution of at least 50% in 30 minutes.

Suitable pharmaceutically acceptable salts are, for example, those disclosed in S. M. Berge, et al. J. Pharmaceutical Sciences, 1977, 66, 1-19. For example, Table 1 of that article provides the following pharmaceutically acceptable salts:

TABLE 1

| | | |
|---|---|---|
| Acetate | Iodide | Benzathine |
| Benzenesulfonate | Isethionate | Chloroprocaine |
| Benzoate | Lactate | Choline |
| Bicarbonate | Lactobionate | Diethanolamine |
| Bitartrate | Malate | Ethylenediamine |
| Bromide | Maleate | Meglumine |
| Calcium edetate | Mandelate | Procaine |
| Camsylate | Mesylate | Aluminum |
| Carbonate | Methylbromide | Calcium |
| Chloride | Methylnitrate | Lithium |
| Citrate | Methylsulfate | Magnesium |
| Dihydrochloride | Mucate | Potassium |
| Edetate | Napsylate | Sodium |
| Edisylate | Nitrate | Zinc |
| Estolate | Pamoate (Embonate) | |
| Esylate | Pantothenate | |
| Fumarate | Phosphate/diphosphate | |
| Gluceptate | Polygalacturonate | |
| Gluconate | Salicylate | |
| Glutamate | Stearate | |
| Glycollylarsanilate | Subacetate | |
| Hexylresorcinate | Succinate | |
| Hydrabamine | Sulfate | |
| Hydrobromide | Tannate | |
| Hydrochloride | Tartrate | |
| Hydroxynaphthoate | Teociate | |
| | Triethiodide | |

Non-limiting examples of pharmaceutically acceptable acid addition salts include: salts formed with inorganic acids, such as hydrochloric acid, hydrobromic acid, phosphoric acid, sulfuric acid, or perchloric acid; salts formed with organic acids, such as acetic acid, oxalic acid, maleic acid, tartaric acid, citric acid, succinic acid or malonic acid; and salts formed by using other methods used in the art, such as ion exchange. Non-limiting examples of pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, and valerate salts. Pharmaceutically acceptable salts derived from appropriate bases include alkali metal, alkaline earth metal, ammonium, and N⁺(C₁₋₄alkyl)₄ salts. This disclosure also envisions the quaternization of any basic nitrogen-containing groups of the compounds disclosed herein. Suitable non-limiting examples of alkali and alkaline earth metal salts include sodium, lithium, potassium, calcium, and magnesium. Further non-limiting examples of pharmaceutically acceptable salts include ammonium, quaternary ammonium, and amine cations formed using counterions such as halide, hydroxide, carboxylate, sulfate, phosphate, nitrate, lower alkyl sulfonate and aryl sulfonate. Other suitable, non-limiting examples of pharmaceutically acceptable salts include besylate and glucosamine salts.

In some embodiments, at least one compound chosen from Compound I and pharmaceutically acceptable salts thereof is administered in combination with Compound II or a pharmaceutically acceptable salt thereof. In some embodiments, at least one compound chosen from Compound I and pharmaceutically acceptable salts thereof is administered in combination with Compound III or a pharmaceutically acceptable salt thereof. In some embodiments, at least one compound chosen from Compound I and pharmaceutically acceptable salts thereof is administered in combination with Compounds II or a pharmaceutically acceptable salt thereof and Compound III or a pharmaceutically acceptable salt thereof. In some embodiments, at least one compound chosen from Compound I and pharmaceutically acceptable salts thereof is administered in combination with Compound III.

Each of Compounds I, II, and III, and their pharmaceutically acceptable salts thereof independently can be administered once daily, twice daily, or three times daily. In some embodiments, Compound I or its pharmaceutically acceptable salts thereof are administered once daily. In some embodiments, Compound I or its pharmaceutically acceptable salts thereof are administered twice daily. In some embodiments, Compound II or its pharmaceutically acceptable salts thereof are administered once daily. In some embodiments, Compound II or its pharmaceutically acceptable salts thereof are administered twice daily. In some embodiments, Compound III or its pharmaceutically acceptable salts thereof are administered once daily. In some embodiments, Compound III or its pharmaceutically acceptable salts thereof are administered twice daily.

In some embodiments, Compound I or its pharmaceutically acceptable salts are administered in an amount ranging from 600 mg to 1600 mg, 1000 mg to 1400 mg, 1000 mg to 1200 mg, 1200 mg to 1600 mg, 1200 mg to 1400 mg, or 1400 mg to 1600 mg, daily. In some embodiments, Compound I or its pharmaceutically acceptable salts are administered in an amount of 600 mg, 700 mg, 800 mg, 900 mg, 1000 mg, 1100, 1200 mg, 1300 mg, 1400 mg, 1500 mg, or 1600 mg, daily. In some embodiments, Compound I or its pharmaceutically acceptable salts are administered in an amount of 600 mg, 700 mg, 800 mg, 900 mg, 1000 mg, 1100, 1200 mg, 1300 mg, 1400 mg, 1500 mg, or 1600 mg once daily. In some embodiments, Compound I or its pharmaceutically acceptable salts are administered in an amount of 300 mg, 400 mg, 500 mg, 600 mg, 700 mg, or 800 mg twice daily.

In some embodiments, Compound II or its pharmaceutically acceptable salts are administered in an amount ranging from 25 mg to 200 mg, 50 mg to 150 mg, 50 mg to 200 mg, or 75 mg to 200 mg, daily. In some embodiments, Compound II or its pharmaceutically acceptable salts are administered in an amount of 50 mg or 100 mg daily. In some embodiments, Compound II or its pharmaceutically acceptable salts are administered in an amount of 50 mg or 100 mg once daily. In some embodiments, Compound II or its pharmaceutically acceptable salts are administered in an amount of 50 mg or 100 mg twice daily. In some embodiments, Compound II or its pharmaceutically acceptable salts are administered in an amount of 100 mg once daily.

In some embodiments, Compound III or its pharmaceutically acceptable salts are administered in an amount ranging from 50 mg to 800 mg, 50 mg to 700 mg, 100 mg to 400 mg, 150 mg to 700 mg, 200 mg to 700 mg, or 500 mg to 700 mg, daily. In some embodiments, Compound III or its pharmaceutically acceptable salts are administered in an amount of 50 mg, 75 mg, 100 mg, 150 mg, 200 mg, 300 mg, or 600 mg, daily. In some embodiments, Compound III or its pharmaceutically acceptable salts are administered in an amount of 50 mg, 75 mg, 100 mg, 150 mg, 200 mg, 300 mg, or 600 mg, once daily. In some embodiments, Compound III or its pharmaceutically acceptable salts are administered in an amount of 50 mg, 75 mg, 100 mg, 150 mg, 200 mg, or 300 mg, twice daily. In some embodiments, Compound III or its pharmaceutically acceptable salts are administered in an amount of 150 mg or 300 mg twice daily.

Compounds I, II, and III, and their pharmaceutically acceptable salts of any of the foregoing can be comprised in a single pharmaceutical composition or separate pharmaceutical compositions. Such pharmaceutical compositions can be administered once daily or multiple times daily, such as twice daily.

In some embodiments, at least one compound chosen from Compound I and pharmaceutically acceptable salts thereof is comprised in a first pharmaceutical composition; at least one compound chosen from Compound II and pharmaceutically acceptable salts thereof is comprised in a second pharmaceutical composition; and at least one compound chosen from Compound III and pharmaceutically acceptable salts thereof is comprised in a third pharmaceutical composition.

In some embodiments, at least one compound chosen from Compound I and pharmaceutically acceptable salts thereof is comprised in a first pharmaceutical composition; and at least one compound chosen from Compound II and pharmaceutically acceptable salts thereof and at least one compound chosen from Compound III and pharmaceutically acceptable salts thereof are comprised in a second pharmaceutical composition. In some embodiments, the second pharmaceutical composition comprises a half of a daily dose of said at least one compound chosen from Compound III and pharmaceutically acceptable salts thereof, and the other half of the daily dose of said at least one compound chosen from Compound III and pharmaceutically acceptable salts thereof is administered in a third pharmaceutical composition.

In some embodiments, at least one compound chosen from Compound I and pharmaceutically acceptable salts thereof is comprised in a first pharmaceutical composition; at least one compound chosen from Compound II and pharmaceutically acceptable salts thereof is comprised in a second pharmaceutical composition; and at least one compound chosen from Compound III and pharmaceutically acceptable salts thereof is comprised in a first pharmaceutical composition. In some embodiments, the first pharmaceutical composition is administered to the patient twice daily.

In some embodiments, the disclosure features a pharmaceutical composition comprising Compound I or a pharmaceutically acceptable salt thereof, and at least one pharmaceutically acceptable carrier.

In some embodiments, the disclosure features a pharmaceutical composition comprising Compound I or a pharmaceutically acceptable salt thereof, Compound II or a pharmaceutically acceptable salt thereof, and at least one pharmaceutically acceptable carrier.

In some embodiments, the disclosure features a pharmaceutical composition comprising Compound I or a pharmaceutically acceptable salt thereof, Compound III or a pharmaceutically acceptable salt thereof, and at least one pharmaceutically acceptable carrier.

In some embodiments, the disclosure features a pharmaceutical composition comprising Compound I or a pharmaceutically acceptable salt thereof, Compound II or a pharmaceutically acceptable salt thereof, Compound III or a pharmaceutically acceptable salt thereof, and at least one pharmaceutically acceptable carrier.

In some embodiments, the disclosure features a pharmaceutical composition comprising Compound I or a pharmaceutically acceptable salt thereof, Compound III or a pharmaceutically acceptable salt thereof, and at least one pharmaceutically acceptable carrier.

In some embodiments, pharmaceutical compositions disclosed herein comprise at least one additional API (active pharmaceutical ingredient). In some embodiments, the at least one additional active pharmaceutical ingredient is a CFTR modulator. In some embodiments, the at least one additional active pharmaceutical ingredient is a CFTR corrector. In some embodiments, the at least one additional active pharmaceutical ingredient is a CFTR potentiator. In some embodiments, the pharmaceutical composition comprises Compound I and at least two additional active pharmaceutical ingredients, one of which is a CFTR corrector and one of which is a CFTR potentiator.

In some embodiments, at least one additional active pharmaceutical ingredient is selected from mucolytic agents, bronchodilators, antibiotics, anti-infective agents, and anti-inflammatory agents.

A pharmaceutical composition may further comprise at least one pharmaceutically acceptable carrier. In some embodiments, the at least one pharmaceutically acceptable carrier is chosen from pharmaceutically acceptable vehicles and pharmaceutically acceptable adjuvants. In some embodiments, the at least one pharmaceutically acceptable is chosen from pharmaceutically acceptable fillers, disintegrants, surfactants, binders, lubricants.

It will also be appreciated that a pharmaceutical composition of this disclosure, including a pharmaceutical composition comprising combinations described previously, can be employed in combination therapies; that is, the compositions can be administered concurrently with, prior to, or subsequent to, at least one additional active pharmaceutical ingredient or medical procedures.

Pharmaceutical compositions comprising these combinations are useful for treating cystic fibrosis.

In some embodiments, a pharmaceutical composition disclosed herein comprises at least one compound chosen from Compound I and pharmaceutically acceptable salts thereof, and at least one pharmaceutically acceptable carrier. In some embodiments, the pharmaceutically acceptable carrier is a polymer. In some embodiments, the pharmaceutically acceptable carrier is HPMCAS. In some embodiments, the pharmaceutically acceptable carrier is HPMCAS-HG. In some embodiments, the pharmaceutical composition comprises a solid dispersion of compound I in HPMCAS-HG. In some embodiments, the solid dispersion comprises compound I to HPMCAS-HG in a 1:1 weight ratio. In some embodiments, the solid dispersion comprises compound I to HPMCAS-HG in a 4:1 weight ratio. In some embodiments, the solid dispersion comprises substantially amorphous compound I.

As described above, pharmaceutical compositions disclosed herein may optionally further comprise at least one pharmaceutically acceptable carrier. The at least one pharmaceutically acceptable carrier may be chosen from adjuvants and vehicles. The at least one pharmaceutically acceptable carrier, as used herein, includes any and all solvents, diluents, other liquid vehicles, dispersion aids, suspension aids, surface active agents, isotonic agents, thickening agents, emulsifying agents, preservatives, solid binders, and lubricants, as suited to the particular dosage form desired. Remington: The Science and Practice of Pharmacy, 21st edition, 2005, ed. D. B. Troy, Lippincott Williams & Wilkins, Philadelphia, and Encyclopedia of Pharmaceutical Technology, eds. J. Swarbrick and J. C. Boylan, 1988-1999, Marcel Dekker, New York discloses various carriers used in formulating pharmaceutical compositions and known techniques for the preparation thereof. Except insofar as any conventional carrier is incompatible with the compounds of this disclosure, such as by producing any undesirable biological effect or otherwise interacting in a deleterious manner with any other component(s) of the pharmaceutical composition, its use is contemplated to be within the scope of this disclosure. Non-limiting examples of suitable pharmaceutically acceptable carriers include, but are not limited to, ion exchangers, alumina, aluminum stearate, lecithin, serum proteins (such as human serum albumin), buffer substances (such as phosphates, glycine, sorbic acid, and potassium sorbate), partial glyceride mixtures of saturated vegetable fatty acids, water, salts, and electrolytes (such as protamine sulfate, disodium hydrogen phosphate, potassium hydrogen phosphate, sodium chloride, and zinc salts), colloidal silica, magnesium trisilicate, polyvinyl pyrrolidone, polyacrylates, waxes, polyethylene-polyoxypropylene-block polymers, wool fat, sugars (such as lactose, glucose and sucrose), starches (such as corn starch and potato starch), cellulose and its derivatives (such as sodium carboxymethyl cellulose, ethyl cellulose and cellulose acetate), powdered tragacanth, malt, gelatin, talc, excipients (such as cocoa butter and suppository waxes), oils (such as peanut oil, cottonseed oil, safflower oil, sesame oil, olive oil, corn oil and soybean oil), glycols (such as propylene glycol and polyethylene glycol), esters (such as ethyl oleate and ethyl laurate), agar, buffering agents (such as magnesium hydroxide and aluminum hydroxide), alginic acid, pyrogen-free water, isotonic saline, Ringer's solution, ethyl alcohol, phosphate buffer solutions, non-toxic compatible lubricants (such as sodium lauryl sulfate and magnesium stearate), coloring agents, releasing agents, coating agents, sweetening agents, flavoring agents, perfuming agents, preservatives, and antioxidants.

It will also be appreciated that a pharmaceutical composition of this disclosure, including a pharmaceutical composition comprising any of the combinations described previously, can be employed in combination therapies; that is, the compositions can be administered concurrently with, prior to, or subsequent to, at least one active pharmaceutical ingredients or medical procedures.

In some embodiments, the methods of the disclosure employ administering to a patient in need thereof Compound I or a pharmaceutically acceptable salt thereof; and at least one selected from Compound II, Compound III, and pharmaceutically acceptable salts thereof.

Any suitable pharmaceutical compositions known in the art can be used for Compound I, Compound II, Compound III, and pharmaceutically acceptable salts of any of the foregoing. Some exemplary pharmaceutical compositions for Compound I and its pharmaceutically acceptable salts are described in the Examples. Some exemplary pharmaceutical compositions for Compound II and its pharmaceutically acceptable salts can be found in WO 2011/119984 and WO 2014/015841, all of which are incorporated herein by reference. Some exemplary pharmaceutical compositions for Compound III and its pharmaceutically acceptable salts can be found in WO 2007/134279, WO 2010/019239, WO 2011/019413, WO 2012/027731, and WO 2013/130669, all of which are incorporated herein by reference.

In some embodiments, a pharmaceutical composition comprising at least one Compound I and pharmaceutically acceptable salts thereof is administered with a pharmaceutical composition comprising Compound II and Compound III. Pharmaceutical compositions comprising Compound II and Compound III are disclosed in PCT Publication No. WO 2015/160787, incorporated herein by reference. An exemplary embodiment is shown in the following Table:

TABLE 2
Exemplary Tablet Comprising 100 mg of
Compound II and 150 mg of Compound III.

| | | Amount per |
|---|---|---|
| | Ingredient | tablet (mg) |
| Intra-granular | Compound II SDD (spray | 125 |
| | dried dispersion) | |
| | (80 wt % Compound II; | |
| | 20 wt % HPMC) | |
| | Compound III SDD (80 wt % | 187.5 |
| | Compound III; 19.5 wt % | |
| | HPMCAS-HG; 0.5 wt % | |
| | sodium lauryl sulfate) | |
| | Microcrystalline cellulose | 131.4 |
| | Croscarmellose Sodium | 29.6 |
| | Total | 473.5 |
| Extra-granular | Microcrystalline cellulose | 112.5 |
| | Magnesium Stearate | 5.9 |
| | Total | 118.4 |
| Total uncoated Tablet | | 591.9 |
| Film coat | Opadry | 17.7 |
| Total coated Tablet | | 609.6 |

In some embodiments, a pharmaceutical composition comprising Compound I is administered with a pharmaceutical composition comprising Compound III. Pharmaceutical compositions comprising Compound III are disclosed in PCT Publication No. WO 2010/019239, incorporated herein by reference. An exemplary embodiment is shown in the following Table:

TABLE 3
Ingredients for Exemplary Tablet of Compound III.

| | Percent | | |
|---|---|---|---|
| | Dose | Dose | Batch |
| Tablet Formulation | % Wt./Wt. | (mg) | (g) |
| Compound III SDD | 34.09% | 187.5 | 23.86 |
| (80 wt % Compound III; | | | |
| 19.5 wt % HPMCAS-HG; | | | |
| 0.5 wt % sodium lauryl | | | |
| sulfate) | | | |
| Microcrystalline cellulose | 30.51% | 167.8 | 21.36 |
| Lactose | 30.40% | 167.2 | 21.28 |
| Sodium croscarmellose | 3.000% | 16.50 | 2.100 |
| SLS | 0.500% | 2.750 | 0.3500 |
| Colloidal silicon dioxide | 0.500% | 2.750 | 0.3500 |
| Magnesium stearate | 1.000% | 5.500 | 0.7000 |
| Total | 100% | 550 | 70 |

Additional pharmaceutical compositions comprising Compound III are disclosed in PCT Publication No. WO 2013/130669, incorporated herein by reference. Exemplary mini-tablets (˜2 mm diameter, ˜2 mm thickness, each mini-tablet weighing about 6.9 mg) was formulated to have approximately 50 mg of Compound III per 26 mini-tablets and approximately 75 mg of Compound III per 39 mini-tablets using the amounts of ingredients recited in Table 4, below.

TABLE 4
Ingredients for mini-tablets for 50 mg and 75 mg potency

| | Percent | Dose (mg) | Dose (mg) | |
|---|---|---|---|---|
| | Dose | 50 mg | 75 mg | Batch |
| Tablet Formulation | % Wt./Wt. | potency | potency | (g) |
| Compound III SDD (80 | 35 | 62.5 | 93.8 | 1753.4 |
| wt % Compound III; | | | | |
| 19.5 wt % HPMCAS-HG; | | | | |
| 0.5 wt % sodium lauryl | | | | |
| sulfate) | | | | |
| Mannitol | 13.5 | 24.1 | 36.2 | 675.2 |
| Lactose | 41 | 73.2 | 109.8 | 2050.2 |
| Sucralose | 2.0 | 3.6 | 5.4 | 100.06 |
| Croscarmellose sodium | 6.0 | 10.7 | 16.1 | 300.1 |
| Colloidal silicon dioxide | 1.0 | 1.8 | 2.7 | 50.0 |
| Magnesium stearate | 1.5 | 2.7 | 4.0 | 74.19 |
| Total | 100 | 178.6 | 268 | 5003.15 |

In some embodiments, disclosed herein are pharmaceutical compositions comprising:

| | Amount per tablet (wt % |
|---|---|
| | relative to the total |
| Ingredient | weight of the tablet) |
| Compound I | 25-36 |
| Compound II | 2-4 |
| Compound III | 13-18 |
| Croscarmellose Sodium | 5-7 |
| Microcrystalline cellulose | 16-23 |
| Magnesium Stearate | 0.5-2 |

In some embodiments, disclosed herein are pharmaceutical compositions comprising:

| | Amount per tablet (wt % relative |
|---|---|
| Ingredient | to the total weight of the tablet) |
| Compound I | 33-36 |
| Compound II | 2-3 |
| Compound III | 17-18 |
| Croscarmellose Sodium | 5-7 |
| Microcrystalline cellulose | 22-23 |
| Magnesium Stearate | 0.5-1.5 |

In some embodiments, disclosed herein are pharmaceutical compositions comprising:

| | Amount per tablet (wt % relative |
|---|---|
| Ingredient | to the total weight of the tablet) |
| Compound I | 25-28 |
| Compound II | 2-3 |
| Compound III | 12-14 |
| Croscarmellose Sodium | 5-7 |
| Microcrystalline cellulose | 16-18 |
| Magnesium Stearate | 0.5-1.5 |

In some embodiments, in any of the pharmaceutical compositions disclosed herein, Compounds I, II, and III are in a weight ratio of Compound I:Compound II:Compound III 10 to 15:1:5 to 7.

In some embodiments, in any of the pharmaceutical compositions disclosed herein, Compounds I, II, and III are in a weight ratio of Compound I:Compound II:Compound III 12:1:3 to 6.

In some embodiments, in any of the pharmaceutical compositions disclosed herein, Compounds I, II, and III are in a weight ratio of Compound I:Compound II:Compound III 12:1:6.

In some embodiments, in any of the pharmaceutical compositions disclosed herein, Compound I, Compound II, and Compound III are each independently in a solid dispersion comprising 70 wt % to 90 wt % of the respective compound: a solid dispersion comprising 70 wt % to 90 wt % of Compound I, a solid dispersion comprising 70 wt % to 90 wt % of Compound II, and a solid dispersion comprising 70 wt % to 90 wt % of Compound III.

In some embodiments, in any of the pharmaceutical compositions disclosed herein, Compound I, Compound II, and Compound III are each independently in a solid dispersion comprising 80 wt % of the respective compound: a solid dispersion comprising 80 wt % of Compound I, a solid dispersion comprising 80 wt % of Compound II, and a solid dispersion comprising 80 wt % of Compound III.

In some embodiments, in any of the pharmaceutical compositions disclosed herein, Compound I, Compound II, and Compound III are each independently in a solid dispersion comprising 70 wt % to 90 wt % of the respective compound: a solid dispersion comprising 70 wt % to 90 wt % of Compound I, a solid dispersion comprising 70 wt % to 90 wt % of Compound II, and a solid dispersion comprising 70 wt % to 90 wt % of Compound III, and wherein Compound I, Compound II, and Compound III are in a weight ratio of 12:1:6 Compound 1: Compound II: Compound III.

In some embodiments, in any of the pharmaceutical compositions disclosed herein, Compound I, Compound II, and Compound III are each independently in a solid dispersion comprising 80 wt % of the respective compound: a solid dispersion comprising 80 wt % of Compound I, a solid dispersion comprising 80 wt % of Compound II, and a solid dispersion comprising 80 wt % of Compound III, and wherein Compound I, Compound II, and Compound III are in a weight ratio of 12:1:6 Compound 1: Compound II: Compound III.

In some embodiments, the pharmaceutical compositions disclosed herein are tablets. In some embodiments, the tablets are suitable for oral administration.

These combinations are useful for treating cystic fibrosis.

A CFTR mutation may affect the CFTR quantity, i.e., the number of CFTR channels at the cell surface, or it may impact CFTR function, i.e., the functional ability of each channel to open and transport ions. Mutations affecting CFTR quantity include mutations that cause defective synthesis (Class I defect), mutations that cause defective processing and trafficking (Class II defect), mutations that cause reduced synthesis of CFTR (Class V defect), and mutations that reduce the surface stability of CFTR (Class VI defect). Mutations that affect CFTR function include mutations that cause defective gating (Class III defect) and mutations that cause defective conductance (Class IV defect).

In some embodiments, disclosed herein methods of treating, lessening the severity of, or symptomatically treating cystic fibrosis in a patient comprising administering an effective amount of a compound, pharmaceutically acceptable salt thereof, or a deuterated analog of any of the foregoing; or a pharmaceutical composition, of this disclosure to a patient, such as a human, wherein said patient has cystic fibrosis. In some embodiments, the patient has F508del/minimal function (MF) genotypes, F508del/F508del genotypes, F508del/gating genotypes, or F508del/residual function (RF) genotypes.

As used herein, “minimal function (MF) mutations” refer to CFTR gene mutations associated with minimal CFTR function (little-to-no functioning CFTR protein) and include, for example, mutations associated with severe defects in ability of the CFTR channel to open and close, known as defective channel gating or “gating mutations”; mutations associated with severe defects in the cellular processing of CFTR and its delivery to the cell surface; mutations associated with no (or minimal) CFTR synthesis; and mutations associated with severe defects in channel conductance. Table C below includes a non-exclusive list of CFTR minimal function mutations, which are detectable by an FDA-cleared genotyping assay. In some embodiments, a mutation is considered a MF mutation if it meets at least 1 of the following 2 criteria:
(1) biological plausibility of no translated protein (genetic sequence predicts the complete absence of CFTR protein), or
(2) in vitro testing that supports lack of responsiveness to Compound II, Compound III or the combination of Compound II and Compound III, and evidence of clinical severity on a population basis (as reported in large patient registries).

In some embodiments, the minimal function mutations are those that result in little-to-no functioning CFTR protein and are not responsive in vitro to Compound II, Compound III, or the combination of Compound II and Compound III.

In some embodiments, the minimal function mutations are those that are not responsive in vitro to Compound II, Compound III, or the combination of Compound II and Compound III. In some embodiments, the minimal function mutations are mutations based on in vitro testing met the following criteria in in vitro experiments:
baseline chloride transport that was <10% of wildtype CFTR, and
an increase in chloride transport of <10% over baseline following the addition of TEZ, IVA, or TEZ/IVA in the assay.
   In some embodiments, patients with at least one minimal function mutation exhibit evidence of clinical severity as defined as:
average sweat chloride>86 mmol/L, and
prevalence of pancreatic insufficiency (PI)>50%.

Patients with an F508del/minimal function genotype are defined as patients that are heterozygous F508del-CFTR with a second CFTR allele containing a a minimal function mutation. In some embodiments, patients with an F508del/minimal function genotype are patients that are heterozygous F508del-CFTR with a second CFTR allele containing a mutation that results in a CFTR protein with minimal CFTR function (little-to-no functioning CFTR protein) and that is responsive in vitro to Compound II, Compound III, or the combination of Compound II and Compound III.

In some embodiments, minimal function mutations can be using 3 major sources:
biological plausibility for the mutation to respond (i.e., mutation class)
evidence of clinical severity on a population basis (per CFTR2 patient registry;
accessed on 15 Feb. 2016)
   average sweat chloride>86 mmol/L, and
   prevalence of pancreatic insufficiency (PI)>50%
in vitro testing
   mutations resulting in baseline chloride transport<10% of wild-type CFTR were considered minimal function
   mutations resulting in chloride transport<10% of wild-type CFTR following the addition of Compound II and/or Compound III were considered nonresponsive.

As used herein, a “residual function mutations” refer to are Class II through V mutations that have some residual chloride transport and result in a less severe clinical phenotype. Residual function mutations are mutation in the CFTR gene that result in reduced protein quantity or function at the cell surface which can produce partial CFTR activity.

Non-limiting examples of CFTR gene mutations known to result in a residual function phenotype include a CFTR residual function mutation selected from 2789+5G→A, 3849+1 OkbC→T, 3272-26A→G, 711+3A→G, E56K, P67L, R74W, DllOE, Dl lOH, R117C, L206W, R347H, R352Q, A455E, D579G, E831X, S945L, S977F, F1052V, R1070W, F1074L, Dl 152H, D1270N, E193K, and K1060T. For example, CFTR mutations that cause defective mRNA splicing, such as 2789+507 A, result in reduced protein synthesis, but deliver some functional CFTR to the surface of the cell to provide residual function. Other CFTR mutations that reduce conductance and/or gating, such as RI 17H, result in a normal quantity of CFTR channels at the surface of the cell, but the functional level is low, resulting in residual function. In some embodiments, the CFTR residual function mutation is selected from R117H, S1235R, I1027T, R668C, G576A, M470V, L997F, R75Q, R1070Q, R31C, D614G, G1069R, R1162L, E56K, A1067T, E193K, and K1060T. In some embodiments, the CFTR residual function mutation is selected from R117H, S1235R, I1027T, R668C, G576A, M470V, L997F, R75Q, R1070Q, R31C, D614G, G1069R, R1162L, E56K, and A1067T.

Residual CFTR function can be characterized at the cellular (in vitro) level using cell based assays, such as an FRT assay (Van Goar, F. et al. (2009) PNAS Vol. 106, No. 44, 18825-18830; and Van Goor, F. et al. (2011) PNAS Vol. 108, No. 46, 18843-18846), to measure the amount of chloride transport through the mutated CFTR channels. Residual function mutations result in a reduction but not complete elimination of CFTR dependent ion transport. In some embodiments, residual function mutations result in at least about 10% reduction of CFTR activity in an FRT assay. In some embodiments, the residual function mutations result in up to about 90% reduction in CFTR activity in an FRT assay.

Patients with an F508del/residual function genotype are defined as patients that are heterozygous F508del-CFTR with a second CFTR allele that contains a mutation that results in reduced protein quantity or function at the cell surface which can produce partial CFTR activity.

Patients with an F508del/gating mutation genotype are defined as patients that are heterozygous F508del-CFTR with a second CFTR allele that contains a mutation associated with a gating defect and clinically demonstrated to be responsive to Compound III. Examples of such mutations include: G178R, S549N, S549R, G551D, G551S, G1244E, S1251N, S1255P, and G1349D.

In some embodiments, the methods of treating, lessening the severity of, or symptomatically treating cystic fibrosis disclosed herein are each independently produces an increase in chloride transport above the baseline chloride transport of the patient.

In some embodiments, in the methods of treating, lessening the severity of, or symptomatically treating cystic fibrosis disclosed herein, the patient is heterozygous for F508del, and the other CFTR genetic mutation is any CF-causing mutation. In some embodiments, the patient is heterozygous for F508del, and the other CFTR genetic mutation is any CF-causing mutation, and is expected to be and/or is responsive to any of the novel compounds disclosed herein, such as Compound 1, Compound II, Compound III and/or Compound IV genotypes based on in vitro and/or clinical data. In some embodiments, the patient is heterozygous for F508del, and the other CFTR genetic mutation is any CF-causing mutation, and is expected to be and/or is responsive to any combinations of (i) the novel compounds disclosed herein, such as Compound 1, and (ii) Compound II, and/or Compound III and/or Compound IV genotypes based on in vitro and/or clinical data.

In some embodiments, in the methods of treating, lessening the severity of, or symptomatically treating cystic fibrosis disclosed herein, the patient possesses a CFTR genetic mutation selected from any of the mutations listed in Table A.

TABLE A
CF Mutations

| 078delT | 1949del84 | | 3121−2A→G |
|---|---|---|---|
| 1078delT | 2043delG | | 3121-977_3499+248del2515 |
| 11234V | 2055del9→A | | 3132delTG |
| 1154insTC | 2105-2117del13insAGAAA | | 3141del9 |
| 1161delC | 2118del14 | | 3171delC |
| 1213delT | 2143delT | | 3195del6 |
| 1248+1G→A | 2183AA−>G+ | | 3199del6 |
| 1249−1G→A | 2183AA→G | | 3272−26A−>G |
| 124del23bp | 2183AA→G^{a} | | 3500−2A→G |
| 1259insA | 2183delAA−>G# | | 3600+2insT |
| 1288insTA | 2183delAA→G | | 365-366insT |
| 1341+1G−>A | 2184delA | | 3659delC |
| 1342−2A−>C | 2184insA | | 3667ins4 |
| 1461ins4 | 2307insA | | 3737delA |
| 1471delA | 2347delG | | 3791delC |
| 1497delGG | 2556insAT | | 3821delT |
| 1507del | 2585delT | | 3849+10kbC→T |
| 1525−1G→A | 2594delGT | | 3849+lOkbC−>T |
| 1525−2A→G | 2622+1G−>A | | 3850−1G→A |
| 1548delG | 2622+IG→A | | 3850−3T−>G |
| 1577delTA | 2659delC | | 3850−IG−>A |
| 1609del CA | 2711delT | | 3876delA |
| 1677delTA | 271delT | | 3878delG |
| 1716G/A | 2721del11 | | 3905InsT |
| 1717−1G→A | 2732insA | | 3905insT |
| 1717−8G→A | 2789+2insA | | 394delTT |
| 1782delA | 2789+5G→A | | 4005+1G−>A |
| 1811+1.6kbA−>G | 2790−1G→C | | 4005+2T−>C |
| 1811+1G−>C | 2790−IG−>C | | 4005+1G→A |
| 1811+1.6kbA→G | 2869insG | | 4005+IG−>A |
| 1811+1G→C | 2896insAG | | 4010del4 |
| 1812−1G−>A | 2942insT | | 4015delA |
| 1898+1G−>A | 2957delT | | 4016insT |
| 1812−1G→A | 296+1G→A | | 4021dupT |
| 1824delA | 2991del32 | | 4040delA |
| 182delT | 3007delG | | 405+1G→A |
| 1119delA | 3028delA | | 405+3A→C |
| 185+1G→T | 3040G→C | | 405+IG−>A |
| 1898+1G−>T | 306insA | | 406−1G→A |
| 1898+1G→A | 306insA | 1138insG | 406−IG−>A |

| | | |
|---|---|---|
| 1898+1G→C | 3120G→A | 4209TGTT−>A |
| 1898+3A−>G | 3121−1G→A | 4209TGTT→AA |
| 1898+5G−>T | D1152H | 4279insA |
| 1924del7 | D1270N | G194R |
| 4326delTC | D192G | G194V |
| 4374+1G→T | D443Y | G27R |
| 4374+IG−>T | D513G | G27X |
| 4382delA | D579G | G314E |
| 4428insGA | D614G | G330X |
| 442delA | D836Y | G458V |
| 457TAT→G | D924N | G463V |
| 541delC | D979V | G480C |
| 574delA | E1104X | G542X |
| 5T | E116K | G550X |
| 621+1G→T | E1371X | G551D |
| 621+3A−>G | E193K | G551S |
| 663delT | E193X | G576A |
| 663delT | E403D | G622D |
| 1548delG | E474K | G628R |
| 675del4 | E56K | G628R(G−>A) |
| 711+1G−>T | E585X | G970D |
| 711+3A−>G | E588V | G673X |
| 711+1G→T | E60K | G85E |
| 711+3A→G | E822K | G91R |
| 711+5G→A | E822X | G970R |
| 712−1G−>T | E831X | G970R |
| 7T | E92K | H1054D |
| 852del22 | E92X | H1085P |
| 935delA | F1016S | H1085R |
| 991del5 | F1052V | H1375P |
| A1006E | F1074L | H139R |
| A120T | F1099L | H199R |
| A234D | F191V | H199Y |
| A349V | F311del | H609R |
| A455E | F311L | H939R |
| A613T | F508C | I1005R |
| A46D | F508del | I1027T |
| A46Db | F575Y | I1234V |
| A559T | G1061R | I1269N |
| A559Tb | G1069R | I1366N |
| A561E | G1244E | I148T |
| C276X | G1249R | I175V |
| C524R | G126D | I3336K |
| C524X | G1349D | I502T |
| CFTRdel2,3 | G149R | I506S |
| CFTRdele22-23 | G178R | I506T |
| D110E | P205S | I507del |
| D110H | P574H | R117P |
| I507del | P5L | R1283M |
| I601F | P67L | R1283S |
| I618T | P750L | R170H |
| I807M | P99L | R258G |
| I980K | Q1100P | R31C |
| IVS14b+5G−>A | Q1291H | R31L |
| K710X | Q1291R | R334L |
| K710X | Q1313X | R334Q |
| K710X | Q1382X | R334W |
| L102R | Q1411X | R347H |
| L1065P | Q1412X | R347L |
| L1077P | Q220X | R347P |
| L1077Pb | Q237E | R352Q |
| L1254X | Q237H | R352W |
| L1324P | Q452P | R516G |
| L1335P | Q290X | R553Q |
| L138ins | Q359K/T360K | R553X |
| L1480P | Q39X | R560K |
| L15P | Q414 | R560S |
| L165S | Q414X | R560T |
| L206W | E585X | R668C |
| L218X | Q493X | R709X |
| L227R | Q525X | R74W |
| L320V | Q552X | R751L |
| L346P | Q685X | R75Q |
| L453S | Q890X | R75X |
| L467P | Q890X | R764X |
| L467Pb | Q98R | R792G |
| L558S | Q98X | R792X |
| L571S | R1066C | R851X |
| L732X | R1066H | R933G |
| L927P | R1066M | S1118F |
| L967S | R1070Q | S1159F |
| L997F | R1070W | S1159P |
| M1101K | R1102X | S1196X |
| M1101R | R1158X | S1235R |
| M152V | R1162L | S1251N |
| M1T | R1162X | S1255X |
| M1V | R117C | S13F |
| M265R | R117G | S341P |
| M470V | R117H | S434X |
| M952I | R117L | S466X |
| M952T | Y1092X | |
| N1303K | Y109N | |
| S489X | Y122X | |
| S492F | Y161D | |
| S4X | Y161S | |
| S549N | Y563D | |
| S549R | Y563N | |
| S549R(A−>C) | Y569C | |
| S549R(T−>G) | Y569D | |
| S589N | Y569Db | |
| S737F | Y849X | |
| S912L | Y913C | |
| S912X | Y913X | |
| S945L | | |
| S977F | | |
| T1036N | | |
| T1053I | | |
| T1246I | | |
| T338I | | |
| T604I | | |
| V1153E | | |
| V1240G | | |
| V1293G | | |
| V201M | | |
| V232D | | |
| V456A | | |
| V456F | | |
| V520F | | |
| V562I | | |
| V754M | | |
| W1089X | | |
| W1098C | | |
| W1098R | | |
| W1098X | | |
| W1204X | | |
| W1282R | | |
| W1282X | | |
| W361R | | |
| W401X | | |
| W496X | | |
| W57G | | |
| W57R | | |
| W57X | | |
| W846X | | |
| Y1014C | | |
| Y1032C | | |

In some embodiments, in the methods of treating, lessening the severity of, or symptomatically treating cystic fibrosis disclosed herein, the patient possesses a CFTR genetic mutation selected from G178R, G551S, G970R, G1244E, S1255P, G1349D, S549N, S549R, S1251N, E193K, F1052V, G1069R, R117C, D110H, R347H, R352Q, E56K, P67L, L206W, A455E, D579G, S1235R, S945L, R1070W, F1074L, D110E, D1270N, D1152H, 1717-1G->A, 621+1G->T, 3120+1G->A, 1898+1G->A, 711+1G->T, 2622+1G->A, 405+1G->A, 406-1G->A, 4005+1G->A, 1812-1G->A, 1525-1G->A, 712-1G->T, 1248+1G->A, 1341+1G->A, 3121-1G->A, 4374+1G->T, 3850-1G->A, 2789+5G->A, 3849+10kbC->T, 3272-26A->G, 711+5G->A, 3120G->A, 1811+1.6kbA->G, 711+3A->G, 1898+3A->G, 1717-8G->A, 1342-2A->C, 405+3A->C, 1716G/A, 1811+1G->C, 1898+5G->T, 3850-3T->G, IVS14b+5G->A, 1898+1G->T, 4005+2T->C, 621+3A->G, 1949del84, 3141del9, 3195del6, 3199del6, 3905InsT, 4209TGTT->A, A1006E, A120T, A234D, A349V, A613T, C524R, D192G, D443Y, D513G, D836Y, D924N, D979V, E116K, E403D, E474K, E588V, E60K, E822K, F1016S, F1099L, F191V, F311del, F311L, F508C, F575Y, G1061R, G1249R, G126D, G149R, G194R, G194V, G27R, G314E, G458V, G463V, G480C, G622D, G628R, G628R(G->A), G91R, G970D, H1054D, H1085P, H1085R, H1375P, H139R, H199R, H609R, H939R, 11005R, I1234V, I1269N, I1366N, I175V, 1502T, 1506S, 1506T, I601F, I618T, 1807M, 1980K, L102R, L1324P, L1335P, L138ins, L1480P, LISP, L165S, L320V, L346P, L453S, L571S, L967S, M1101R, M152V, M1T, M1V, M265R, M9521, M952T, P574H, PSL, P750L, P99L, Q1100P, Q1291H, Q1291R, Q237E, Q237H, Q452P, Q98R, R1066C, R1066H, R117G, R117L, R117P, R1283M, R1283S, R170H, R258G, R31L, R334L, R334Q, R347L, R352W, R516G, R553Q, R751L, R792G, R933G, S1118F, S1159F, S1159P, S13F, S549R(A->C), S549R(T->G), S589N, S737F, S912L, T1036N, T10531, T12461, T6041, V1153E, V1240G, V1293G, V201M, V232D, V456A, V456F, V5621, W1098C, W1098R, W1282R, W361R, W57G, W57R, Y1014C, Y1032C, Y109N, Y161D, Y161S, Y563D, Y563N, Y569C, and Y913C.

In some embodiments, the patient has at least one combination mutation chosen from: G178R, G551S, G970R, G1244E, S1255P, G1349D, S549N, S549R, S1251N, E193K, F1052V, G1069R, R117C, D110H, R347H, R352Q, E56K, P67L, L206W, A455E, D579G, S1235R, S945L, R1070W, F1074L, D110E, D1270N, D1152H, 1717-1G->A, 621+1G->T, 3120+1G->A, 1898+1G->A, 711+1G->T, 2622+1G->A, 405+1G->A, 406-1G->A, 4005+1G->A, 1812-1G->A, 1525-1G->A, 712-1G->T, 1248+1G->A, 1341+1G->A, 3121-1G->A, 4374+1G->T, 3850-1G->A, 2789+5G->A, 3849+10kbC->T, 3272-26A->G, 711+5G->A, 3120G->A, 1811+1.6kbA->G, 711+3A->G, 1898+3A->G, 1717-8G->A, 1342-2A->C, 405+3A->C, 1716G/A, 1811+1G->C, 1898+5G->T, 3850-3T->G, IVS14b+5G->A, 1898+1G->T, 4005+2T->C, and 621+3A->G.

In some embodiments, the patient has at least one combination mutation chosen from: 1949del84, 3141del9, 3195del6, 3199del6, 3905InsT, 4209TGTT->A, A1006E, A120T, A234D, A349V, A613T, C524R, D192G, D443Y, D513G, D836Y, D924N, D979V, E116K, E403D, E474K, E588V, E60K, E822K, F1016S, F1099L, F191V, F311del, F311L, F508C, F575Y, G1061R, G1249R, G126D, G149R, G194R, G194V, G27R, G314E, G458V, G463V, G480C, G622D, G628R, G628R(G->A), G91R, G970D, H1054D, H1085P, H1085R, H1375P, H139R, H199R, H609R, H939R, 11005R, I1234V, I1269N, I1366N, I175V, 1502T, 1506S, 1506T, I601F, I618T, 1807M, 1980K, L102R, L1324P, L1335P, L138ins, L1480P, LISP, L165S, L320V, L346P, L453S, L571S, L967S, M1101R, M152V, M1T, M1V, M265R, M9521, M952T, P574H, PSL, P750L, P99L, Q1100P, Q1291H, Q1291R, Q237E, Q237H, Q452P, Q98R, R1066C, R1066H, R117G, R117L, R117P, R1283M, R1283S, R170H, R258G, R31L, R334L, R334Q, R347L, R352W, R516G, R553Q, R751L, R792G, R933G, S1118F, S1159F, S1159P, S13F, S549R(A->C), S549R(T->G), S589N, S737F, S912L, T1036N, T10531, T12461, T6041, V1153E, V1240G, V1293G, V201M, V232D, V456A, V456F, V5621, W1098C, W1098R, W1282R, W361R, W57G, W57R, Y1014C, Y1032C, Y109N, Y161D, Y161S, Y563D, Y563N, Y569C, and Y913C.

In some embodiments, in the methods of treating, lessening the severity of, or symptomatically treating cystic fibrosis disclosed herein, the patient possesses a CFTR genetic mutation G551D. In some embodiments, the patient is homozygous for the G551D genetic mutation. In some embodiments, the patient is heterozygous for the G551D genetic mutation. In some embodiments, the patient is heterozygous for the G551D genetic mutation, having the G551D mutation on one allele and any other CF-causing mutation on the other allele. In some embodiments, the patient is heterozygous for the G551D genetic mutation on one allele and the other CF-causing genetic mutation on the other allele is any one of F508del, G542X, N1303K, W1282X, R117H, R553X, 1717-1G->A, 621+1G->T, 2789+5G->A, 3849+10kbC->T, R1162X, G85E, 3120+1G->A, ΔI1507, 1898+1G->A, 3659delC, R347P, R560T, R334W, A455E, 2184delA, or 711+1G->T. In some embodiments, the patient is heterozygous for the G551D genetic mutation, and the other CFTR genetic mutation is F508del. In some embodiments, the patient is heterozygous for the G551D genetic mutation, and the other CFTR genetic mutation is R117H.

In some embodiments, in the methods of treating, lessening the severity of, or symptomatically treating cystic fibrosis disclosed herein, the patient possesses a CFTR genetic mutation F508del. In some embodiments, the patient is homozygous for the F508del genetic mutation. In some embodiments, the patient is heterozygous for the F508del genetic mutation wherein the patient has the F508del genetic mutation on one allele and any CF-causing genetic mutation on the other allele. In some embodiments, the patient is heterozygous for F508del, and the other CFTR genetic mutation is any CF-causing mutation, including, but not limited to G551D, G542X, N1303K, W1282X, R117H, R553X, 1717-1G->A, 621+1G->T, 2789+5G->A, 3849+10kbC->T, R1162X, G85E, 3120+1G->A, ΔI1507, 1898+1G->A, 3659delC, R347P, R560T, R334W, A455E, 2184delA, or 711+1G->T. In some embodiments, the patient is heterozygous for F508del, and the other CFTR genetic mutation is G551D. In some embodiments, the patient is heterozygous for F508del, and the other CFTR genetic mutation is R117H.

In some embodiments, the patient has at least one combination mutation chosen from:

### D443Y;G576A;R668C,

### F508C;S1251N,

### G576A; R668C,

### G970R; M470V,

### R74W;D1270N,

### R74W;V201M, and

### R74W;V201M;D1270N.

In some embodiments, in the methods of treating, lessening the severity of, or symptomatically treating cystic fibrosis disclosed herein, the patient possesses a CFTR genetic mutation selected from G178R, G551S, G970R, G1244E, S1255P, G1349D, S549N, S549R, S1251N, E193K, F1052V and G1069R. In some embodiments, the patient possesses a CFTR genetic mutation selected from G178R, G551S, G970R, G1244E, S1255P, G1349D, S549N, S549R and S1251N. In some embodiments, the patient possesses a CFTR genetic mutation selected from E193K, F1052V and G1069R. In some embodiments, the method produces an increase in chloride transport relative to baseline chloride transport of the patient of the patient.

In some embodiments, in the methods of treating, lessening the severity of, or symptomatically treating cystic fibrosis disclosed herein, the patient possesses a CFTR genetic mutation selected from R117C, D110H, R347H, R352Q, E56K, P67L, L206W, A455E, D579G, S1235R, S945L, R1070W, F1074L, D110E, D1270N and D1152H.

In some embodiments, the patient possesses a CFTR genetic mutation selected from 1717-1G->A, 621+1G->T, 3120+1G->A, 1898+1G->A, 711+1G->T, 2622+1G->A, 405+1G->A, 406-1G->A, 4005+1G->A, 1812-1G->A, 1525-1G->A, 712-1G->T, 1248+1G->A, 1341+1G->A, 3121-1G->A, 4374+1G->T, 3850-1G->A, 2789+5G->A, 3849+10kbC->T, 3272-26A->G, 711+5G->A, 3120G->A, 1811+1.6kbA->G, 711+3A->G, 1898+3A->G, 1717-8G->A, 1342-2A->C, 405+3A->C, 1716G/A, 1811+1G->C, 1898+5G->T, 3850-3T->G, IVS14b+5G->A, 1898+1G->T, 4005+2T->C and 621+3A->G. In some embodiments, the patient possesses a CFTR genetic mutation selected from 1717-1G->A, 1811+1.6kbA->G, 2789+5G->A, 3272-26A->G and 3849+10kbC->T. In some embodiments, the patient possesses a CFTR genetic mutation selected from 2789+5G->A and 3272-26A->G.

In some embodiments, in the methods of treating, lessening the severity of, or symptomatically treating cystic fibrosis disclosed herein, the patient possesses a CFTR genetic mutation selected from G178R, G551S, G970R, G1244E, S1255P, G1349D, S549N, S549R, S1251N, E193K, F1052V, G1069R, R117C, D110H, R347H, R352Q, E56K, P67L, L206W, A455E, D579G, S1235R, S945L, R1070W, F1074L, D110E, D1270N, D1152H, 1717-1G->A, 621+1G->T, 3120+1G->A, 1898+1G->A, 711+1G->T, 2622+1G->A, 405+1G->A, 406-1G->A, 4005+1G->A, 1812-1G->A, 1525-1G->A, 712-1G->T, 1248+1G->A, 1341+1G->A, 3121-1G->A, 4374+1G->T, 3850-1G->A, 2789+5G->A, 3849+10kbC->T, 3272-26A->G, 711+5G->A, 3120G->A, 1811+1.6kbA->G, 711+3A->G, 1898+3A->G, 1717-8G->A, 1342-2A->C, 405+3A->C, 1716G/A, 1811+1G->C, 1898+5G->T, 3850-3T->G, IVS14b+5G->A, 1898+1G->T, 4005+2T->C and 621+3A->G, and human CFTR mutations selected from F508del, R117H, and G551D.

In some embodiments, in the methods of treating, lessening the severity of, or symptomatically treating cystic fibrosis disclosed herein, the patient possesses a CFTR genetic mutation selected from G178R, G551S, G970R, G1244E, S1255P, G1349D, S549N, S549R, S1251N, E193K, F1052V, G1069R, R117C, D110H, R347H, R352Q, E56K, P67L, L206W, A455E, D579G, S1235R, S945L, R1070W, F1074L, D110E, D1270N, D1152H, 1717-1G->A, 621+1G->T, 3120+1G->A, 1898+1G->A, 711+1G->T, 2622+1G->A, 405+1G->A, 406-1G->A, 4005+1G->A, 1812-1G->A, 1525-1G->A, 712-1G->T, 1248+1G->A, 1341+1G->A, 3121-1G->A, 4374+1G->T, 3850-1G->A, 2789+5G->A, 3849+10kbC->T, 3272-26A->G, 711+5G->A, 3120G->A, 1811+1.6kbA->G, 711+3A->G, 1898+3A->G, 1717-8G->A, 1342-2A->C, 405+3A->C, 1716G/A, 1811+1G->C, 1898+5G->T, 3850-3T->G, IVS14b+5G->A, 1898+1G->T, 4005+2T->C, 621+3A->G, and a CFTR mutation selected from F508del, R117H, and G551D; and a CFTR mutations selected from F508del, R117H, and G551D.

In some embodiments, the patient possesses a CFTR genetic mutation selected from G178R, G551S, G970R, G1244E, S1255P, G1349D, S549N, S549R, S1251N, E193K, F1052V and G1069R, and a human CFTR mutation selected from F508del, R117H, and G551D. In some embodiments, the patient possesses a CFTR genetic mutation selected from G178R, G551S, G970R, G1244E, S1255P, G1349D, S549N, S549R and S1251N, and a human CFTR mutation selected from F508del, R117H, and G551D. In some embodiments, the patient possesses a CFTR genetic mutation selected from E193K, F1052V and G1069R, and a human CFTR mutation selected from F508del, R117H, and G551D.

In some embodiments, the patient possesses a CFTR genetic mutation selected from R117C, D110H, R347H, R352Q, E56K, P67L, L206W, A455E, D579G, S1235R, S945L, R1070W, F1074L, D110E, D1270N and D1152H, and a human CFTR mutation selected from F508del, R117H, and G551D.

In some embodiments, the patient possesses a CFTR genetic mutation selected from 1717-1G->A, 621+1G->T, 3120+1G->A, 1898+1G->A, 711+1G->T, 2622+1G->A, 405+1G->A, 406-1G->A, 4005+1G->A, 1812-1G->A, 1525-1G->A, 712-1G->T, 1248+1G->A, 1341+1G->A, 3121-1G->A, 4374+1G->T, 3850-1G->A, 2789+5G->A, 3849+10kbC->T, 3272-26A->G, 711+5G->A, 3120G->A, 1811+1.6kbA->G, 711+3A->G, 1898+3A->G, 1717-8G->A, 1342-2A->C, 405+3A->C, 1716G/A, 1811+1G->C, 1898+5G->T, 3850-3T->G, IVS14b+5G->A, 1898+1G->T, 4005+2T->C and 621+3A->G, and a human CFTR mutation selected from F508del, R117H, and G551D. In some embodiments, the patient possesses a CFTR genetic mutation selected from 1717-1G->A, 1811+1.6kbA->G, 2789+5G->A, 3272-26A->G and 3849+10kbC->T, and a human CFTR mutation selected from F508del, R117H, and G551D. In some embodiments, the patient possesses a CFTR genetic mutation selected from 2789+5G->A and 3272-26A->G, and a human CFTR mutation selected from F508del, R117H.

In some embodiments, the patient is heterozygous having a CF-causing mutation on one allele and a CF-causing mutation on the other allele. In some embodiments, the patient is heterozygous for F508del, and the other CFTR genetic mutation is any CF-causing mutation, including, but not limited to F508del on one CFTR allele and a CFTR mutation on the second CFTR allele that is associated with minimal CFTR function, residual CFTR function, or a defect in CFTR channel gating activity.

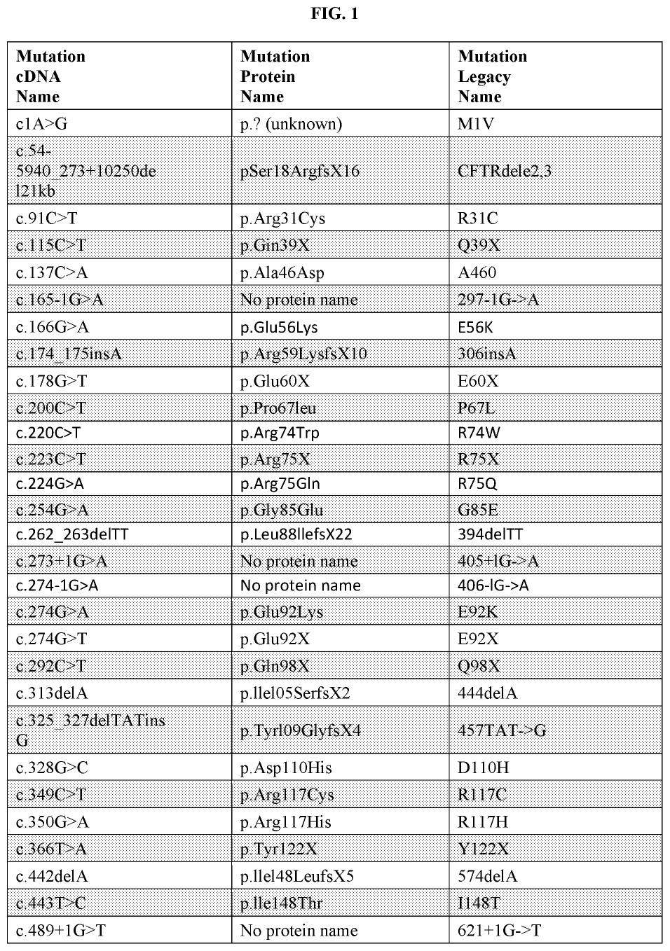

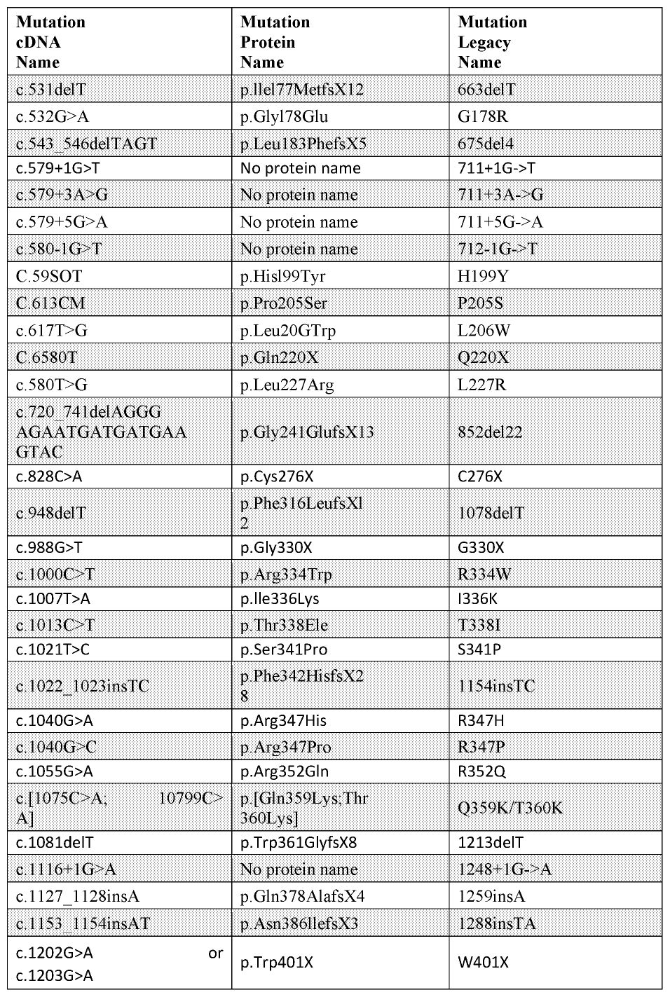

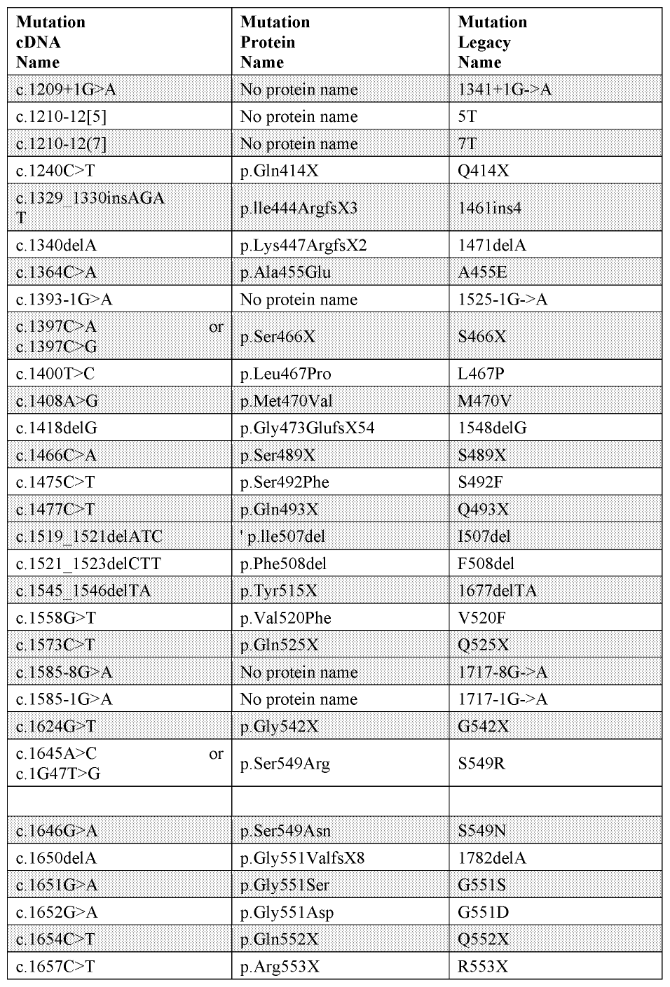

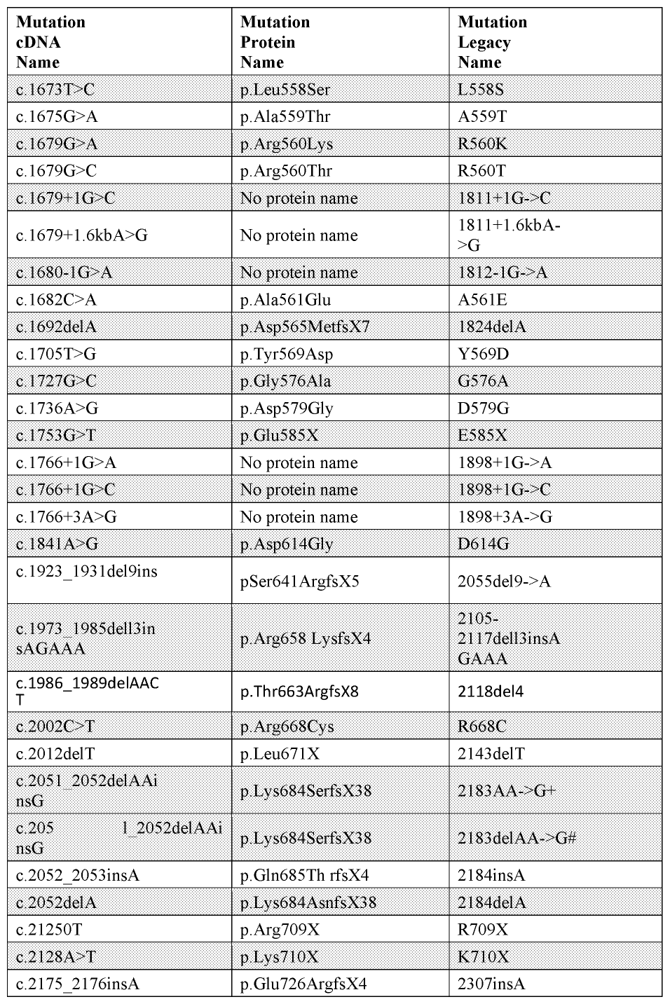

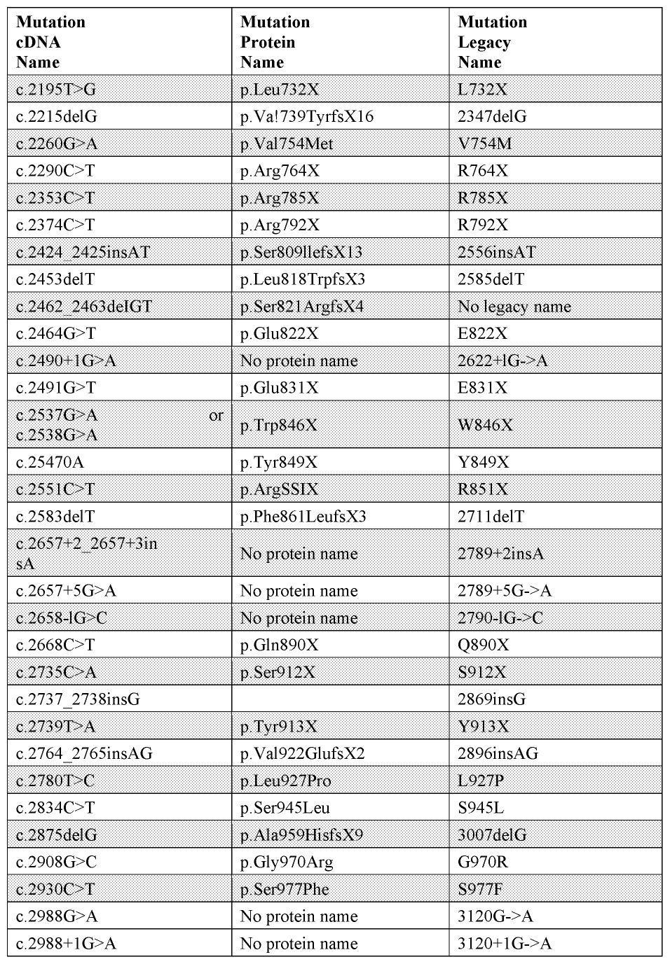

In some embodiments, the CF-causing mutation is selected from Table A. In some embodiments, the CF-causing mutation is selected from Table B. In some embodiments, the CF-causing mutation is selected from Table C. In some embodiments, the CF-causing mutation is selected from FIG. 1. In some embodiments, the patient is heterozygous having a CF-causing mutation on one CFTR allele selected from the mutations listed in the table from FIG. 1 and a CF-causing mutation on the other CFTR allele is selected from the CFTR mutations listed in Table B.

TABLE B

| | | |
|---|---|---|
| Q39X | 405+1G→A | L927P |
| W57X | 405+3A→C | G85E |
| E60X | 406−1G→A | S341P |
| R75X | 621+1G→T | L467P |
| E92X | 1248+1G→A | I507del |
| Q98X | 1341+1G→A | V520F |
| Y122X | 1717−1G→A | A559T |
| L218X | 1811+1.6kbA→G | R560T |
| Q220X | 1811+1G→C | R560S |
| C276X | 1812−1G→A | A561E |
| Q290X | 1898+1G→A | Y569D |
| G330X | 2622+1G→A | L1065P |
| W401X | 3120+1G→A | R1066C |
| Q414X | 3120G→A | R1066M |
| S434X | 3850−1G→A | L1077P |
| S466X | 4005+1G→A | H1085R |
| S489X | 4374+1G→T | M1101K |
| Q493X | 663delT | N1303K |
| W496X | 2183AA→G | 3849+10kbC→T |
| Q525X | CFTRdel2,3 | 3272−26A→G |
| G542X | 3659delC | 711+3A→G |
| Q552X | 394delTT | E56K |
| R553X | 2184insA | P67L |
| E585X | 3905insT | R74W |
| G673X | 2184delA | D110E |
| R709X | 1078delT | D110H |
| K710X | 1154insTC | R117C |
| L732X | 2183delAA→G | L206W |
| R764X | 2143delT | R347H |
| R785X | 1677delTA | R352Q |
| R792X | 3876delA | A455E |
| E822X | 2307insA | D579G |
| W846X | 4382delA | E831X |
| R851X | 4016insT | S945L |
| Q890X | 2347delG | S977F |
| S912X | 3007delG | F1052V |
| W1089X | 574delA | R1070W |
| Y1092X | 2711delT | F1074L |
| E1104X | 3791delC | D1152H |
| R1158X | CFTRdele22-23 | D1270N |
| R1162X | 457TAT→G | G178R |
| S1196X | 2043delG | S549N |
| W1204X | 2869insG | S549R |
| S1196X | 3600+2insT | G551D |
| W1204X | 3737delA | G551S |
| S1255X | 4040delA | G1244E |
| W1282X | 541delC | S1251N |
| Q1313X | A46D | S1255P |
| 621+1G→T | T338I | G1349D |
| 711+1G→T | R347P | |
| 711+5G→A | | |
| 712−1G→T | | |

TABLE C
CFTR Mutations

| Criteria | Mutation | | | | |
|---|---|---|---|---|---|
| Truncation | Q2X | L218X | Q525X | R792X | E1104X |
| mutations | S4X | Q220X | G542X | E822X | W1145X |
| % PI > 50% | W19X | Y275X | G550X | W882X | R1158X |
| and/or | G27X | C276X | Q552X | W846X | R1162X |
| SwCl⁻ > | Q39X | Q290X | R553X | Y849X | S1196X |
| 86 mmol/L | W57X | G330X | E585X | R851X | W1204X |
| No full- | E60X | W401X | G673X | Q890X | L1254X |
| length | R75X | Q414X | Q685X | S912X | S1255X |
| protein | L88X | S434X | R709X | Y913X | W1282X |
| | E92X | S466X | K710X | Q1042X | Q1313X |
| | Q98X | S489X | Q715X | W1089X | Q1330X |
| | Y122X | Q493X | L732X | Y1092X | E1371X |
| | E193X | W496X | R764X | W1098X | Q1382X |
| | W216X | C524X | R785X | R1102X | Q1411X |
| Splice | 185+1G→T | 711+5G→A | 1717−8G→A | 2622+1G→A | 3121−1G→A |
| mutations | 296+1G→A | 712−1G→T | 1717−1G→A | 2790−1G→C | 3500−2A→G |
| % PI > 50% | 296+1G→T | 1248+1G→A | 1811+1G→C | 3040G→C | 3600+2insT |
| and/or | 405+1G→A | 1249−1G→A | 1811+1.6kbA→G | (G970R) | 3850−1G→A |
| SwCl⁻ > | 405+3A→C | 1341+1G→A | 1811+1643G→T | 3120G→A | 4005+1G→A |
| 86 mmol/L | 406−1G→A | 1525−2A→G | 1812−1G→A | 3120+1G→A | 4374+1G→T |
| No or little | 621+1G→T | 1525−1G→A | 1898+1G→A | 3121−2A→G | |
| mature mRNA | 711+1G→T | | 1898+1G→C | | |
| Small (≤3 | 182delT | 1078delT | 1677delTA | 2711delT | 3737delA |
| nucleotide) | 306insA | 1119delA | 1782delA | 2732insA | 3791delC |
| insertion/ | 306delTAGA | 1138insG | 1824delA | 2869insG | 3821delT |
| deletion | 365-366insT | 1154insTC | 1833delT | 2896insAG | 3876delA |
| (ins/del) | 394delTT | 1161delC | 2043delG | 2942insT | 3878delG |
| frameshift | 442delA | 1213delT | 2143delT | 2957delT | 3905insT |
| mutations | 444delA | 1259insA | 2183AA→G^{a} | 3007delG | 4016insT |
| % PI > 50% | 457TAT→G | 1288insTA | 2184delA | 3028delA | 4021dupT |
| and/or | 541delC | 1343delG | 2184insA | 3171delC | 4022insT |
| SwCl⁻ > | 574delA | 1471delA | 2307insA | 3171insC | 4040delA |
| 86 mmol/L | 663delT | 1497delGG | 2347delG | 3271delGG | 4279insA |
| Garbled and/or | 849delG | 1548delG | 2585delT | 3349insT | 4326delTC |
| truncated | 935delA | 1609delCA | 2594delGT | 3659delC | |
| protein | | | | | |

| | | | |
|---|---|---|---|
| Non-small (>3 | CFTRdele1 | CFTRdele16-17b | 1461ins4 |
| nucleotide) | CFTRdele2 | CFTRdele17a,17b | 1924del7 |
| insertion/ | CFTRdele2,3 | CFTRdele17a-18 | 2055del9→A |
| deletion | CFTRdele2-4 | CFTRdele19 | 2105-2117del13insAGAAA |
| (ins/del) | CFTRdele3-10,14b-16 | CFTRdele19-21 | 2372del8 |
| frameshift | CFTRdele4-7 | CFTRdele21 | 2721del11 |
| % PI > 50% | CFTRdele4-11 | CFTRdele22-24 | 2991del32 |
| and/or | CFTR50kbdel | CFTRdele22,23 | 3121-977_3499+248del2515 |
| SwCl⁻ > 86 | CFTRdup6b-10 | 124del23bp | 3667ins4 |
| mmol/L | CFTRdele11 | 602del14 | 4010del4 |
| Garbled and/or | CFTRdele13,14a | 852del22 | 4209TGTT→AA |
| truncated protein | CFTRdele14b-17b | 991del5 | |

| | |
|---|---|
| Criteria | Mutation |

| | | | | |
|---|---|---|---|---|
| Class II, III, IV | A46D^{b} | V520F | Y569D^{b} | N1303K |
| mutations not responsive | G85E | A559T^{b} | L1065P | |
| to Compound II, Compound | R347P | R560T | R1066C | |
| III, or Compound II/ | L467P^{b} | R560S | L1077P^{b} | |
| Compound III | I507del | A561E | M1101K | |
| % PI > 50% and/or | | | | |
| SwCl⁻ > 86 mmol/L AND | | | | |
| Not responsive in | | | | |
| vitro to Compound II, | | | | |
| Compound III, or Compound | | | | |
| II/Compound III | | | | |

CFTR: cystic fibrosis transmembrane conductance regulator; SwCl: sweat chloride
Source: CFTR2.org [Internet]. Baltimore (MD): Clinical and functional translation of CFTR. The Clinical and Functional Translation of CFTR (CFTR2), US Cystic Fibrosis Foundation, Johns Hopkins University, the Hospital for Sick Children. Available at: http://www.cftr2.org/. Accessed 15 Feb. 2016.
Notes:
% PI: percentage of F508del-CFTR heterozygous patients in the CFTR2 patient registry who are pancreatic insufficient; SwCl: mean sweat chloride of F508del-CFTR heterozygous patients in the CFTR2 patient registry.
^{a}Also known as 2183delAA→G.
^{b}Unpublished data.

Table C above includes certain exemplary CFTR minimal function mutations, which are detectable by an FDA-cleared genotyping assay, but does not include an exhaustive list.

In some embodiments, disclosed herein is a method of treating, lessening the severity of, or symptomatically treating cystic fibrosis in a patient with F508del/MF (F/MF) genotypes (heterozygous for F508del and an MF mutation not expected to respond to CFTR modulators, such as Compound III); with F508del/F508del (F/F) genotype (homozygous for F508del); and/or with F508del/gating (F/G) genotypes (heterozygous for F508del and a gating mutation known to be CFTR modulator-responsive (e.g., Compound III-responsive). In some embodiments, a patient with F508del/MF (F/MF) genotypes has a MF mutation that is not expected to respond to Compound II, Compound III, and both of Compound II and Compound III. In some embodiments, a patient with F508del/MF (F/MF) genotypes has any one of the MF mutations in Table C.

In some embodiments, the patient is heterozygous for F508del, and the other CFTR genetic mutation is any CF-causing mutation, including truncation mutations, splice mutations, small (≤3 nucleotide) insertion or deletion (ins/del) frameshift mutations; non-small (>3 nucleotide) insertion or deletion (ins/del) frameshift mutations; and Class II, III, IV mutations not responsive to Compound III alone or in combination with Compound II or Compound IV (lumacaftor).

In some embodiments, the patient is heterozygous for F508del, and the other CFTR genetic mutation is any CF-causing mutation expected to be and/or is responsive to the triple combination of Compound I, Compound II, and Compound III genotypes based on in vitro and/or clinical data.

In some embodiments, the patient is heterozygous for F508del, and the other CFTR genetic mutation is a truncation mutation. In some specific embodiments, the truncation mutation is a truncation mutation listed in Table C.

In some embodiments, the patient is heterozygous for F508del, and the other CFTR genetic mutation is a splice mutation. In some specific embodiments, the splice mutation is a splice mutation listed in Table C.

In some embodiments, the patient is heterozygous for F508del, and the other CFTR genetic mutation is a small (≤3 nucleotide) insertion or deletion (ins/del) frameshift mutation. In some specific embodiments, the small (≤3 nucleotide) insertion or deletion (ins/del) frameshift mutation is a small (≤3 nucleotide) insertion or deletion (ins/del) frameshift mutation listed in Table C.

In some embodiments, the patient is heterozygous for F508del, and the other CFTR genetic mutation is a non-small (>3 nucleotide) insertion or deletion (ins/del) frameshift mutation. In some specific embodiments, the non-small (>3 nucleotide) insertion or deletion (ins/del) frameshift mutation is a non-small (>3 nucleotide) insertion or deletion (ins/del) frameshift mutation listed in Table C.

In some embodiments, the patient is heterozygous for F508del, and the other CFTR genetic mutation is a Class II, III, IV mutations not responsive to Compound III alone or in combination with Compound II. In some specific embodiments, the Class II, III, IV mutations not responsive to Compound III alone or in combination with Compound II is a Class II, III, IV mutations not responsive to Compound III alone or in combination with Compound II or Compound IV (lumacaftor) listed in Table C.

In some embodiments, the patient is heterozygous for F508del, and the other CFTR genetic mutation is any mutation listed in Table C.

In some embodiments, the patient is heterozygous for F508del, and the other CFTR genetic mutation is any mutation listed in Table B, Table C, and FIG. 3.

In some embodiments, the patient is heterozygous for F508del, and the other CFTR genetic mutation is any mutation listed in Table B. In some embodiments, the patient is heterozygous for F508del, and the other CFTR genetic mutation is any mutation listed in Table C. In some embodiments, the patient is heterozygous for F508del, and the other CFTR genetic mutation is any mutation listed in FIG. 3.

In some embodiments, the patient is homozygous for F508del.

In some embodiments, the patient is heterozygous having one CF-causing mutation on one CFTR allele selected from the mutations listed in the table from FIG. 1 and another CF-causing mutation on the other CFTR allele is selected from the CFTR mutations listed in Table C.

Patients with an F508del/gating mutation genotype are defined as patients that are heterozygous F508del-CFTR with a second CFTR allele that contains a mutation associated with a gating defect and clinically demonstrated to be responsive to Compound III. Examples of such mutations include: G178R, S549N, S549R, G551D, G551S, G1244E, S1251N, S1255P, and G1349D.

Patients with an F508del/residual function genotype are defined as patients that are heterozygous F508del-CFTR with a second CFTR allele that contains a mutation that results in reduced protein quantity or function at the cell surface which can produce partial CFTR activity. CFTR gene mutations known to result in a residual function phenotype include in some embodiments, a CFTR residual function mutation selected from 2789+5G→A, 3849+10kbC→T, 3272-26A→G, 711+3A→G, E56K, P67L, R74W, D110E, D110H, R117C, L206W, R347H, R352Q, A455E, D579G, E831X, S945L, S977F, F1052V, R1070W, F1074L, D1152H, D1270N, E193K, and K1060T. In some embodiments, the CFTR residual function mutation is selected from R117H, S1235R, I1027T, R668C, G576A, M470V, L997F, R75Q, R1070Q, R31C, D614G, G1069R, R1162L, E56K, A1067T, E193K, or K1060T. In some embodiments, the CFTR residual function mutation is selected from R117H, S1235R, I1027T, R668C, G576A, M470V, L997F, R75Q, R1070Q, R31C, D614G, G1069R, R1162L, E56K, or A1067T.

In some embodiments, disclosed herein is a method of treating, lessening the severity of, or symptomatically treating cystic fibrosis in a patient comprising administering an effective amount of a pharmaceutical composition of this disclosure to the patient, such as a mammal, wherein the patient possesses a CFTR genetic mutation selected from the mutations listed in FIG. 1.

In some embodiments, the composition disclosed herein is useful for treating, lessening the severity of, or symptomatically treating cystic fibrosis in patients who exhibit residual CFTR activity in the apical membrane of respiratory and non-respiratory epithelia. The presence of residual CFTR activity at the epithelial surface can be readily detected using methods known in the art, e.g., standard electrophysiological, biochemical, or histochemical techniques. Such methods identify CFTR activity using in vivo or ex vivo electrophysiological techniques, measurement of sweat or salivary Cl⁻ concentrations, or ex vivo biochemical or histochemical techniques to monitor cell surface density. Using such methods, residual CFTR activity can be readily detected for patients that are heterozygous or homozygous for a variety of different mutations, including patients heterozygous for the most common mutation, F508del, as well as other mutations such as the G551D mutation, or the R117H mutation. In some embodiments, compositions disclosed herein are useful for treating, lessening the severity of, or symptomatically treating cystic fibrosis in patients who exhibit little to no residual CFTR activity. In some embodiments, compositions disclosed herein are useful for treating, lessening the severity of, or symptomatically treating cystic fibrosis in patients who exhibit little to no residual CFTR activity in the apical membrane of respiratory epithelia.

In some embodiments, the compositions disclosed herein are useful for treating or lessening the severity of cystic fibrosis in patients who exhibit residual CFTR activity using pharmacological methods. Such methods increase the amount of CFTR present at the cell surface, thereby inducing a hitherto absent CFTR activity in a patient or augmenting the existing level of residual CFTR activity in a patient.

In some embodiments, the compositions disclosed herein are useful for treating or lessening the severity of cystic fibrosis in patients with certain genotypes exhibiting residual CFTR activity.

In some embodiments, compositions disclosed herein are useful for treating, lessening the severity of, or symptomatically treating cystic fibrosis in patients within certain clinical phenotypes, e.g., a mild to moderate clinical phenotype that typically correlates with the amount of residual CFTR activity in the apical membrane of epithelia. Such phenotypes include patients exhibiting pancreatic sufficiency.

In some embodiments, the compositions disclosed herein are useful for treating, lessening the severity of, or symptomatically treating patients diagnosed with pancreatic sufficiency, idiopathic pancreatitis and congenital bilateral absence of the vas deferens, or mild lung disease wherein the patient exhibits residual CFTR activity.

In some embodiments, this disclosure relates to a method of augmenting or inducing anion channel activity in vitro or in vivo, comprising contacting the channel with a composition disclosed herein. In some embodiments, the anion channel is a chloride channel or a bicarbonate channel. In some embodiments, the anion channel is a chloride channel.

In some embodiments of the methods of treating cystic fibrosis disclosed herein, the absolute change in the patient's percent predicted forced expiratory volume in one second (ppFEV₁) after 29 days of administration of at least one compound chosen from Compound I and pharmaceutically acceptable salts thereof, at least one compound chosen from Compound II and pharmaceutically acceptable salts thereof, and at least one compound chosen from Compound III and pharmaceutically acceptable salts thereof ranges from 3 percentage points to 40 percentage points relative to the ppFEV1 of the patient prior to said administration.

In some embodiments, the triple combinations are administered to a patient who has one F508del mutation and one minimal function mutation, and who has not taken any of said at least one compound chosen from Compound I and pharmaceutically acceptable salts thereof, at least one compound chosen from Compound II and pharmaceutically acceptable salts thereof, and at least one compound chosen from Compound III and pharmaceutically acceptable salts thereof.

In some embodiments, the triple combinations are administered to a patient has two copies of F508del mutation, and who has taken at least one compound chosen from Compound II and pharmaceutically acceptable salts thereof, and at least one compound chosen from Compound III and pharmaceutically acceptable salts thereof, but not any of said at least one compound chosen from Compound I and pharmaceutically acceptable salts thereof.

In some embodiments, the absolute change in patient's ppFEV₁ after 15 days of administration of at least one compound chosen from Compound I and pharmaceutically acceptable salts thereof, at least one compound chosen from Compound II and pharmaceutically acceptable salts thereof, and at least one compound chosen from Compound III and pharmaceutically acceptable salts thereof ranges from 3 percentage points to 35 percentage points relative to the ppFEV1 of the patient prior to said administration.

In some embodiments, the absolute change in a patient's ppFEV₁ relative to the ppFEV1 of the patient prior to such administration of the triple combinations can be calculated as (postbaseline value-baseline value). The baseline value is defined as the most recent non-missing measurement collected before the first dose of study drug in the Treatment Period (Day 1).

The exact amount of a pharmaceutical composition required will vary from subject to subject, depending on the species, age, and general condition of the subject, the severity of the disease, the particular agent, its mode of administration, and the like. The compounds of this disclosure may be formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of this disclosure will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts. The term “patient”, as used herein, means an animal, such as a mammal, and even further such as a human.

In some embodiments, the disclosure also is directed to methods of treatment using isotope-labelled compounds of the afore-mentioned compounds, which, in some embodiments, are referred to as Compound I′, Compound II′, or Compound III′. In some embodiments, Compound I′, Compound II′, Compound III′, or pharmaceutically acceptable salts thereof, wherein the formula and variables of such compounds and salts are each and independently as described above or any other embodiments described above, provided that one or more atoms therein have been replaced by an atom or atoms having an atomic mass or mass number which differs from the atomic mass or mass number of the atom which usually occurs naturally (isotope labelled). Examples of isotopes which are commercially available and suitable for the disclosure include isotopes of hydrogen, carbon, nitrogen, oxygen, phosphorus, fluorine and chlorine, for example ²H, ³H, ¹³C, ¹⁴C, ¹⁵N, ¹⁸O, ¹⁷O, ³¹F, ³²F, ³⁵S, ¹⁸F and ³⁶Cl respectively.

The isotope-labelled compounds and salts can be used in a number of beneficial ways. They can be suitable for medicaments and/or various types of assays, such as substrate tissue distribution assays. For example, tritium (³H)- and/or carbon-14 (¹⁴C)-labelled compounds are particularly useful for various types of assays, such as substrate tissue distribution assays, due to relatively simple preparation and excellent detectability. For example, deuterium (²H)-labelled ones are therapeutically useful with potential therapeutic advantages over the non-²H-labelled compounds. In general, deuterium (²H)-labelled compounds and salts can have higher metabolic stability as compared to those that are not isotope-labelled owing to the kinetic isotope effect described below. Higher metabolic stability translates directly into an increased in vivo half-life or lower dosages, which could be desired. The isotope-labelled compounds and salts can usually be prepared by carrying out the procedures disclosed in the synthesis schemes and the related description, in the example part and in the preparation part in the present text, replacing a non-isotope-labelled reactant by a readily available isotope-labelled reactant.

In some embodiments, the isotope-labelled compounds and salts are deuterium (²H)-labelled ones. In some specific embodiments, the isotope-labelled compounds and salts are deuterium (²H)-labelled, wherein one or more hydrogen atoms therein have been replaced by deuterium. In chemical structures, deuterium is represented as “D.”

The deuterium (²H)-labelled compounds and salts can manipulate the oxidative metabolism of the compound by way of the primary kinetic isotope effect. The primary kinetic isotope effect is a change of the rate for a chemical reaction that results from exchange of isotopic nuclei, which in turn is caused by the change in ground state energies necessary for covalent bond formation after this isotopic exchange. Exchange of a heavier isotope usually results in a lowering of the ground state energy for a chemical bond and thus causes a reduction in the rate-limiting bond breakage. If the bond breakage occurs in or in the vicinity of a saddle-point region along the coordinate of a multi-product reaction, the product distribution ratios can be altered substantially. For explanation: if deuterium is bonded to a carbon atom at a non-exchangeable position, rate differences of k_{M}/k_{D}=2-7 are typical. For a further discussion, see S. L. Harbeson and R. D. Tung, Deuterium In Drug Discovery and Development, Ann. Rep. Med. Chem. 2011, 46, 403-417, incorporated in its entirety herein by reference.

The concentration of the isotope(s) (e.g., deuterium) incorporated into the isotope-labelled compounds and salt of the disclosure may be defined by the isotopic enrichment factor. The term “isotopic enrichment factor” as used herein means the ratio between the isotopic abundance and the natural abundance of a specified isotope. In some embodiments, if a substituent in a compound of the disclosure is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

When discovering and developing therapeutic agents, the person skilled in the art attempts to optimize pharmacokinetic parameters while retaining desirable in vitro properties. It may be reasonable to assume that many compounds with poor pharmacokinetic profiles are susceptible to oxidative metabolism.

In some embodiments, Compound III′ as used herein includes the deuterated compound disclosed in U.S. Pat. No. 8,865,902 (which is incorporated herein by reference), and CTP-656.

In some embodiments, Compound III′ is:

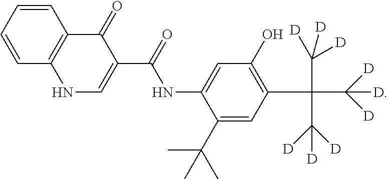

Exemplary embodiments of the disclosure include:

1. A method of treating cystic fibrosis comprising administering to a patient in need thereof:

(A) 400 mg to 1600 mg or 600 mg to 1600 mg of at least one compound chosen from Compound I:

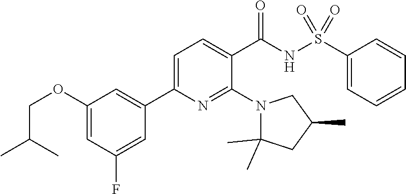 [SMILES: CC(C)COc1cc(F)cc(-c2ccc(C(=O)NS(=O)(=O)c3ccccc3)c(N3C[C@@H](C)CC3(C)C)n2)c1]

and pharmaceutically acceptable salts thereof daily; and

(B) 25 mg to 200 mg of at least one compound chosen from Compound II:

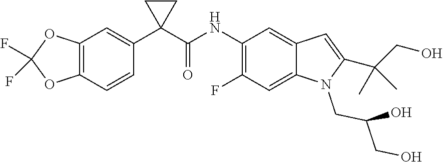 [SMILES: CC(C)(CO)c1cc2cc(NC(=O)C3(c4ccc5c(c4)OC(F)(F)O5)CC3)c(F)cc2n1C[C@@H](O)CO]

and pharmaceutically acceptable salts thereof daily; and

(C) 50 mg to 800 mg of at least one compound chosen from Compound III:

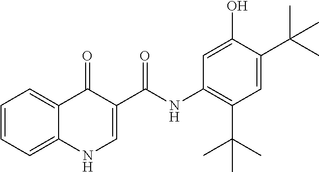 [SMILES: CC(C)(C)c1cc(C(C)(C)C)c(NC(=O)c2c[nH]c3ccccc3c2=O)cc1O]

and pharmaceutically acceptable salts thereof daily.
2. The method according to embodiment 1, wherein 600 mg to 1400 mg, or 1000 mg to 1400 mg of at least one compound chosen from Compound I and pharmaceutically acceptable salts thereof is administered daily.
3. The method according to embodiment 1, wherein 1000 mg to 1200 mg at least one compound chosen from Compound I and pharmaceutically acceptable salts thereof is administered daily.
4. The method according to embodiment 1, wherein 1200 mg to 1600 mg at least one compound chosen from Compound I and pharmaceutically acceptable salts thereof is administered daily.
5. The method according to embodiment 1, wherein 1200 mg to 1400 mg at least one compound chosen from Compound I and pharmaceutically acceptable salts thereof is administered daily.
6. The method according to embodiment 1, wherein 1400 mg to 1600 mg at least one compound chosen from Compound I and pharmaceutically acceptable salts thereof is administered daily.
7. The method according to embodiment 1, wherein 400 mg of at least one compound chosen from Compound I and pharmaceutically acceptable salts thereof is administered daily.
8. The method according to embodiment 1, wherein 1000 mg of at least one compound chosen from Compound I and pharmaceutically acceptable salts thereof is administered daily.
9. The method according to embodiment 1, wherein 1200 mg of at least one compound chosen from Compound I and pharmaceutically acceptable salts thereof is administered daily.
10. The method according to embodiment 1, wherein 1400 mg or 1600 mg of at least one compound chosen from Compound I and pharmaceutically acceptable salts thereof is administered daily.
11. The method according to any one of embodiments 1-10, wherein at least one compound chosen from Compound I and pharmaceutically acceptable salts thereof is administered in a tablet that comprises 100 mg of at least one compound chosen from Compound I and pharmaceutically acceptable salts thereof.
12. The method according to any one of embodiments 1-10, wherein at least one compound chosen from Compound I or a pharmaceutically acceptable salts thereof is administered in a tablet that comprises 150 mg of Compound I or a pharmaceutically acceptable salt thereof.
13. The method according to embodiment 1 or 8, wherein at least one compound chosen from Compound I or a pharmaceutically acceptable salts thereof is administered in a tablet that comprises 200 mg of at least one compound chosen from Compound I and pharmaceutically acceptable salts.
14. The method according to embodiment 1 or 8, wherein at least one compound chosen from Compound I and pharmaceutically acceptable salts thereof is administered in a tablet that comprises 300 mg of at least one compound chosen from Compound I and pharmaceutically acceptable salts thereof.
15. The method according to any one of embodiments 1-14, wherein at least one compound chosen from Compound I and pharmaceutically acceptable salts thereof is administered once daily.
16. The method according to any one of embodiments 1-14, wherein at least one compound chosen from Compound I and pharmaceutically acceptable salts thereof is administered twice daily.
17. The method according to any one of embodiments 1-16, wherein 50 mg to 150 mg of at least one compound chosen from Compound II and pharmaceutically acceptable salts thereof is administered daily.
18. The method according to any one of embodiments 1-16, wherein 75 mg to 200 mg of at least one compound chosen from Compound II and pharmaceutically acceptable salt thereof is administered daily.
19. The method according to any one of embodiments 1-16, wherein 50 mg of at least one compound chosen from Compound II and pharmaceutically acceptable salts thereof is administered daily.
20. The method according to any one of embodiments 1-16, wherein 100 mg of at least one compound chosen from Compound II and pharmaceutically acceptable salts thereof is administered daily.
21. The method according to any one of embodiments 1-20, wherein at least one compound chosen from Compound II and pharmaceutically acceptable salts thereof is administered once daily.
22. The method according to any one of embodiments 1-20, wherein at least one compound chosen from Compound II and pharmaceutically acceptable salts thereof is administered twice daily.
23. The method according to any one of embodiments 1-22, wherein 50 mg to 700 mg at least one compound chosen from Compound III and pharmaceutically acceptable salts thereof is administered daily.
24. The method according to any one of embodiments 1-22, wherein 100 mg to 400 mg at least one compound chosen from Compound III and pharmaceutically acceptable salt thereof is administered daily.
25. The method according to any one of embodiments 1-22, wherein 200 mg to 700 mg at least one compound chosen from Compound III and pharmaceutically acceptable salts thereof is administered daily.
26. The method according to any one of embodiments 1-22, wherein 300 mg to 700 mg at least one compound chosen from Compound III and pharmaceutically acceptable salts thereof is administered daily.
27. The method according to any one of embodiments 1-22, wherein 500 mg to 700 mg at least one compound chosen from Compound III and pharmaceutically acceptable salts thereof is administered daily.
28. The method according to any one of embodiments 1-22, wherein 50 mg daily, 75 mg daily, 100 mg daily, 150 mg daily, 200 mg daily, 300 mg daily or 600 mg of at least one compound chosen from Compound III and pharmaceutically acceptable salts thereof is administered daily.
29. The method according to any one of embodiments 1-28, wherein at least one compound chosen from Compound III and pharmaceutically acceptable salts thereof is administered once daily.
30. The method according to any one of embodiments 1-29, wherein the daily amount of at least one compound chosen from Compound III and pharmaceutically acceptable salts thereof is administered in two doses.
31. The method according to embodiment 1, wherein 50 to 200 mg of at least one compound chosen from Compound II and pharmaceutically acceptable salts thereof is administered daily; and/or 150 mg to 700 mg of at least one compound chosen from Compound III and pharmaceutically acceptable salts thereof is administered daily.
32. The method according to any one of embodiments 1-31, wherein at least one compound chosen from Compound III and pharmaceutically acceptable salts thereof is administered in a single tablet or multiple tablets per dose.
33. The method according to any one of embodiments 1-31, wherein said patient has cystic fibrosis is chosen from patients with F508del/minimal function genotypes, patients with F508del/F508del genotypes, patients with F508del/ gating genotypes, patients with F508del/residual function genotypes, and patients with F508del/ another CFTR genetic mutation expected to be and/or is responsive to the triple combination of Compound I, Compound II, and Compound III genotypes based on in vitro and/or clinical data.
34. A method of treating cystic fibrosis comprising administering to a patient in need thereof a pharmaceutical composition comprising:

(A) 200 mg to 1600 mg or 600 mg to 1600 mg of at least one compound chosen from Compound I:

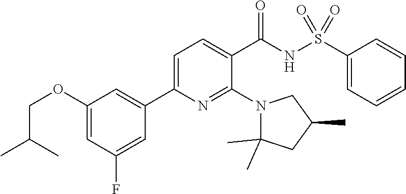 [SMILES: CC(C)COc1cc(F)cc(-c2ccc(C(=O)NS(=O)(=O)c3ccccc3)c(N3C[C@@H](C)CC3(C)C)n2)c1]

and pharmaceutically acceptable salts thereof is administered daily;

(B) 25 mg to 200 mg of at least one compound chosen from Compound II:

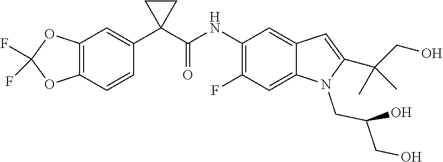 [SMILES: CC(C)(CO)c1cc2cc(NC(=O)C3(c4ccc5c(c4)OC(F)(F)O5)CC3)c(F)cc2n1C[C@@H](O)CO]

and pharmaceutically acceptable salts thereof is administered daily;

(C) 50 mg to 800 mg of at least one compound chosen from Compound III:

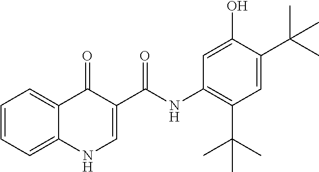 [SMILES: CC(C)(C)c1cc(C(C)(C)C)c(NC(=O)c2c[nH]c3ccccc3c2=O)cc1O]

and pharmaceutically acceptable salts thereof is administered daily; and

(D) a pharmaceutically acceptable carrier.

35. The method according to embodiment 34, wherein 1000 mg to 1400 mg of at least one compound chosen from Compound I and pharmaceutically acceptable salts thereof is administered daily.
36. The method according to embodiment 34, wherein 1000 mg to 1200 mg of at least one compound chosen from Compound I and pharmaceutically acceptable salts thereof is administered daily.
37. The method according to embodiment 34, wherein 1200 mg to 1600 mg of at least one compound chosen from Compound I and pharmaceutically acceptable salts thereof is administered daily.
38. The method according to embodiment 34, wherein 1200 mg to 1400 mg of at least one compound chosen from Compound I and pharmaceutically acceptable salts thereof is administered daily.
39. The method according to embodiment 34, wherein 1400 mg to 1600 mg of at least one compound chosen from Compound I and pharmaceutically acceptable salts thereof is administered daily.
40. The method according to embodiment 34, wherein 400 mg of at least one compound chosen from Compound I and pharmaceutically acceptable salts thereof is administered daily.
41. The method according to embodiment 34, wherein 1000 mg of at least one compound chosen from Compound I and pharmaceutically acceptable salts thereof is administered daily.
42. The method according to embodiment 34, wherein 1200 mg of at least one compound chosen from Compound I and pharmaceutically acceptable salts thereof is administered daily.
43. The method according to embodiment 34, wherein 1400 mg of at least one compound chosen from Compound I and pharmaceutically acceptable salts thereof is administered daily.
44. The method according to any one of embodiments 34-43, wherein at least one compound chosen from Compound I and pharmaceutically acceptable salts thereof is administered in a tablet that comprises 100 mg of Compound I or a pharmaceutically acceptable salt thereof.
45. The method according to any one of embodiments 34-43, wherein at least one compound chosen from Compound I and pharmaceutically acceptable salts thereof is administered in a tablet that comprises 150 mg of Compound I or a pharmaceutically acceptable salt thereof.
46. The method according to any one of embodiments 34 or 41, wherein at least one compound chosen from Compound I and pharmaceutically acceptable salts thereof is administered in a tablet that comprises 200 mg of Compound I or a pharmaceutically acceptable salt thereof.
47. The method according to any one of embodiments 34 or 41, wherein at least one compound chosen from Compound I and pharmaceutically acceptable salts thereof is administered in a tablet that comprises 300 mg of Compound I or a pharmaceutically acceptable salt thereof.
48. The method according to any one of embodiments 34-47, wherein at least one compound chosen from Compound I and pharmaceutically acceptable salts thereof is administered once daily.
49. The method according to any one of embodiments 34-47, wherein at least one compound chosen from Compound I and pharmaceutically acceptable salts thereof is administered twice daily.
50. The method according to any one of embodiments 34-49, wherein 25 mg to 150 mg of at least one compound chosen from Compound II and pharmaceutically acceptable salts thereof is administered daily.
51. The method according to any one of embodiments 34-49, wherein 50 mg to 150 mg of at least one compound chosen from Compound II and pharmaceutically acceptable salts thereof is administered daily.
52. The method according to any one of embodiments 34-49, wherein 75 mg to 200 mg of at least one compound chosen from Compound II and pharmaceutically acceptable salts thereof is administered daily.
53. The method according to any one of embodiments 34-49, wherein 50 mg of at least one compound chosen from Compound II and pharmaceutically acceptable salts thereof is administered daily.
54. The method according to any one of embodiments 34-49, wherein 100 mg of at least one compound chosen from Compound II and pharmaceutically acceptable salts thereof is administered daily.
55. The method according to any one of embodiments 34-54, wherein 50 mg to 700 mg of at least one compound chosen from Compound III and pharmaceutically acceptable salts thereof is administered daily.
56. The method according to any one of embodiments 34-54, wherein 100 mg to 400 mg of at least one compound chosen from Compound III and pharmaceutically acceptable salts thereof is administered daily.
57. The method according to any one of embodiments 34-54, wherein 200 mg to 700 mg of at least one compound chosen from Compound III and pharmaceutically acceptable salts thereof is administered daily.
58. The method according to any one of embodiments 34-54, wherein 300 mg to 700 mg of at least one compound chosen from Compound III and pharmaceutically acceptable salts thereof is administered daily.
59. The method according to any one of embodiments 34-54, wherein 500 mg to 700 mg of at least one compound chosen from Compound III and pharmaceutically acceptable salts thereof is administered daily.
60. The method according to any one of embodiments 34-54, wherein 50 mg, 75 mg, 100 mg, 150 mg, 200 mg, 300 mg, or 600 mg of at least one compound chosen from Compound III and pharmaceutically acceptable salts thereof is administered.
61. The method according to any one of embodiments 34-54, wherein at least one compound chosen from Compound III and pharmaceutically acceptable salts thereof is administered once daily.
62. The method according to any one of embodiments 34-54, wherein at least one compound chosen from Compound III and pharmaceutically acceptable salts thereof is administered twice daily.
63. The method according to embodiment 34, further wherein 50 to 200 mg of at least one compound chosen from Compound II and pharmaceutically acceptable salts thereof is administered daily; and/or 150 mg to 700 mg of at least one compound chosen from Compound III and pharmaceutically acceptable salts thereof is administered daily.
64. The method according to any one of embodiments 34-63, wherein each dose of said pharmaceutical composition in the form of a single tablet or multiple tablets.
65. The method according to any one of embodiments 34-64, wherein said patient has cystic fibrosis is chosen from patients with F508del/minimal function genotypes, patients with F508del/F508del genotypes, patients with F508del/ gating genotypes, patients with F508del/residual function genotypes, and patients with F508del/ another CFTR genetic mutation expected to be and/or is responsive to the triple combination of Compound I, Compound II, and Compound III genotypes based on in vitro and/or clinical data 66. A method of treating cystic fibrosis comprising administering to a patient in need thereof:

(A) a first pharmaceutical composition comprising 400 mg to 1600 mg or 600 mg to 1600 mg of at least one compound chosen from Compound I:

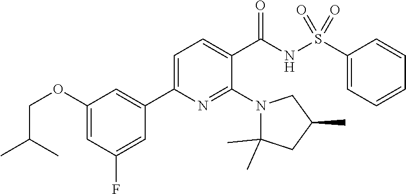 [SMILES: CC(C)COc1cc(F)cc(-c2ccc(C(=O)NS(=O)(=O)c3ccccc3)c(N3C[C@@H](C)CC3(C)C)n2)c1]

and pharmaceutically acceptable salts thereof and a pharmaceutically acceptable carrier daily; and

(B) a second pharmaceutical composition comprising: (i) 25 mg to 200 mg of at least one compound chosen from Compound II:

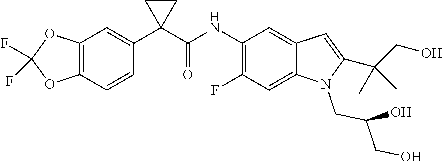 [SMILES: CC(C)(CO)c1cc2cc(NC(=O)C3(c4ccc5c(c4)OC(F)(F)O5)CC3)c(F)cc2n1C[C@@H](O)CO]

and pharmaceutically acceptable salts i and (ii) 50 mg to 800 mg of at least one compound chosen from Compound III:

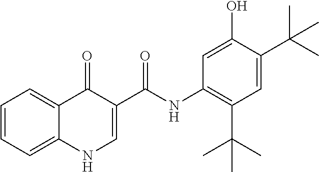 [SMILES: CC(C)(C)c1cc(C(C)(C)C)c(NC(=O)c2c[nH]c3ccccc3c2=O)cc1O]

and pharmaceutically acceptable salts thereof daily.
67. The method according to embodiment 66, wherein 600 mg to 1600 mg or 1000 mg to 1400 mg of at least one compound chosen from Compound I and pharmaceutically acceptable salts thereof is administered daily.
68. The method according to embodiment 66, wherein 1000 mg to 1200 mg of at least one compound chosen from Compound I and pharmaceutically acceptable salts thereof is administered daily.
69. The method according to embodiment 66, wherein 1200 mg to 1600 mg of at least one compound chosen from Compound I and pharmaceutically acceptable salts thereof is administered daily.
70. The method according to embodiment 66, wherein 1200 mg to 1400 mg of at least one compound chosen from Compound I and pharmaceutically acceptable salts thereof is administered daily.
71. The method according to embodiment 66, wherein 1400 mg to 1600 mg of at least one compound chosen from Compound I and pharmaceutically acceptable salts thereof is administered daily.
72. The method according to embodiment 66, wherein 400 mg of at least one compound chosen from Compound I and pharmaceutically acceptable salts thereof is administered daily.
73. The method according to embodiment 66, wherein 1000 mg of at least one compound chosen from Compound I and pharmaceutically acceptable salts thereof is administered daily.
74. The method according to embodiment 66, wherein 1200 mg of at least one compound chosen from Compound I and pharmaceutically acceptable salts thereof is administered daily.
75. The method according to embodiment 66, wherein 1400 mg or 1600 mg of at least one compound chosen from Compound I and pharmaceutically acceptable salts thereof is administered daily.
76. The method according to any one of embodiments 66-75, wherein at least one compound chosen from Compound I and pharmaceutically acceptable salts thereof is administered in a tablet that comprises 100 mg of Compound I or a pharmaceutically acceptable salt thereof.
77. The method according to any one of embodiments 66-75, wherein at least one compound chosen from Compound I and pharmaceutically acceptable salts thereof is administered in a tablet that comprises 150 mg of Compound I or a pharmaceutically acceptable salt thereof.
78. The method according to embodiment 66 or 73, wherein at least one compound chosen from Compound I and pharmaceutically acceptable salts thereof is administered in a tablet that comprises 200 mg of Compound I or a pharmaceutically acceptable salt thereof.
79. The method according to embodiment 66 or 73, wherein at least one compound chosen from Compound I and pharmaceutically acceptable salts thereof is administered in a tablet that comprises 300 mg of Compound I or a pharmaceutically acceptable salt thereof.
80. The method according to any one of embodiments 66-79, wherein at least one compound chosen from Compound I and pharmaceutically acceptable salts thereof is administered once daily.
81. The method according to any one of embodiments 66-79, wherein at least one compound chosen from Compound I and pharmaceutically acceptable salts thereof is administered twice daily.
82. The method according to any one of embodiments 66-81, wherein 50 mg to 150 mg of at least one compound chosen from Compound II and pharmaceutically acceptable salts thereof is administered daily.
83. The method according to any one of embodiments 66-81, wherein 75 mg to 200 mg of at least one compound chosen from Compound II and pharmaceutically acceptable salts thereof is administered daily.
84. The method according to any one of embodiments 66-81, wherein 50 mg of at least one compound chosen from Compound II and pharmaceutically acceptable salts thereof is administered daily.
85. The method according to any one of embodiments 66-81, wherein 100 mg of at least one compound chosen from Compound II and pharmaceutically acceptable salts thereof is administered daily.
86. The method according to any one of embodiments 66-85, wherein at least one compound chosen from Compound II and pharmaceutically acceptable salts thereof is administered once daily.
87. The method according to any one of embodiments 66-85, wherein at least one compound chosen from Compound II and pharmaceutically acceptable salts thereof is administered twice daily.
88. The method according to any one of embodiments 66-87, wherein 50 mg to 700 mg of at least one compound chosen from Compound III and pharmaceutically acceptable salts thereof is administered daily.
89. The method according to any one of embodiments 66-87, wherein 100 mg to 400 mg of at least one compound chosen from Compound III and pharmaceutically acceptable salts thereof is administered daily.
90. The method according to any one of embodiments 66-87, wherein 200 mg to 700 mg of at least one compound chosen from Compound III and pharmaceutically acceptable salts thereof is administered daily.
91. The method according to any one of embodiments 66-87, wherein 300 mg to 700 mg of at least one compound chosen from Compound III and pharmaceutically acceptable salts thereof is administered daily.
92. The method according to any one of embodiments 66-87, wherein 500 mg to 700 mg at least one compound chosen from Compound III and pharmaceutically acceptable salts thereof is administered daily.
93. The method according to any one of embodiments 66-87, wherein 50 mg daily, 75 mg daily, 100 mg daily, 150 mg daily, 200 mg daily, 300 mg daily, or 600 mg daily of at least one compound chosen from Compound III and pharmaceutically acceptable salts thereof is administered daily.
94. The method according to any one of embodiments 66-87, wherein 150 mg or 300 mg of at least one compound chosen from Compound III and pharmaceutically acceptable salts thereof is administered twice daily.
95. The method according to any one of embodiments 66-93, wherein at least one compound chosen from Compound III and pharmaceutically acceptable salts thereof is administered once daily.
96. The method according to any one of embodiments 66-94, wherein the dose of at least one compound chosen from Compound III and pharmaceutically acceptable salts thereof is administered twice daily.
97. The method according to any one of embodiments 66-96, wherein each dose of said first and second pharmaceutical compositions are independently in the form of a single tablet or multiple tablets.
98. The method according to embodiment 66, wherein 50 to 200 mg of at least one compound chosen from Compound II and pharmaceutically acceptable salts thereof is administered daily; and/or 150 mg to 700 mg of at least one compound chosen from Compound III and pharmaceutically acceptable salts thereof is administered daily.
99. The method according to embodiment 66, wherein 150 mg to 700 mg of at least one compound chosen from Compound III and pharmaceutically acceptable salts thereof is administered daily.
100. The method according to any one of embodiments 66-99, wherein said second pharmaceutical composition is administered prior to, subsequent to, or concurrently with said first pharmaceutical composition.
101. The method according to any one of embodiments 66-100, further comprising administering to said patient an additional pharmaceutical composition, said additional pharmaceutical composition comprising at least one compound chosen from Compound II, Compound III, and pharmaceutically acceptable salts thereof.
102. The method according to embodiment 101, wherein said additional pharmaceutical composition is administered once daily.
103. The method according to any one of embodiments 66-102, wherein said patient has cystic fibrosis is chosen from patients with F508del/minimal function genotypes, patients with F508del/F508del genotypes, patients with F508del/gating genotypes, patients with F508del/residual function genotypes, and patients with F508del/ another CFTR genetic mutation expected to be and/or is responsive to the triple combination of Compound I, Compound II, and Compound III genotypes based on in vitro and/or clinical data.
104. The method according to any one of embodiments 1-103, comprising administering to said patient Compound I.
105. The method according to any one of embodiments 1-103, comprising administering to said patient a pharmaceutically acceptable salt of Compound I.
106. The method according to any one of embodiments 1-103, comprising administering to said patient Compound II.
107. The method according to any one of embodiments 1-103, comprising administering to said patient a pharmaceutically acceptable salt of Compound II.
108. The method according to any one of embodiments 1-103, comprising administering to said patient Compound III.
109. The method according to any one of embodiments 1-103, comprising administering to said patient a pharmaceutically acceptable salt of Compound III.
110. The method according to any one of embodiments 1-103, comprising administering to said patient: a pharmaceutically acceptable salt of Compound I; Compound II; and Compound III.
111. The method according to any one of embodiments 1-103, comprising administering to said patient: Compound I; Compound II; and Compound III.
112. The method according to any one of embodiments 1-103, comprising administering to said patient: Compound I; and Compound III.
113. The method according to any one of embodiments 1-103, comprising administering to said patient: a pharmaceutically acceptable salt of Compound I; and Compound III.
114. The method of any one of embodiments 33, 65, or 103, wherein the patient with a F508del/minimal function genotype has a minimal function mutation selected from:

Mutation

| | | | | |
|---|---|---|---|---|
| S4X | C276X | G542X | R792X | E1104X |
| G27X | Q90X | G550X | E822X | R1158X |
| Q39X | G330X | Q552X | W846X | R1162X |
| W57X | W401X | R553X | Y849X | S1196X |
| E60X | Q414X | E585X | R851X | W1204X |
| R75X | S434X | G673X | Q890X | L1254X |
| E92X | S466X | Q685X | S912X | S1255X |
| Q98X | S489X | R709X | Y913X | W1282X |
| Y122X | Q493X | K710X | W1089X | Q1313X |
| E193X | W496X | L732X | Y1092X | E1371X |
| L218X | C524X | R764X | W1098X | Q1382X |
| Q220X | Q525X | R785X | R1102X | Q1411X |
| 185 + 1G → T | 711 + 5G → A | 1717 − 8G → A | 2622 + 1G → A | 3121 − 1G → A |
| 296 + 1G → A | 712 − 1G → T | 1717 − 1G → A | 2490 − 1G → C | 3500 − 2A → G |
| 405 + 1G → A | 1248 + 1G → A | 1811 + 1G → C | 3040G → C | 3600 + 2insT |
| 405 + 3A → C | 1249 − 1G → A | 1811 + 1.6kbA → G | (G970R) | 3850 − 1G → A |
| 406 − 1G → A | 1341 + 1G → A | 1812 − 1G → A | 3120G → A | 4005 + 1G → A |
| 621 + 1G → T | 1525 − 2A → G | 1898 + 1G → A | 3120 + 1G → A | 4374 + 1G → T |
| 711 + 1G → T | 1525 − 1G → A | 1898 + 1G → C | 3121 − 2A → G | |
| 182delT | 1119delA | 1782delA | 2732insA | 3876delA |
| 306insA | 1138insG | 1824delA | 2869insG | 3878delG |
| 365 − 366insT | 1154insTC | 2043delG | 2896insAG | 3905insT |
| 394delTT | 1161delC | 2143delT | 2942insT | 4016insT |
| 442delA | 1213delT | 2183AA → G* | 2957delT | 4021dupT |
| 444delA | 1259insA | 2184delA | 3007delG | 4040delA |
| 457TAT → G | 1288insTA | 2184insA | 3028delA | 4279insA |
| 541delC | 1471delA | 2307insA | 3171delC | 4326delTC |
| 574delA | 1497delGG | 2347delG | 3659delC | |
| 663delT | 1548delG | 2585delT | 3737delA | |
| 935delA | 1609delCA | 2594delGT | 3791delC | |
| 1078delT | 1677delTA | 2711delT | 3821delT | |

| | | |
|---|---|---|
| CFTRdele2,3 | 1461ins4 | 2991del32 |
| CFTRdele22,23 | 1924del7 | 3667ins4 |
| 124del23bp | 2055del9 → A | 4010del4 |
| 852del22 | 2105- | 4209TGTT → AA |
| 991del5 | 2117del13insAGAAA | |
| | 2721del11 | |

Mutation

| | | | |
|---|---|---|---|
| A46D [symbol] | V520F | Y569D [symbol] | N1303K |
| G85E | A559T [symbol] | L1065P | |
| R347P | R560T | R1066C | |
| L467P [symbol] | R560S | L1077P [symbol] | |
| I507del | A561E | M1101K | |

[symbol]

indicates data missing or illegible when filed

115. The method of any one of embodiments 33, 65, or 103, wherein the patient with a F508del/gating genotype has a gating mutation selected from G178R, S549N, S549R, G551D, G551S, G1244E, S1251N, S1255P, and G1349D.
116. The method of any one of embodiments 33, 65, or 103, wherein the patient with a F508del/ residual function genotype has a residual function mutation selected from 2789+5G→A, 3849+10kbC→T, 3272-26A→G, 711+3A→G, E56K, P67L, R74W, D110E, D110H, R117C, L206W, R347H, R352Q, A455E, D579G, E831X, S945L, S977F, F1052V, R1070W, F1074L, D1152H, D1270N, E193K, K1060T, R117H, S1235R, I1027T, R668C, G576A, M470V, L997F, R75Q, R1070Q, R31C, D614G, G1069R, R1162L, E56K, A1067T, E193K, and K1060T.
117. The method according to any one of embodiments 34-116, wherein the pharmaceutically acceptable carrier is HPMCAS-HG.
118. The method according to any one of embodiments 1, 34, or 66, wherein one dose of 400 mg to 1600 mg or 1000 mg to 1600 mg of at least one compound chosen from Compound I and pharmaceutically acceptable salts thereof is administered daily; one dose of 50 to 200 mg of at least one compound chosen from Compound II and pharmaceutically acceptable salts thereof is administered once daily; and one dose of 150 mg to 300 mg of Compound III is administered twice daily.
119. The method according to any one of embodiments 1, 34, or 66, wherein one dose of 400 mg to 1600 mg or 1000 mg to 1600 mg of at least one compound chosen from Compound I and pharmaceutically acceptable salts thereof is administered once daily; one dose of 100 mg of at least one compound chosen from Compound II and pharmaceutically acceptable salts thereof is administered once daily; and one dose of 150 mg or 300 mg of at least one compound chosen from Compound III and pharmaceutically acceptable salts thereof is administered twice daily.
120. The method according to any one of embodiments 1, 34, or 66, wherein one dose of 200 mg to 800 mg or 500 mg to 800 mg of at least one compound chosen from Compound I and pharmaceutically acceptable salts thereof is administered twice daily; one dose of 50 mg of at least one compound chosen from Compound II and pharmaceutically acceptable salts thereof is administered twice daily; and one dose of 150 mg or 300 mg of at least one compound chosen from Compound III and pharmaceutically acceptable salts thereof is administered twice daily.
121. The method according to any one of embodiments 1, 34, or 66, wherein one dose of 400 mg to 1600 mg or 1000 mg to 1600 mg of at least one compound chosen from Compound I and pharmaceutically acceptable salts thereof is administered once daily; and one dose of 150 mg to 300 mg of Compound III or a pharmaceutically acceptable salt is administered twice daily.
122. The method according to any one of embodiments 1, 34, or 66, wherein one dose of 400 mg to 800 mg or 500 mg to 800 mg of at least one compound chosen from Compound I and pharmaceutically acceptable salts thereof is administered twice daily; and one dose of 150 mg or 300 mg of at least one compound chosen from Compound III and pharmaceutically acceptable salts thereof is administered twice daily.
123. The method according to any one of claim 1, 34, or 66, wherein one dose of 400 mg, 1000 mg, 1200 mg, 1400 mg, or 1600 mg of at least one compound chosen from Compound I and pharmaceutically acceptable salts thereof is administered daily.
124. The method according to any one of claim 1, 34, or 66, wherein one dose of 200 mg or 600 mg of at least one compound chosen from Compound I and pharmaceutically acceptable salts thereof is administered twice daily.
125. The method according to any one of claim 1, 34, or 66, wherein one dose of 400 mg, 1000 mg, 1200 mg, 1400 mg, or 1600 mg of at least one compound chosen from Compound I and pharmaceutically acceptable salts thereof is administered daily; one dose of 100 mg of at least one compound chosen from Compound II and pharmaceutically acceptable salts thereof is administered once daily; and one dose of 150 mg or 300 mg of at least one compound chosen from Compound III and pharmaceutically acceptable salts thereof is administered twice daily.
126. The method according to any one of embodiments 1, 34, or 66, wherein one dose of 600 mg of at least one compound chosen from Compound I and pharmaceutically acceptable salts thereof is administered twice daily; one dose of 100 mg of Compound II is administered once daily; and one dose of 150 mg or 300 mg of Compound III is administered twice daily.
127. The method according to any one of embodiments 1, 34, or 66, wherein one dose of 600 mg of at least one compound chosen from Compound I and pharmaceutically acceptable salts thereof is administered twice daily; one dose of 50 mg of Compound II is administered twice daily; and one dose of 300 mg of Compound III is administered twice daily.
128. A single tablet comprising a first solid dispersion, a second solid dispersion, and a third solid dispersion,
(a) wherein the first solid dispersion comprises 50 mg to 800 mg of Compound I

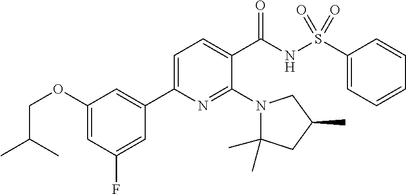 [SMILES: CC(C)COc1cc(F)cc(-c2ccc(C(=O)NS(=O)(=O)c3ccccc3)c(N3C[C@@H](C)CC3(C)C)n2)c1]

and 10 wt % to 60 wt % of a polymer relative to the total weight of the first solid dispersion;

(b) wherein the second solid dispersion comprises 3 mg to 70 mg of Compound II:

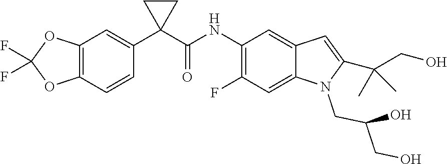 [SMILES: CC(C)(CO)c1cc2cc(NC(=O)C3(c4ccc5c(c4)OC(F)(F)O5)CC3)c(F)cc2n1C[C@@H](O)CO]

and 10 wt % to 30 wt % of a polymer relative to the total weight of the second solid dispersion; and
(c) wherein the third solid dispersion comprises 10 mg to 400 mg of Compound III:

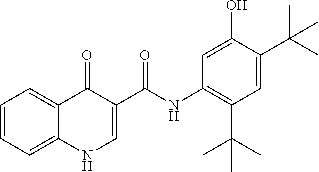 [SMILES: CC(C)(C)c1cc(C(C)(C)C)c(NC(=O)c2c[nH]c3ccccc3c2=O)cc1O]

and 10 wt % to 30 wt % of a polymer relative to the total weight of the third solid dispersion.
129. The single tablet of embodiment 128, wherein the polymer in the first solid dispersion is present in 10 wt % to 50 wt %, 10 wt % to 40 wt %, or 10 wt % to 30 wt %, relative to the total weight of the first solid dispersion.
130. The single tablet of embodiment 128, wherein the polymer in the first solid dispersion is present in 15 wt % to 25 wt % relative to the total weight of the first solid dispersion.
131. The single tablet of embodiment 128, wherein the polymer in the first solid dispersion is present in 20 wt % relative to the total weight of the first solid dispersion.
132. The single tablet of any one of embodiments 128-131, wherein at least one of the first, second, and third solid dispersions is a spray-dried dispersion.
133. The single tablet of any one of embodiments 128-131, wherein each of the first, second, and third solid dispersions is a spray-dried dispersion.
134. The single tablet of any one of embodiments 128-133, wherein each of said polymers in the first solid dispersion, second solid dispersion, and third solid dispersion comprises one or more polymers independently selected from cellulose-based polymers, polyoxyethylene-based polymers, polyethylene-propylene glycol copolymers, vinyl-based polymers, PEO-polyvinyl caprolactam-based polymers, and polymethacrylate-based polymers.
135. The single tablet of embodiment 134:

wherein the cellulose-based polymer is selected from a methylcellulose, a hydroxypropyl methylcellulose (hypromellose), a hypromellose phthalate (HPMC-P), and a hypromellose acetate succinate;

wherein the polyoxyethylene-based polymer or polyethylene-propylene glycol copolymer is selected from a polyethylene glycol and a poloxamer;

wherein the vinyl-based polymer is a polyvinylpyrrolidine;

wherein the PEO-polyvinyl caprolactam-based polymer is a polyethylene glycol, polyvinyl acetate and polyvinylcaprolactam-based graft copolymer; and

wherein the polymethacrylate-based polymer is a poly(methacrylic acid, ethyl acrylate) (1:1) or a dimethylaminoethyl methacrylate-methylmethacrylate copolymer.

136. The single tablet of embodiment 135, wherein the cellulose-based polymer is a hypromellose acetate succinate and a hypromellose, or a combination of hypromellose acetate succinate and a hypromellose.
137. The single tablet of embodiment 136, wherein the cellulose-based polymer is selected from hypromellose E15, hypromellose acetate succinate L, and hypromellose acetate succinate H.
138. The single tablet of embodiment 136, wherein the polyoxyethylene-based polymer or polyethylene-propylene glycol copolymer is selected from polyethylene glycol 3350 and poloxamer 407.
139. The single tablet of embodiment 136, wherein the vinyl-based polymer is selected from polyvinylpyrrolidine K30 and polyvinylpyrrolidine VA 64.
140. The single tablet of embodiment 136, wherein the polymethacrylate polymer is selected from Eudragit L100-55 and Eudragit E PO.
141. The single tablet of embodiment 134, wherein said polymer for the first solid dispersion is selected from the group consisting of a hypromellose acetate succinate and a hypromellose, and a combination thereof; said polymer for the second solid dispersion is a hypromellose acetate succinate; and said polymer for the third solid dispersion is a hypromellose acetate succinate.
142. The single tablet of embodiment 134, wherein said polymer for the first solid dispersion is a hypromellose acetate succinate; said polymer for the second solid dispersion is hypromellose; and said polymer for the third solid dispersion is a hypromellose acetate succinate.
143. The single tablet of embodiment 134, wherein said polymer for the first solid dispersion is selected from the group consisting of hydroxypropyl methylcellulose (HPMC) E15, hypromellose acetate succinate L, hypromellose acetate succinate H, and a combination thereof; said polymer for the second solid dispersion is HPMC E15; and said polymer for the third solid dispersion is hypromellose acetate succinate H.
144. The single tablet of embodiment 134, wherein said polymer for the first solid dispersion is hypromellose acetate succinate H; said polymer for the second solid dispersion is HPMC E15; and said polymer for the third solid dispersion is hypromellose acetate succinate H.
145. The single tablet of embodiment 134, wherein said polymer for the first solid dispersion is hypromellose acetate succinate HG; said polymer for the second solid dispersion is HPMC E15; and said polymer for the third solid dispersion is hypromellose acetate succinate HG.
146. The single tablet of any one of embodiments 128-145, wherein the first solid dispersion comprises 50 mg to 600 mg of Compound I.
147. The single tablet of any one of embodiments 128-145, wherein the first solid dispersion comprises 50 mg to 400 mg, 50 mg to 300 mg, 50 mg, 75 mg, 100 mg, 150 mg, 200 mg, or 300 mg of Compound I.
148. The single tablet of any one of embodiments 128-145, wherein the first solid dispersion comprises 200 mg of Compound I.
149. The single tablet of any one of embodiments 128-145, wherein the first solid dispersion comprises 300 mg of Compound I.
150. The single tablet of any one of embodiments 128-149, wherein the second solid dispersion comprises 5 mg to 50 mg of Compound II.
151. The single tablet of any one of embodiments 128-149, wherein the second solid dispersion comprises 5 mg to 35 mg of Compound II.
152. The single tablet of any one of embodiments 128-149, wherein the second solid dispersion comprises 5 mg to 10 mg, 10 mg to 20 mg, or 20 mg to 30 mg of Compound II.
153. The single tablet of any one of embodiments 128-149, wherein the third solid dispersion comprises 15 mg to 200 mg of Compound III.
154. The single tablet of any one of embodiments 128-149, wherein the third solid dispersion comprises 15 mg to 50 mg, 25 mg to 75 mg, 50 mg to 100 mg, 75 mg to 125 mg, or 125 mg to 175 mg of Compound III.
155. The single tablet of any one of embodiments 128-145, wherein:

the first solid dispersion comprises 50 mg to 150 mg of Compound I:

the second solid dispersion comprises 3 mg to 15 mg of Compound II: and

the third solid dispersion comprises 10 mg to 75 mg of Compound.

156. The single tablet of any one of embodiments 128-145, wherein:

the first solid dispersion comprises 150 mg to 250 mg of Compound I:

the second solid dispersion comprises 10 mg to 25 mg of Compound II: and

the third solid dispersion comprises 30 mg to 125 mg of Compound.

157. The single tablet of any one of embodiments 128-145, wherein:

the first solid dispersion comprises 250 mg to 350 mg of Compound I:

the second solid dispersion comprises 15 mg to 30 mg of Compound II: and

the third solid dispersion comprises 75 mg to 175 mg of Compound III.

158. The single tablet of any one of embodiments 128-145, wherein Compounds I, II, and III are in a weight ratio of Compound I:Compound II:Compound III 10 to 15:1:5 to 7.
159. The single tablet of any one of embodiments 128-145, wherein Compounds I, II, and III are in a weight ratio of Compound I:Compound II:Compound III 12:1:3 to 6.
160. The single tablet of any one of embodiments 128-159, comprising one or more excipients selected from a filler, a disintegrant, a surfactant, and a lubricant.
161. The single tablet of embodiment 160, wherein the filler is selected from microcrystalline cellulose, silicified microcrystalline cellulose, lactose, dicalcium phosphate, mannitol, copovidone, hydroxypropyl cellulose, hypromellose, methyl cellulose, ethyl cellulose, starch, Maltodextrin, agar, guar gum, and pullulan.
162. The single tablet of embodiment 160, wherein the disintegrant is selected from croscarmellose sodium, sodium starch glycolate, crospovidone, corn or pre-gelatinized starch, sodium carboxymethyl cellulose, calcium carboxymethyl cellulose, and microcrystalline cellulose.
163. The single tablet of embodiment 160, wherein the lubricant is selected from magnesium stearate, sodium stearyl fumarate, calcium stearate, sodium stearate, stearic acid, and talc; and wherein the surfactant is selected from sodium lauryl sulfate, poloxamers, docusate sodium, PEGs and PEG derivatives.
164. The single tablet of any one of embodiments 128-163, wherein each of Compounds I, II and III is independently substantially amorphous.
165. A single tablet comprising:

(a) 30 wt % to 50 wt % of a first solid dispersion relative to the total weight of the tablet;

(b) 1 wt % to 8 wt % of a second solid dispersion relative to the total weight of the tablet; and

(c) 10 wt % to 35 wt % of a third solid dispersion relative to the total weight of the tablet;

wherein the first solid dispersion comprises 40 wt % to 90 wt % of Compound I

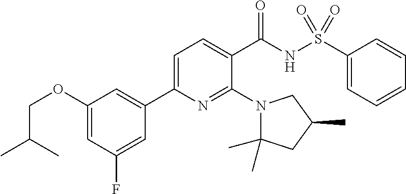 [SMILES: CC(C)COc1cc(F)cc(-c2ccc(C(=O)NS(=O)(=O)c3ccccc3)c(N3C[C@@H](C)CC3(C)C)n2)c1]

and 10 wt % to 60 wt % of a polymer relative to the total weight of the first solid dispersion;

wherein the second solid dispersion comprises 70 wt % to 90 wt % of Compound II:

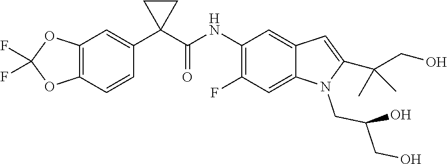 [SMILES: CC(C)(CO)c1cc2cc(NC(=O)C3(c4ccc5c(c4)OC(F)(F)O5)CC3)c(F)cc2n1C[C@@H](O)CO]

and 10 wt % to 30 wt % of a polymer relative to the total weight of the second solid dispersion; and

wherein the third solid dispersion comprises 70 wt % to 90 wt % of Compound III:

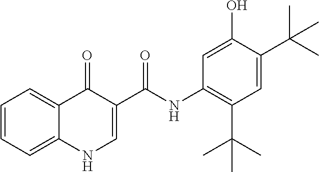 [SMILES: CC(C)(C)c1cc(C(C)(C)C)c(NC(=O)c2c[nH]c3ccccc3c2=O)cc1O]

and 10 wt % to 30 wt % of a polymer relative to the total weight of the third solid dispersion.

166. The single tablet of embodiment 165, wherein the polymer in the first solid dispersion is present in 10 wt % to 50 wt %, 10 wt % to 40 wt %, or 10 wt % to 30 wt % relative to the total weight of the first solid dispersion.
167. The single tablet of embodiment 165, wherein the polymer in the first solid dispersion is present in 15 wt % to 25 wt % relative to the total weight of the first solid dispersion.
168. The single tablet of embodiment 165, wherein the polymer in the first solid dispersion is present in 20 wt % relative to the total weight of the first solid dispersion.
169. The single tablet of any one of embodiments 165-168, wherein at least one of the first, second, and third solid dispersions is a spray-dried dispersion.
170. The single tablet of any one of embodiments 165-168, wherein each of the first, second, and third solid dispersions is a spray-dried dispersion.
171. The single tablet of any one of embodiments 165-170, wherein each of said polymers in the first solid dispersion, second solid dispersion, and third solid dispersion comprises one or more polymers independently selected from cellulose-based polymers, polyoxyethylene-based polymers, polyethylene-propylene glycol copolymers, vinyl-based polymers, PEO-polyvinyl caprolactam-based polymers, and polymethacrylate-based polymers.
172. The single tablet of embodiment 171,

wherein the cellulose-based polymer is selected from a

methylcellulose, a hydroxypropyl methylcellulose (hypromellose), a hypromellose phthalate (HPMC-P), and a hypromellose acetate succinate;

wherein the polyoxyethylene-based polymer or polyethylene-propylene glycol copolymer is selected from a polyethylene glycol and a poloxamer;

wherein the vinyl-based polymer is a polyvinylpyrrolidine;

wherein the PEO-polyvinyl caprolactam-based polymer is a polyethylene glycol, polyvinyl acetate and polyvinylcaprolactam-based graft copolymer; and

wherein the polymethacrylate-based polymer is a poly(methacrylic acid, ethyl acrylate) (1:1) or a dimethylaminoethyl methacrylate-methylmethacrylate copolymer.

173. The single tablet of embodiment 172, wherein the cellulose-based polymer is a hypromellose acetate succinate and a hypromellose, or a combinations of hypromellose acetate succinate and a hypromellose.
174. The single tablet of embodiment 172, wherein the cellulose-based polymer is selected from hypromellose E15, hypromellose acetate succinate L and hypromellose acetate succinate H.
175. The single tablet of embodiment 172, wherein the polyoxyethylene-based polymer or polyethylene-propylene glycol copolymer is selected from polyethylene glycol 3350 and poloxamer 407.
176. The single tablet of embodiment 171, wherein the vinyl-based polymer is selected from polyvinylpyrrolidine K30 and polyvinylpyrrolidine VA 64.
177. The single tablet of embodiment 171, wherein the polymethacrylate polymer is selected from Eudragit L100-55 and Eudragit E PO.
178. The single tablet of embodiment 171, wherein said polymer for the first solid dispersion is selected from the group consisting of a hypromellose acetate succinate and a hypromellose, and a combination thereof; said polymer for the second solid dispersion is a hypromellose acetate succinate; and said polymer for the third solid dispersion is a hypromellose acetate succinate.
179. The single tablet of embodiment 171, wherein said polymer for the first solid dispersion is a hypromellose acetate succinate; said polymer for the second solid dispersion is hypromellose; and said polymer for the third solid dispersion is a hypromellose acetate succinate.
180. The single tablet of embodiment 171, wherein said polymer for the first solid dispersion is selected from the group consisting of hydroxypropyl methylcellulose E15, hypromellose acetate succinate L, hypromellose acetate succinate H, and a combination thereof; said polymer for the second solid dispersion is hypromellose (HPMC E15); and said polymer for the third solid dispersion is hypromellose acetate succinate H.
181. The single tablet of embodiment 165, wherein:

the second solid dispersion comprises 70 wt % to 85 wt % of Compound II relative to the total weight of the second solid dispersion, and the polymer is hydroxypropyl methylcellulose in an amount of 15 wt % to 30 wt % relative to the total weight of the second solid dispersion; and

the third solid dispersion comprises 70 wt % to 85 wt % of Compound III relative to the total weight of the third solid dispersion, and the polymer is hypromellose acetate succinate in an amount of 15 wt % to 30 wt % relative to the total weight of the second solid dispersion.

182. The single tablet of embodiment 165, wherein:

the second solid dispersion comprises 70 wt % to 85 wt % of Compound II relative to the total weight of the second solid dispersion, and the polymer is hydroxypropyl methylcellulose in an amount of 15 wt % to 30 wt % relative to the total weight of the second solid dispersion; and

the third solid dispersion comprises 80 wt % of Compound III relative to the total weight of the third solid dispersion, and the polymer is hypromellose acetate succinate in an amount of 15 wt % to 20 wt % relative to the total weight of the second solid dispersion.

183. The single tablet of any one of embodiments 165-182, wherein the first solid dispersion comprises 50 wt % to 90 wt % of Compound I.
184. The single tablet of any one of embodiments 165-182, wherein the first solid dispersion comprises 60 wt % to 90 wt % of Compound I.
185. The single tablet of any one of embodiments 165-182, wherein the first solid dispersion comprises 70 wt % to 90 wt % of Compound I.
186. The single tablet of any one of embodiments 165-182, wherein the first solid dispersion comprises 75 wt % to 85 wt % of Compound I.
187. The single tablet of any one of embodiments 165-182, wherein the first solid dispersion comprises 80 wt % of Compound I.
188. The single tablet of any one of embodiments 165-187, wherein the second solid dispersion comprises 75 wt % to 85 wt % of Compound II.
189. The single tablet of any one of embodiments 165-187, wherein the second solid dispersion comprises 80 wt % of Compound II.
190. The single tablet of any one of embodiments 165-189, wherein the third solid dispersion comprises 75 wt % to 85 wt % of Compound III.
191. The single tablet of any one of embodiments 165-189, wherein the third solid dispersion comprises 80 wt % of Compound III.
192. The single tablet of any one of embodiments 165-191, comprising one or more excipients selected from a filler, a disintegrant, a surfactant, and a lubricant.
193. The single tablet of embodiment 192, wherein the filler is selected from microcrystalline cellulose, silicified microcrystalline cellulose, lactose, dicalcium phosphate, mannitol, copovidone, hydroxypropyl cellulose, hypromellose, methyl cellulose, ethyl cellulose, starch, Maltodextrin, agar, guar gum, and pullulan.
194. The single tablet of embodiment 192, wherein the disintegrant is selected from croscarmellose sodium, sodium starch glycolate, crospovidone, corn or pre-gelatinized starch, sodium carboxymethyl cellulose, calcium carboxymethyl cellulose, and microcrystalline cellulose.
195. The single tablet of embodiment 192, wherein the lubricant is selected from magnesium stearate, sodium stearyl fumarate, calcium stearate, sodium stearate, stearic acid, and talc, and wherein the surfactant is selected from sodium lauryl sulfate, poloxamers, docusate sodium, PEGs and PEG derivatives.
196. The single tablet of any one of embodiments 165-195 wherein each of Compounds I, II and III is independently substantially amorphous.
197. A single tablet comprising a solid dispersion comprising 50 mg to 800 mg of Compound I:

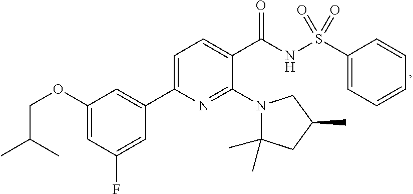 [SMILES: CC(C)COc1cc(F)cc(-c2ccc(C(=O)NS(=O)(=O)c3ccccc3)c(N3C[C@@H](C)CC3(C)C)n2)c1]

### 3 mg to 70 mg of Compound II:

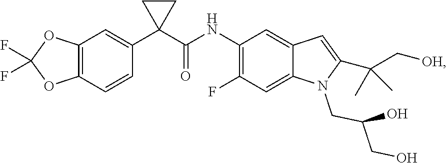 [SMILES: CC(C)(CO)c1cc2cc(NC(=O)C3(c4ccc5c(c4)OC(F)(F)O5)CC3)c(F)cc2n1C[C@@H](O)CO]

### 10 mg to 400 mg of Compound III:

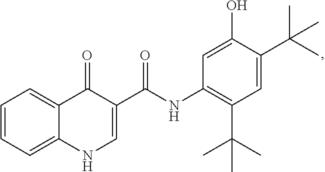 [SMILES: CC(C)(C)c1cc(C(C)(C)C)c(NC(=O)c2c[nH]c3ccccc3c2=O)cc1O]

and
one or more polymers.
198. The single tablet of embodiment 198, wherein the polymer in the solid dispersion is present in 10 wt % to 50 wt % relative to the total weight of the solid dispersion.
199. The single tablet of embodiment 198, wherein the polymer in the solid dispersion is present in 10 wt % to 40 wt % relative to the total weight of the solid dispersion.
200. The single tablet of embodiment 198, wherein the polymer in the solid dispersion is present in 10 wt % to 30 wt % relative to the total weight of the solid dispersion.
201. The single tablet of embodiment 198, wherein the polymer in the solid dispersion is present in 15 wt % to 25 wt % relative to the total weight of the solid dispersion.
202. The single tablet of embodiment 198, wherein the polymer in the solid dispersion is present in 20 wt % relative to the total weight of the solid dispersion.
203. The single tablet of any one of embodiments 198-202, wherein the polymer is one or more polymers selected from cellulose-based polymers, polyoxyethylene-based polymers, polyethylene-propylene glycol copolymers, vinyl-based polymers, PEO-polyvinyl caprolactam-based polymers, and polymethacrylate-based polymers.
204. The single tablet of embodiment 203, wherein the cellulose-based polymer is selected from a methylcellulose, a hydroxypropyl methylcellulose (hypromellose), a hypromellose phthalate (HPMC-P), and a hypromellose acetate succinate;

wherein the polyoxyethylene-based polymer or polyethylene-propylene glycol copolymer is selected from a polyethylene glycol and a poloxamer;

wherein the vinyl-based polymer is a polyvinylpyrrolidine;

wherein the PEO-polyvinyl caprolactam-based polymer is a polyethylene glycol, polyvinyl acetate and polyvinylcaprolactam-based graft copolymer; and

wherein the polymethacrylate-based polymer is an a poly(methacrylic acid, ethyl acrylate) (1:1) or a dimethylaminoethyl methacrylate-methylmethacrylate copolymer.

205. The single tablet of embodiment 204, wherein the cellulose-based polymer is selected from hypromellose acetate succinate and a hypromellose, or a combinations of hypromellose acetate succinate and a hypromellose.
206. The single tablet of embodiment 204, wherein the polyoxyethylene-based polymer or polyethylene-propylene glycol copolymer is selected from polyethylene glycol 3350 and poloxamer 407.
207. The single tablet of embodiment 204, wherein the vinyl-based polymer is selected from polyvinylpyrrolidine K30 and polyvinylpyrrolidine VA 64.
208. The single tablet of embodiment 204, wherein the polymethacrylate polymer is selected from Eudragit L100-55 and Eudragit E PO.
209. The single tablet of any one of embodiments 198-202, wherein the tablet comprises a cellulose-based polymer.
210. The single tablet of any one of embodiments 198-202, wherein the tablet comprises a hypromellose acetate succinate.
211. The single tablet of any one of embodiments 198-202, wherein the tablet comprises a hypromellose.
212. The single tablet of any one of embodiments 198-202, wherein the tablet comprises one or more polymers selected from hydroxypropyl methylcellulose E15, hypromellose acetate succinate L, and hypromellose acetate succinate H.
213. The single tablet of any one of embodiments 197-212, wherein the solid dispersion comprises 50 mg to 400 mg of Compound I.
214. The single tablet of any one of embodiments 197-212, wherein the solid dispersion comprises 50 mg to 300 mg of Compound I.
215. The single tablet of any one of embodiments 197-212, wherein the solid dispersion comprises 50 mg, 75 mg, 100 mg, 150 mg, 200 mg, or 300 mg of Compound I.
216. The single tablet of any one of embodiments 197-212, wherein the solid dispersion comprises 200 mg of Compound I.
217. The single tablet of any one of embodiments 197-212, wherein the solid dispersion comprises 300 mg of Compound I.
218. The single tablet of any one of embodiments 197-217, wherein the solid dispersion comprises 5 mg to 50 mg of Compound II.
219. The single tablet of any one of embodiments 197-217, wherein the solid dispersion comprises 5 mg to 35 mg of Compound II.
220. The single tablet of any one of embodiments 197-217, wherein the solid dispersion comprises 5 mg to 10 mg, 10 mg to 20 mg, or 20 mg to 30 mg of Compound II.
221. The single tablet of any one of embodiments 197-220, wherein the solid dispersion comprises 15 mg to 200 mg of Compound III.
222. The single tablet of any one of embodiments 197-220, wherein the solid dispersion comprises 15 mg to 50 mg, 25 mg to 75 mg, 50 mg to 100 mg, 75 mg to 125 mg, or 125 mg to 175 mg of Compound III
223. The single tablet of any one of embodiments 197-222, wherein:

the first solid dispersion comprises 50 mg to 150 mg of Compound I:

the second solid dispersion comprises 3 mg to 15 mg of Compound II: and

the third solid dispersion comprises 10 mg to 75 mg of Compound III.

224. The single tablet of any one of embodiments 197-222, wherein:

the first solid dispersion comprises 150 mg to 250 mg of Compound I:

the second solid dispersion comprises 10 mg to 25 mg of Compound II: and

the third solid dispersion comprises 30 mg to 125 mg of Compound III.

225. The single tablet of any one of embodiments 197-222, wherein:

the first solid dispersion comprises 250 mg to 350 mg of Compound I:

the second solid dispersion comprises 15 mg to 30 mg of Compound II: and

the third solid dispersion comprises 75 mg to 175 mg of Compound III.

226. The single tablet of any one of embodiments 197-222, wherein Compounds I, II, and III are in a weight ratio of Compound I:Compound II:Compound III 10:1:5 to 7.
227. The single tablet of any one of embodiments 197-222, wherein Compounds I, II, and III are in a weight ratio of Compound I:Compound II:Compound III 12:1:3 to 6.
228. The single tablet of any one of embodiments 197-227, comprising one or more excipients selected from a filler, a disintegrant, a surfactant, and a lubricant.
229. The single tablet of embodiment 228, wherein the filler is selected from microcrystalline cellulose, silicified microcrystalline cellulose, lactose, dicalcium phosphate, mannitol, copovidone, hydroxypropyl cellulose, hypromellose, methyl cellulose, ethyl cellulose, starch, Maltodextrin, agar, guar gum, and pullulan.
230. The single tablet of embodiment 228, wherein the disintegrant is selected from croscarmellose sodium, sodium starch glycolate, crospovidone, corn or pre-gelatinized starch, sodium carboxymethyl cellulose, calcium carboxymethyl cellulose, and microcrystalline cellulose.
231. The single tablet of embodiment 228, wherein the lubricant is selected from magnesium stearate, sodium stearyl fumarate, calcium stearate, sodium stearate, stearic acid, and talc; and wherein the surfactant is selected from sodium lauryl sulfate, poloxamers, docusate sodium, PEGs and PEG derivatives.
232. The single tablet of any one of embodiments 197-231, wherein each of Compounds I, II and III is independently substantially amorphous.
233. A single tablet comprising 40 wt % to 90 wt % of a solid dispersion relative to the total weight of the tablet, wherein the solid dispersion comprises

15 wt % to 50 wt % of Compound I relative to the total weight of the solid dispersion;

1 wt % to 5 wt % of Compound II relative to the total weight of the solid dispersion; and

3 wt % to 25 wt % of Compound III relative to the total weight of the solid dispersion.

234. The single tablet of embodiment 233, wherein the solid dispersion is present in 50 wt % to 80 wt % relative to the total weight of the tablet.
235. The single tablet of embodiment 233, wherein the solid dispersion is present in 60 wt % to 70 wt % relative to the total weight of the tablet.
236. The single tablet of any one of embodiments 233-235, wherein the polymer in the solid dispersion is one or more polymers selected from cellulose-based polymers, polyoxyethylene-based polymers, polyethylene-propylene glycol copolymers vinyl-based polymers, PEO-polyvinyl caprolactam-based polymers, and polymethacrylate-based polymers.
237. The single tablet of embodiment 236, wherein the cellulose-based polymer is selected from a methylcellulose, a hydroxypropyl methylcellulose (hypromellose), a hypromellose phthalate (HPMC-P), and a hypromellose acetate succinate;

wherein the polyoxyethylene-based polymer or polyethylene-propylene glycol copolymer is selected from a polyethylene glycol and a poloxamer;

wherein the vinyl-based polymer is a polyvinylpyrrolidine;

wherein the PEO-polyvinyl caprolactam-based polymer is a polyethylene glycol, polyvinyl acetate and polyvinylcaprolactam-based graft copolymer; and

wherein the polymethacrylate-based polymer is an a poly(methacrylic acid, ethyl acrylate) (1:1) or a dimethylaminoethyl methacrylate-methylmethacrylate copolymer.

238. The single tablet of embodiment 237, wherein the cellulose-based polymer is a hypromellose acetate succinate and a hypromellose, or a combinations of hypromellose acetate succinate and a hypromellose.
239. The single tablet of embodiment 237, wherein the polyoxyethylene-based polymer or polyethylene-propylene glycol copolymer is selected from polyethylene glycol 3350 and poloxamer 407.
240. The single tablet of embodiment 237, wherein the vinyl-based polymer is selected from polyvinylpyrrolidine K30 and polyvinylpyrrolidine VA 64.
241. The single tablet of embodiment 237, wherein the polymethacrylate polymer is selected from Eudragit L100-55 and Eudragit E PO.
242. The single tablet of any one of embodiments 233-235, wherein the tablet comprises a cellulose-based polymer.
243. The single tablet of any one of embodiments 233-235, wherein the tablet comprises a hypromellose acetate succinate.
244. The single tablet of any one of embodiments 233-235, wherein the tablet comprises a hypromellose.
245. The single tablet of any one of embodiments 233-235, wherein the table comprises one or more polymers selected from hydroxypropyl methylcellulose E15, hypromellose acetate succinate L, and hypromellose acetate succinate H.
246. The single tablet of any one of embodiments 233-245, comprising one or more excipients selected from a filler, a disintegrant, a surfactant and a lubricant.
247. The single tablet of embodiment 246, wherein the filler is selected from microcrystalline cellulose, silicified microcrystalline cellulose, lactose, dicalcium phosphate, mannitol, copovidone, hydroxypropyl cellulose, hypromellose, methyl cellulose, ethyl cellulose, starch, Maltodextrin, agar, guar gum, and pullulan.
248. The single tablet of embodiment 246, wherein the disintegrant is selected from croscarmellose sodium, sodium starch glycolate, crospovidone, corn or pre-gelatinized starch, sodium carboxymethyl cellulose, calcium carboxymethyl cellulose, and microcrystalline cellulose
249. The single tablet of embodiment 246, wherein the lubricant is selected from magnesium stearate, sodium stearyl fumarate, calcium stearate, sodium stearate, stearic acid, and talc; and wherein the surfactant is selected from sodium lauryl sulfate, poloxamers, docusate sodium, PEGs and PEG derivatives.
250. The single tablet of any one of embodiments 233-249, wherein each of Compounds I, II, and III is independently substantially amorphous.
251. The single tablet of embodiment 197 comprising an intra-granular part and extra-granular part,
(a) wherein the intra-granular part comprises:

the solid dispersion comprising said Compound I, II and III in total in 65 wt % to 75 wt % relative to the total weight of the tablet;

a filler and/or binder in 20 wt % to 25 wt % relative to the total weight of the tablet;

a disintegrant in 1 wt % to 5 wt % relative to the total weight of the tablet; and

a lubricant in 0.5 wt % to 1.5 wt % relative to the total weight of the tablet; and

(b) wherein the extra-granular part comprises: a disintegrant in 1 wt % to 3 wt % relative to the total weight of the tablet, and a lubricant in 1 wt % to 3 wt % relative to the total weight of the tablet.
251a. The single tablet of embodiment 197 comprising:

the solid dispersion comprising said Compound I, II and III in total in 65 wt % to 75 wt % relative to the total weight of the tablet;

a filler and/or binder in 20 wt % to 25 wt % relative to the total weight of the tablet;

a disintegrant in 2 wt % to 8 wt % relative to the total weight of the tablet; and

a lubricant in 1.5 wt % to 4.5 wt % relative to the total weight of the tablet.

252. The single tablet of embodiment 165 comprising an intra-granular part and extra-granular part, (a) wherein the intra-granular part comprises:

the first solid dispersion comprising said Compound I in 40 wt % to 50 wt % relative to the total weight of the tablet;

the second solid dispersion comprising said Compound II in 1 wt % to 5 wt % relative to the total weight of the tablet;

a filler and/or binder in 20 wt % to 25 wt % relative to the total weight of the tablet;

a disintegrant in 1 wt % to 5 wt % relative to the total weight of the tablet; and

a lubricant in 0.5 wt % to 1.5 wt % relative to the total weight of the tablet; and

(b) wherein the extra-granular part comprises:

the third solid dispersion comprising said Compound III in 15 wt % to 25 wt % relative to the total weight of the tablet;

a disintegrant in 1 wt % to 3 wt % relative to the total weight of the tablet, and a lubricant in 1 wt % to 3 wt % relative to the total weight of the tablet.
252a. The single tablet of embodiment 165 comprising:

the first solid dispersion comprising said Compound I in 40 wt % to 50 wt % relative to the total weight of the tablet;

the second solid dispersion comprising said Compound II in 1 wt % to 5 wt % relative to the total weight of the tablet;

a filler and/or binder in 20 wt % to 25 wt % relative to the total weight of the tablet;

a disintegrant in 2 wt % to 8 wt % relative to the total weight of the tablet;

a lubricant in 1.5 wt % to 4.5 wt % relative to the total weight of the tablet; and

the third solid dispersion comprising said Compound III in 15 wt % to 25 wt % relative to the total weight of the tablet.

253. The single tablet of embodiment 165 comprising an intra-granular part and extra-granular part,

(a) wherein the intra-granular part comprises:

the first solid dispersion comprising said Compound I in 40 wt % to 50 wt % relative to the total weight of the tablet;

the second solid dispersion comprising said Compound II in 1 wt % to 5 wt % relative to the total weight of the tablet;

the third solid dispersion comprising said Compound III in 15 wt % to 25 wt % relative to the total weight of the tablet; and

a disintegrant in 1 wt % to 8 wt % relative to the total weight of the tablet; and

(b) wherein the extra-granular part comprises:

a filler and/or binder in 20 wt % to 25 wt % relative to the total weight of the tablet; and

a lubricant in 0.5 wt % to 1.5 wt % relative to the total weight of the tablet.

253a. The single tablet of embodiment 165 comprising:

the first solid dispersion comprising said Compound I in 40 wt % to 50 wt % relative to the total weight of the tablet;

the second solid dispersion comprising said Compound II in 1 wt % to 5 wt % relative to the total weight of the tablet;

the third solid dispersion comprising said Compound III in 15 wt % to 25 wt % relative to the total weight of the tablet;

a disintegrant in 1 wt % to 8 wt % relative to the total weight of the tablet;

a filler and/or binder in 20 wt % to 25 wt % relative to the total weight of the tablet; and

a lubricant in 0.5 wt % to 1.5 wt % relative to the total weight of the tablet.

254. The single tablet of embodiment 165 comprising an intra-granular part and extra-granular part, (a) wherein the intra-granular part comprises:

the first solid dispersion comprising said Compound I in 40 wt % to 50 wt % relative to the total weight of the tablet;

the second solid dispersion comprising said Compound II in 1 wt % to 5 wt % relative to the total weight of the tablet;

the third solid dispersion comprising said Compound III in 15 wt % to 25 wt % relative to the total weight of the tablet; and

a filler and/or binder in 20 wt % to 25 wt % relative to the total weight of the tablet; and

(b) wherein the extra-granular part comprises:

a disintegrant in 1 wt % to 8 wt % relative to the total weight of the tablet; and

a lubricant in 0.5 wt % to 1.5 wt % relative to the total weight of the tablet.

254a. The single tablet of embodiment 165 comprising:

the first solid dispersion comprising said Compound I in 40 wt % to 50 wt % relative to the total weight of the tablet;

the second solid dispersion comprising said Compound II in 1 wt % to 5 wt % relative to the total weight of the tablet;

the third solid dispersion comprising said Compound III in 15 wt % to 25 wt % relative to the total weight of the tablet;

a filler and/or binder in 20 wt % to 25 wt % relative to the total weight of the tablet;

a disintegrant in 1 wt % to 8 wt % relative to the total weight of the tablet; and

a lubricant in 0.5 wt % to 1.5 wt % relative to the total weight of the tablet.

255. The single tablet of embodiment 165 comprising an intra-granular part and extra-granular part,
(a) wherein the intra-granular part comprises:

the first solid dispersion comprising said Compound I in 40 wt % to 50 wt % relative to the total weight of the tablet;

the second solid dispersion comprising said Compound II in 1 wt % to 5 wt % relative to the total weight of the tablet;

the third solid dispersion comprising said Compound III in 15 wt % to 25 wt % relative to the total weight of the tablet; and

a disintegrant in 1 wt % to 8 wt % relative to the total weight of the tablet;

(b) wherein the extra-granular part comprises:

a filler and/or binder in 20 wt % to 25 wt % relative to the total weight of the tablet; and

a lubricant in 0.5 wt % to 1.5 wt % relative to the total weight of the tablet.

255a. The single tablet of embodiment 165 comprising:

the first solid dispersion comprising said Compound I in 40 wt % to 50 wt % relative to the total weight of the tablet;

the second solid dispersion comprising said Compound II in 1 wt % to 5 wt % relative to the total weight of the tablet;

the third solid dispersion comprising said Compound III in 15 wt % to 25 wt % relative to the total weight of the tablet;

a disintegrant in 1 wt % to 8 wt % relative to the total weight of the tablet;

a filler and/or binder in 20 wt % to 25 wt % relative to the total weight of the tablet; and

a lubricant in 0.5 wt % to 1.5 wt % relative to the total weight of the tablet.

256. The single tablet of embodiment 165 comprising a first intra-granular part, a second intra-granular part, and extra-granular part,
(a) wherein the first intra-granular part comprises:

the first solid dispersion comprising said Compound I in 40 wt % to 50 wt % relative to the total weight of the tablet;

a filler and/or binder in 20 wt % to 25 wt % relative to the total weight of the tablet; and

a disintegrant in 1 wt % to 5 wt % relative to the total weight of the tablet; and

(b) wherein the second intra-granular part comprises:

the second solid dispersion comprising said Compound II in 1 wt % to 5 wt % relative to the total weight of the tablet;

the third solid dispersion comprising said Compound III in 15 wt % to 25 wt % relative to the total weight of the tablet;

a filler and/or binder in 1 wt % to 10 wt % relative to the total weight of the tablet; and

a disintegrant in 1 wt % to 3 wt % relative to the total weight of the tablet;

(c) wherein the extra-granular part comprises:

a filler and/or binder in 10 wt % to 15 wt % relative to the total weight of the tablet;

a disintegrant in 1 wt % to 3 wt % relative to the total weight of the tablet; and

a lubricant in 0.5 wt % to 1.5 wt % relative to the total weight of the tablet.

256a. The single tablet of embodiment 165 comprising:

the first solid dispersion comprising said Compound I in 40 wt % to 50 wt % relative to the total weight of the tablet;

a filler and/or binder in 20 wt % to 25 wt % relative to the total weight of the tablet; and

a disintegrant in 1 wt % to 5 wt % relative to the total weight of the tablet;

the second solid dispersion comprising said Compound II in 1 wt % to 5 wt % relative to the total weight of the tablet;

the third solid dispersion comprising said Compound III in 15 wt % to 25 wt % relative to the total weight of the tablet;

a filler and/or binder in 10 wt % to 25 wt % relative to the total weight of the tablet; and

a disintegrant in 2 wt % to 6 wt % relative to the total weight of the tablet; and

a lubricant in 0.5 wt % to 1.5 wt % relative to the total weight of the tablet.

257. A method of treating cystic fibrosis in a patient comprising orally administering to the patient the single tablet of any one of embodiments 128-256, and 251a, 252a, 253a, 254a, 255a, and 256a.
258. The method of embodiment 257, wherein the single tablet is administered once daily.
259. The method of embodiment 257, wherein the single tablet is administered twice daily.
260. The method of embodiment 257, wherein two tablets are administered two times daily.
261. The method of embodiment 257, wherein three tablets are administered two times daily.
262. The method according to any one of embodiments 257-261, wherein said patient has cystic fibrosis is chosen from patients with F508del/minimal function genotypes, patients with F508del/F508del genotypes, patients with F508del/ gating genotypes, patients with F508del/residual function genotypes, and patients with F508del/ another CFTR genetic mutation expected to be and/or is responsive to the triple combination of Compound I, Compound II, and Compound III genotypes based on in vitro and/or clinical data.
263. The method of embodiment 262, wherein the patient with a F508del/minimal function genotype has a minimal function mutation selected from:

Mutation

| S4X | C276X | G542X | R792X | E1104X | | |
|---|---|---|---|---|---|---|
| G27X | Q290X | G550X | E822X | R11 | [symbol] | 8X |

| Q39X | G330X | Q552X | W846X | | | R1162X |
|---|---|---|---|---|---|---|
| W57X | W401X | R553X | Y849X | | | S1196X |
| E60X | Q414X | E585X | R851X | | | W1204X |
| R75X | S434X | G673X | Q890X | | | L1254 |
| E92X | S466X | Q685X | S912X | | | S1255X |
| Q98X | S489X | R709X | Y913X | | | W1282X |
| Y122X | Q493X | K710X | W1089X | | | Q1313X |
| E193X | W496X | L732X | Y1092X | | | R1371X |
| L218X | C524X | R764X | W1098X | | | Q1382X |
| Q220X | Q525X | R785X | R1102X | | | Q1411X |
| 185+1G→T | 711+5G→A | 1717−8G→A | 2622+1G→A | | | 3121−1G→A |
| 296+1G→A | 721−1G→T | 1717−1G→AA | 2790−1G→C | | | 3500−2A→G |
| 405+1G→A | 1248+1G→A | 1811+1G→C | 3040G→C | | | 3600+2insT |
| 405+3A→C | 1249−1G→A | 1811+1.6kbA→G | (G970 | [symbol] | ) | 3850−1G→A |

| | | | | |
|---|---|---|---|---|
| 406−1G→A | 1341+1G→A | 1812−1G→A | 3120G→A | 4005+1G→A |
| 621+1G→T | 1525−2A→G | 1898+1G→A | 3120+1G→A | 4374+1G→T |
| 711+1G→T | 1525−1G→A | 1898+1G→C | 3121−2A→G | |
| 182delT | 1119delA | 1782delA | 2732insA | 3876delA |
| 306insA | 1138insG | 1824delA | 2865insG | 3878delG |
| 365-366insT | 1154insTC | 2043delG | 2896insAG | 3905insT |
| 394delTT | 1161delC | 2143delT | 2942insT | 4016insT |
| 442delA | 1213delT | 2183AA→G [symbol] | 2957delT | 4021dupT |
| 444delA | 1259insA | 2184delA | 3007delG | 4040delA |
| 457TAT→G | 1288insTA | 2184insA | 3028delA | 4279insA |
| 541delC | 1471delA | 2307insA | 3171delC | 4326delTC |
| 574delA | 1497delGG | 2347delG | 3659delC | |
| 663delT | 1548delG | 2585delT | 3737delA | |
| 935delA | 1609del CA | 2594delGT | 3791delC | |
| 1078delT | 1677delTA | 2711delT | 3821delT | |

| | | |
|---|---|---|
| CFTRdele2,3 | 1461ins4 | 2991del32 |
| CFTRdele22,23 | 1924del7 | 3199del6 [symbol] |
| 124del23bp | 2055del9→A | 3667ins4 |
| 852del22 | 2105-2117del13insAGAAA | 4010del4 |
| 991del5 | 2721del11 | 4209TGTT→AA |

[symbol]

indicates data missing or illegible when filed

264. The method of embodiment 262, wherein the patient with a F508del/ gating genotype has a gating mutation selected from G178R, S549N, S549R, G551D, G551S, G1244E, S1251N, S1255P, and G1349D.
265. The method of embodiment 262, wherein the patient with a F508del/ residual function genotype has a residual function mutation selected from 2789+5G→A, 3849+10kbC→T, 3272-26A→G, 711+3A→G, E56K, P67L, R74W, D110E, D110H, R117C, L206W, R347H, R352Q, A455E, D579G, E831X, S945L, S977F, F1052V, R1070W, F1074L, D1152H, D1270N, E193K, K1060T, R117H, S1235R, I1027T, R668C, G576A, M470V, L997F, R75Q, R1070Q, R31C, D614G, G1069R, R1162L, E56K, A1067T, E193K, and K1060T.
266. A pharmaceutical composition comprising

a solid dispersion comprising: (a) Compound I

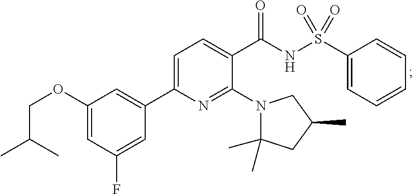 [SMILES: CC(C)COc1cc(F)cc(-c2ccc(C(=O)NS(=O)(=O)c3ccccc3)c(N3C[C@@H](C)CC3(C)C)n2)c1]

and (b) a polymer; and

a pharmaceutically acceptable carrier.

267. The pharmaceutical composition of embodiment 266, wherein the solid dispersion comprises 1-90 wt % polymer.
268. The pharmaceutical composition of embodiment 266, wherein the solid dispersion comprises 5-50 wt % polymer.
269. The pharmaceutical composition of embodiment 266, wherein the solid dispersion comprises 5-40 wt % polymer.
270. The pharmaceutical composition of embodiment 266, wherein the solid dispersion comprises 5-30 wt % polymer.
271. The pharmaceutical composition of embodiment 266, wherein the solid dispersion comprises 5-25 wt % polymer.
272. The pharmaceutical composition of embodiment 266, wherein the solid dispersion comprises 10-25 wt % polymer.
273. The pharmaceutical composition of embodiment 266, wherein the solid dispersion comprises 15-25 wt % polymer.
274. The pharmaceutical composition of embodiment 266, wherein the solid dispersion comprises 20 wt % polymer.
275. The pharmaceutical composition of any one of embodiments 266-274, wherein the spray-dried dispersion comprises one or more polymers selected independently from cellulose-based polymers, polyoxyethylene-based polymers, polyethylene-propylene glycol copolymers, vinyl-based polymers, PEO-polyvinyl caprolactam-based polymers, and polymethacrylate-based polymers.
276. The pharmaceutical composition of embodiment 275, wherein the cellulose-based polymer is a hypromellose acetate succinate and a hypromellose, or a combinations of hypromellose acetate succinate and a hypromellose.
277. The pharmaceutical composition of embodiment 275, wherein the cellulose-based polymer is selected from hypromellose E15, hypromellose acetate succinate L, and hypromellose acetate succinate H.
278. The pharmaceutical composition of embodiment 275, wherein the polyoxyethylene-based polymer or polyethylene-propylene glycol copolymer is selected from polyethylene glycol 3350 and poloxamer 407.
279. The pharmaceutical composition of embodiment 275, wherein the vinyl-based polymer is selected from polyvinylpyrrolidine K30 and polyvinylpyrrolidine VA 64.
280. The pharmaceutical composition of embodiment 275, wherein the PEO-polyvinyl caprolactam-based polymer is a polyethylene glycol, polyvinyl acetate and polyvinylcaprolactam-based graft copolymer.
281. The pharmaceutical composition of embodiment 275, wherein the polyelectrolyte-based polymer is a poly(methacrylic acid, ethyl acrylate) (1:1) or a dimethylaminoethyl methacrylate-methylmethacrylate copolymer.
282. The pharmaceutical composition of embodiment 275, wherein the polyelectrolyte-based polymer is selected from Eudragit L100-55 and Eudragit E PO.
283. The pharmaceutical composition of any one of embodiments 266-274, wherein the solid dispersion comprises hypromellose acetate succinate L or hypromellose acetate succinate H.
284. The pharmaceutical composition of any one of embodiments 251-256, wherein Compounds I, II, and III are in a weight ratio of Compound I:Compound II:Compound III 10 to 15:1:5 to 7.
285. A method of treating cystic fibrosis comprising administering to a patient in need thereof:

(A) 200 mg of at least one compound chosen from Compound I and pharmaceutically acceptable salts thereof twice daily:

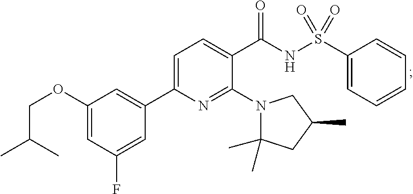 [SMILES: CC(C)COc1cc(F)cc(-c2ccc(C(=O)NS(=O)(=O)c3ccccc3)c(N3C[C@@H](C)CC3(C)C)n2)c1]

(B) 100 mg of at least one compound chosen from Compound II and pharmaceutically acceptable salts thereof once daily:

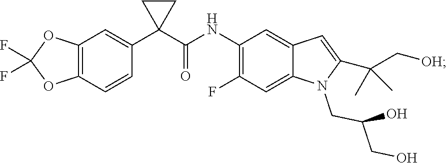 [SMILES: CC(C)(CO)c1cc2cc(NC(=O)C3(c4ccc5c(c4)OC(F)(F)O5)CC3)c(F)cc2n1C[C@@H](O)CO]

and

(C) 150 mg of at least one compound chosen from Compound III and pharmaceutically acceptable salts thereof twice daily:

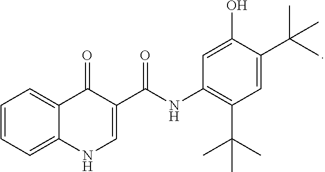 [SMILES: CC(C)(C)c1cc(C(C)(C)C)c(NC(=O)c2c[nH]c3ccccc3c2=O)cc1O]

286. A method of treating cystic fibrosis comprising administering to a patient in need thereof:

(A) 200 mg of at least one compound chosen from Compound I and pharmaceutically acceptable salts thereof twice daily:

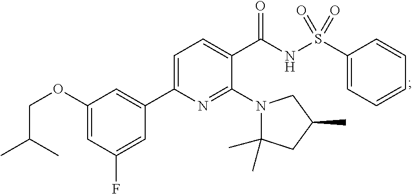 [SMILES: CC(C)COc1cc(F)cc(-c2ccc(C(=O)NS(=O)(=O)c3ccccc3)c(N3C[C@@H](C)CC3(C)C)n2)c1]

(B) 50 mg of at least one compound chosen from Compound II and pharmaceutically acceptable salts thereof twice daily:

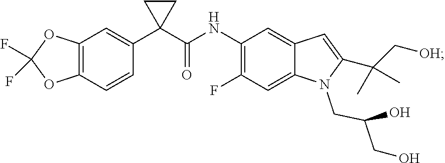 [SMILES: CC(C)(CO)c1cc2cc(NC(=O)C3(c4ccc5c(c4)OC(F)(F)O5)CC3)c(F)cc2n1C[C@@H](O)CO]

and

(C) 150 mg of at least one compound chosen from Compound III and pharmaceutically acceptable salts thereof twice daily:

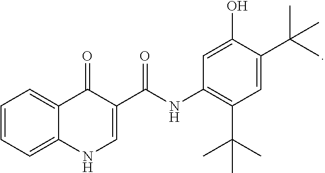 [SMILES: CC(C)(C)c1cc(C(C)(C)C)c(NC(=O)c2c[nH]c3ccccc3c2=O)cc1O]

287. A method of treating cystic fibrosis comprising administering to a patient in need thereof:

(A) 600 mg of at least one compound chosen from Compound I and pharmaceutically acceptable salts thereof twice daily:

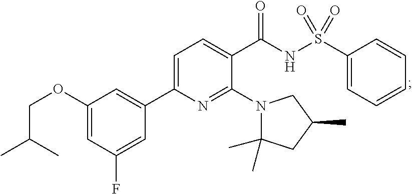 [SMILES: CC(C)COc1cc(F)cc(-c2ccc(C(=O)NS(=O)(=O)c3ccccc3)c(N3C[C@@H](C)CC3(C)C)n2)c1]

(B) 50 mg of at least one compound chosen from Compound II and pharmaceutically acceptable salts thereof twice daily:

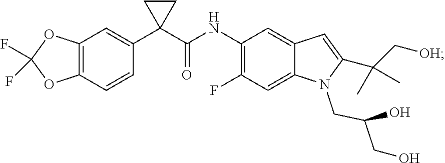 [SMILES: CC(C)(CO)c1cc2cc(NC(=O)C3(c4ccc5c(c4)OC(F)(F)O5)CC3)c(F)cc2n1C[C@@H](O)CO]

and

(C) 300 mg of at least one compound chosen from Compound III and pharmaceutically acceptable salts thereof twice daily:

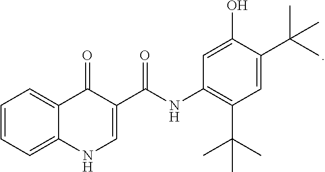 [SMILES: CC(C)(C)c1cc(C(C)(C)C)c(NC(=O)c2c[nH]c3ccccc3c2=O)cc1O]

288. The method according to any one of embodiments 285-287, wherein said patient has cystic fibrosis is chosen from patients with F508del/minimal function genotypes, patients with F508del/F508del genotypes, patients with F508del/ gating genotypes, patients with F508del/residual function genotypes, and patients with F508del/ another CFTR genetic mutation that is expected to be and/or is responsive to the triple combination of Compound I, Compound II, and/or Compound III genotypes based on in vitro and/or clinical data.
289. The method of any one of embodiments 285-287, wherein the patient with a F508del/minimal function genotype has a minimal function mutation selected, from:

Mutation

| | | | | |
|---|---|---|---|---|
| S4X | C276X | G542X | R792X | E1104X |
| G27X | Q290X | G550X | E822X | R1158X |
| Q39X | G330X | Q552X | W846X | R1162X |
| W57X | W401X | R553X | Y849X | S1196X |
| E60X | Q414X | E585X | R851X | W1204X |
| R75X | S434X | G673X | Q890X | L1254X |
| E92X | S466X | Q685X | S912X | S1255X |
| Q98X | S489X | R709X | Y913X | W1282X |
| Y122X | Q493X | K710X | W1089X | Q1313X |
| E193X | W496X | L732X | Y1092X | E1371X |
| L218X | C524X | R764X | W1098X | Q1382X |
| Q220X | Q525X | R785X | R1102X | Q1411X |
| 185 + 1G → T | 711 + 5G → A | 1717 − 8G → A | 2622 + 1G → A | 3121 − 1G → A |
| 296 + 1G → A | 712 − 1G → T | 1717 − 1G → A | 2790 − 1G → C | 3500 − 2A → G |
| 405 + 1G → A | 1248 + 1G → A | 1811 + 1G → C | 3040G → C | 3600 + 2insT |
| 405 + 3A → C | 1249 − 1G → A | 1811 + 1.6kbA → G | (G970R) | 3850 − 1G → A |
| 406 − 1G → A | 1341 + 1G → A | 1812 − 1G → A | 3120G → A | 4005 + 1G → A |
| 621 + 1G → T | 1525 − 2A → G | 1898 + 1G → A | 3120 + 1G → A | 4374 + 1G → T |
| 711 + 1G → T | 1525 − 1G → A | 1898 + 1G → C | 3121 − 2A → G | |
| 182delT | 1119delA | 1782delA | 2732insA | 3876delA |
| 306insA | 1138insG | 1824delA | 2869insG | 3878delG |
| 365-366insT | 1154insTC | 2043delG | 2896insAG | 3905insT |
| 394delTT | 1161delC | 2143delT | 2942insT | 4016insT |
| 442delA | 1213delT | 2183AA → G [symbol] | 2957delT | 4021dupT |
| 444delA | 1259insA | 2184delA | 3007delG | 4040delA |
| 457TAT → G | 1288insTA | 2184insA | 3028delA | 4279insA |
| 541delC | 1471delA | 2307insA | 3171delC | 4326delTC |
| 574delA | 1497delGG | 2347delG | 3659delC | |
| 663delT | 1548delG | 2585delT | 3737delA | |
| 935delA | 1609del CA | 2594delGT | 3791delC | |
| 1078delT | 1677delTA | 2711delT | 3821delT | |
| CFTRdele2,3 | 1461ins4 | 2991del32 | | |
| CFTRdele22,23 | 1924del7 | 3199del6 [symbol] | | |
| 124del23bp | 2055del9 → A | 3667ins4 | | |
| 852del22 | 2105-2117del13insAGAAA | 4010del4 | | |
| 991del5 | 2721del11 | 4209TGTT → AA | | |
| A46D^{b} | V520F | Y569D^{b} | N1303K | |
| G85E | A559T^{b} | L1065P | | |
| R347P | R560T | R1066C | | |
| L467P^{b} | R560S | L1077P^{b} | | |
| I507del | A561E | M1101K | | |

[symbol]

indicates data missing or illegible when filed

290. The method according to any one of embodiments 285-289, wherein the absolute change in said patient's percent predicted forced expiratory volume in one second (ppFEV₁) after 29 days of administration of at least one compound chosen from Compound I and pharmaceutically acceptable salts thereof, at least one compound chosen from Compound II and pharmaceutically acceptable salts thereof, and at least one compound chosen from Compound III and pharmaceutically acceptable salts thereof ranges from 3 to 40 percentage points from baseline, i.e., relative to the ppFEV1 of the patient prior to said administration.
291. The method according to embodiment 290, wherein said absolute change in ppFEV1 of said patient ranges from 5 to 30 percentage points.
292. The method according to embodiment 290, wherein said absolute change in ppFEV1 of said patient ranges from 10 to 30 percentage points.
293. The method according to any one of embodiments 285-292, wherein the absolute change in said patient's sweat chloride after 29 days of administration of at least one compound chosen from Compound I and pharmaceutically acceptable salts thereof, at least one compound chosen from Compound II and pharmaceutically acceptable salts thereof, and at least one compound chosen from Compound III and pharmaceutically acceptable salts thereof ranges from −10 to −65 mmol/L from baseline, i.e., relative to the sweat chloride of the patient prior to said administration.
294. The method according to embodiment 293, wherein said absolute change in sweat chloride of said patient ranges from −15 to 65 mmol/L.
295. The method according to any one of embodiments 285-294, wherein the absolute change in said patient's Cystic Fibrosis Questionnaire-Revised (CFQ-R) after 29 days of administration of at least one compound chosen from Compound I and pharmaceutically acceptable salts thereof, at least one compound chosen from Compound II and pharmaceutically acceptable salts thereof, and at least one compound chosen from Compound III and pharmaceutically acceptable salts thereof ranges from −6 to 90 points from baseline, i.e., relative to the CFQ-R of the patient prior to said administration.
296. The method according to embodiment 295, wherein said absolute change in CFQ-R of said patient ranges from 0 to 56 points.
297. The method according to any one of embodiments 285-296, wherein said patient has one F508del mutation and one minimal function mutation and prior to said administration was administered (i) at least one compound chosen from Compound II and pharmaceutically acceptable salts thereof and (ii) at least one compound chosen from Compound III and pharmaceutically acceptable salts thereof, but not at least one compound chosen from Compound I and pharmaceutically acceptable salts thereof.
298. The method according to any one of embodiments 285-296, wherein said patient has two copies of F508del mutation and prior to said administration was administered (i) at least one compound chosen from Compound II and pharmaceutically acceptable salts thereof and (ii) at least one compound chosen from Compound III and pharmaceutically acceptable salts thereof, but not at least one compound chosen from Compound I and pharmaceutically acceptable salts thereof.
299. The method according to any one of claims 285-298, wherein said at least one compound chosen from Compound I and pharmaceutically acceptable salts thereof is comprised in a first pharmaceutical composition; said at least one compound chosen from Compound II and pharmaceutically acceptable salts thereof is comprised in a second pharmaceutical composition; and said at least one compound chosen from Compound III and pharmaceutically acceptable salts thereof is comprised in a third pharmaceutical composition.
300. The method according to any one of claims 285-298, wherein said at least one compound chosen from Compound I and pharmaceutically acceptable salts thereof is comprised in a first pharmaceutical composition; and said at least one compound chosen from Compound II and pharmaceutically acceptable salts thereof and said at least one compound chosen from Compound III and pharmaceutically acceptable salts thereof are comprised in a second pharmaceutical composition.
301. The method according to claim 300, wherein said second pharmaceutical composition comprises one half of the daily dose of said at least one compound chosen from Compound III and pharmaceutically acceptable salts thereof, and the other half of the daily dose of said at least one compound chosen from Compound III and pharmaceutically acceptable salts thereof is administered to said patient in a third pharmaceutical composition.
302. The method according to any one of claims 285-301, wherein said at least one compound chosen from Compound I and pharmaceutically acceptable salts thereof is comprised in a first pharmaceutical composition; said at least one compound chosen from Compound II and pharmaceutically acceptable salts thereof is comprised in a second pharmaceutical composition; and said at least one compound chosen from Compound III and pharmaceutically acceptable salts thereof are comprised in the first pharmaceutical composition.
303. The method according to claim 302, wherein the first pharmaceutical composition is administered to the patient twice daily.
304. The method according to any one of embodiments 285-301, wherein said at least one compound chosen from Compound I and pharmaceutically acceptable salts thereof; said at least one compound chosen from Compound II and pharmaceutically acceptable salts thereof; and said at least one compound chosen from Compound III and pharmaceutically acceptable salts thereof are comprised in a first pharmaceutical composition.
305. The method according to embodiment 304, wherein the first pharmaceutical composition is administered to the patient twice daily.
306. A method of treating cystic fibrosis comprising administering to a patient in need thereof:

(A) 200 mg of Compound I twice daily:

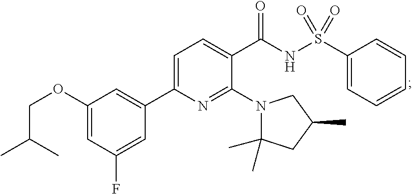 [SMILES: CC(C)COc1cc(F)cc(-c2ccc(C(=O)NS(=O)(=O)c3ccccc3)c(N3C[C@@H](C)CC3(C)C)n2)c1]

(B) 100 mg of Compound II once daily:

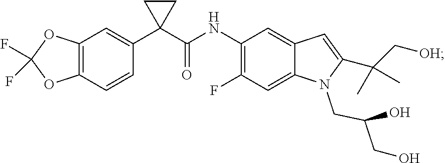 [SMILES: CC(C)(CO)c1cc2cc(NC(=O)C3(c4ccc5c(c4)OC(F)(F)O5)CC3)c(F)cc2n1C[C@@H](O)CO]

and

(C) 150 mg of Compound III twice daily:

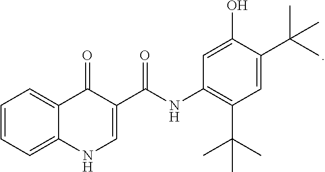 [SMILES: CC(C)(C)c1cc(C(C)(C)C)c(NC(=O)c2c[nH]c3ccccc3c2=O)cc1O]

307. A method of treating cystic fibrosis comprising administering to a patient in need thereof:

(A) 200 mg of Compound I twice daily:

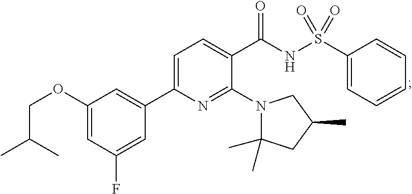 [SMILES: CC(C)COc1cc(F)cc(-c2ccc(C(=O)NS(=O)(=O)c3ccccc3)c(N3C[C@@H](C)CC3(C)C)n2)c1]

(B) 50 mg of Compound II twice daily:

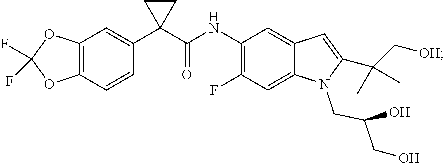 [SMILES: CC(C)(CO)c1cc2cc(NC(=O)C3(c4ccc5c(c4)OC(F)(F)O5)CC3)c(F)cc2n1C[C@@H](O)CO]

and

(C) 150 mg of Compound III twice daily:

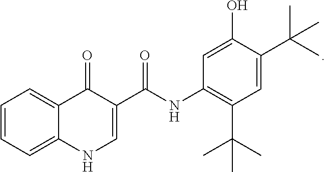 [SMILES: CC(C)(C)c1cc(C(C)(C)C)c(NC(=O)c2c[nH]c3ccccc3c2=O)cc1O]

308. A method of treating cystic fibrosis comprising administering to a patient in need thereof:

(A) 600 mg of Compound I twice daily:

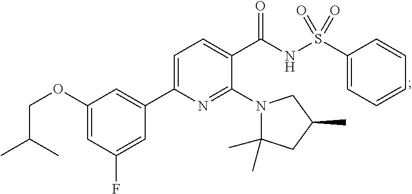 [SMILES: CC(C)COc1cc(F)cc(-c2ccc(C(=O)NS(=O)(=O)c3ccccc3)c(N3C[C@@H](C)CC3(C)C)n2)c1]

(B) 50 mg of Compound II twice daily:

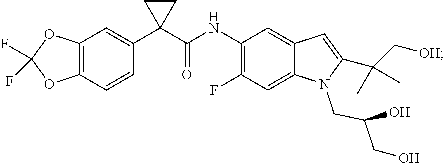 [SMILES: CC(C)(CO)c1cc2cc(NC(=O)C3(c4ccc5c(c4)OC(F)(F)O5)CC3)c(F)cc2n1C[C@@H](O)CO]

and

(C) 300 mg of Compound III twice daily:

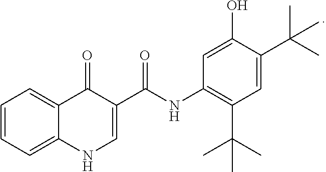 [SMILES: CC(C)(C)c1cc(C(C)(C)C)c(NC(=O)c2c[nH]c3ccccc3c2=O)cc1O]

309. The method according to any one of embodiments 306-308, wherein said patient has cystic fibrosis is chosen from patients with F508del/minimal function genotypes, patients with F508del/F508del genotypes, patients with F508del/ gating genotypes, patients with F508del/residual function genotypes, and patients with F508del/ another CFTR genetic mutation that is expected to be and/or is responsive to the triple combination of Compound I, Compound II, and/or Compound III genotypes based on in vitro and/or clinical data.
310. The method of any one of embodiments 306-308, wherein the patient with a F508del/minimal function genotype has a minimal function mutation selected from:

Mutation

| S4X | C276X | G542X | R792X | E1104X | | |
|---|---|---|---|---|---|---|
| G27X | Q290X | G550X | E822X | R115 | [symbol] | X |

| Q39X | G330X | Q552X | W846X | | | R1162X |
|---|---|---|---|---|---|---|
| W57X | W401X | R553X | Y849X | | | S1196X |
| E60X | Q414X | E585X | R851X | | | W1204X |
| R75X | S434X | G673X | Q890X | | | L1254X |
| E92X | S466X | Q685X | S912X | | | S1255X |
| Q98X | S489X | R709X | Y913X | | | W1282X |
| Y122X | Q493X | K710X | W108 | [symbol] | X | Q1313X |
| E193X | W496X | L732X | Y1092X | | | E1371X |
| L218X | C524X | R764X | W1092X | | | Q1382X |
| Q220X | Q525X | R785X | R1102X | | | Q1411X |
| 185+1G→T | 711+5G→A | 1717−8G→A | 2622+1G→A | | | 3121−1G→A |
| 296+1G→A | 712−1G→A | 1717−1G→A | 2790−1G→C | | | 3500−2A→G |
| 405+1G→A | 1248+1G→A | 1811+1G→C | 3040G→C | | | 3600+2insT |
| 405+3A→C | 1249−1G→A | 1811+1.6kbA→G | (G970 | [symbol] | ) | 3850−1G→A |

| | | | | |
|---|---|---|---|---|
| 406−1G→A | 1341+1G→A | 1812−1G→A | 3120G→A | 4005+1G→A |
| 621+1G→T | 1525−2A→G | 1898+1G→A | 3120+1G→A | 4374+1G→T |
| 711+1G→T | 1525−1G→A | 1898+1G→C | 3121−2A→G | |
| 182delT | 1119delA | 1782delA | 2732insA | 3876delA |
| 306insA | 1138insG | 1824delA | 2869insG | 3878delG |
| 365-366insT | 1154insTC | 2043delG | 2896insAG | 3905insT |
| 394delTT | 1161delC | 2143delT | 2942insT | 4016insT |
| 442delA | 1213delT | 2183AA→G [symbol] | 2957delT | 4021dupT |
| 444delA | 1259insA | 2184delA | 3007delG | 4040delA |
| 457TAT→G | 1288insTA | 2184insA | 3028delA | 4279insA |
| 541delC | 1471delA | 2307insA | 3171delC | 4326delTC |
| 574delA | 1497delGG | 2347delG | 3659delC | |
| 663delT | 1548delG | 2585delT | 3737delA | |
| 935delA | 1609del CA | 2594delGT | 3791delC | |
| 1078delT | 1677delTA | 2711delT | 3821delT | |

| | | |
|---|---|---|
| CFTRdele2,3 | 1461ins4 | 2991del32 |
| CFTRdele22,23 | 1924del7 | 3199del6 [symbol] |
| 124del23bp | 2055del9→A | 3667ins4 |
| 852del22 | 2105-2117del13insAGAAA | 4010del4 |
| 991del5 | 2721del11 | 4209TGTT→AA |

| | | | |
|---|---|---|---|
| A46D [symbol] | V520F | Y569D [symbol] | N1303K |
| G85E | A559T [symbol] | L1065P | |
| R347P | R560T | R1066C | |
| L467P [symbol] | R560S | L1077P [symbol] | |
| I507del | A561E | M1101K | |

[symbol]

indicates data missing or illegible when filed

311. The method according to any one of embodiments 306-310, wherein the absolute change in said patient's percent predicted forced expiratory volume in one second (ppFEV₁) after 29 days of administration of Compound I, Compound II, and Compound III ranges from 3 to 40 percentage points from baseline, i.e., relative to the ppFEV1 of the patient prior to said administration.
312. The method according to embodiment 311, wherein said absolute change in ppFEV1 of said patient ranges from 5 to 30 percentage points.
313. The method according to embodiment 311, wherein said absolute change in ppFEV1 of said patient ranges from 10 to 30 percentage points.
314. The method according to any one of embodiments 306-313, wherein the absolute change in said patient's sweat chloride after 29 days of administration of Compound I, Compound II, and Compound III ranges from −10 to −65 mmol/L from baseline, i.e., relative to the sweat chloride of the patient prior to said administration.
315. The method according to embodiment 314, wherein said absolute change in sweat chloride of said patient ranges from −15 to 65 mmol/L.
316. The method according to any one of embodiments 306-315, wherein the absolute change in said patient's Cystic Fibrosis Questionnaire-Revised (CFQ-R) after 29 days of administration of Compound I, Compound II, and Compound III ranges from −6 to 90 points from baseline, i.e., relative to the CFQ-R of the patient prior to said administration.
317. The method according to embodiment 316, wherein said absolute change in CFQ-R of said patient ranges from 0 to 56 points.
318. The method according to any one of embodiments 306-317, wherein said patient has one F508del mutation and one minimal function mutation and prior to said administration was administered Compound II and Compound III, but not Compound I.
319. The method according to any one of embodiments 306-317, wherein said patient has two copies of F508del mutation and prior to said administration was administered Compound II and Compound III, but not Compound I.

320. The method according to any one of embodiments 306-319, wherein Compound I is comprised in a first pharmaceutical composition; Compound II is comprised in a second pharmaceutical composition; and Compound III is comprised in a third pharmaceutical composition.

321. The method according to any one of embodiments 306-319, wherein Compound I is comprised in a first pharmaceutical composition; Compound II and Compound III are comprised in a second pharmaceutical composition.

322. The method according to embodiment 306-319, wherein said second pharmaceutical composition comprises one half of the daily dose of Compound III, and the other half of the daily dose of Compound III is administered to said patient in a third pharmaceutical composition.
323. The method according to any one of embodiments 306-319, wherein said Compound I is comprised in a first pharmaceutical composition; Compound II is comprised in a second pharmaceutical composition; and Compound III is comprised in the first pharmaceutical composition.
324. The method according to embodiment 323, wherein the first pharmaceutical composition is administered to the patient twice daily.
325. The method according to any one of embodiments 306-319, wherein Compound I; Compound II; and Compound III are comprised in a first pharmaceutical composition.
326. The method according to embodiment 325, wherein the first pharmaceutical composition is administered to the patient twice daily.

### Methods of Preparing Compounds

### General Experimental Procedures

Reagents and starting materials were obtained by commercial sources unless otherwise stated and were used without purification. Proton and carbon NMR spectra were acquired on either of a Bruker Biospin DRX 400 MHz FTNMR spectrometer operating at a ¹Hand ¹³C resonant frequency of 400 and 100 MHz respectively, or on a 300 MHz NMR spectrometer. One dimensional proton and carbon spectra were acquired using a broadband observe (BBFO) probe with 20 Hz sample rotation at 0.1834 and 0.9083 Hz/Pt digital resolution respectively. All proton and carbon spectra were acquired with temperature control at 30° C. using standard, previously published pulse sequences and routine processing parameters. Final purity of compounds was determined by reversed phase UPLC using an Acquity UPLC BEH C18 column (50×2.1 mm, 1.7 μm particle) made by Waters (pn: 186002350), and a dual gradient run from 1-99% mobile phase B over 3.0 minutes. Mobile phase A=H₂O (0.05% CF₃CO₂H). Mobile phase B=CH₃CN (0.035% CF₃CO₂H). Flow rate=1.2 mL/min, injection volume=1.5 μL, and column temperature=60° C. Final purity was calculated by averaging the area under the curve (AUC) of two UV traces (220 nm, 254 nm). Low-resolution mass spectra were obtained using a single quadrupole mass spectrometer with a mass accuracy of 0.1 Da and a minimum resolution of 1000 amu across the detection range using electrospray ionization (ESI) using the hydrogen ion (H⁺). Optical purity of methyl (2S)-2,4-dimethyl-4-nitro-pentanoate was determined using chiral gas chromatography (GC) analysis on an Agilent 7890A/MSD 5975C instrument, using a Restek Rt-βDEXcst (30m×0.25 mm×0.25 um_df) column, with a 2.0 mL/min flow rate (H2 carrier gas), at an injection temperature of 220° C. and an oven temperature of 120° C., 15 minutes.

### EXAMPLES

Compounds I, II and III can be prepared by any suitable method in the art, for example, PCT Publication Nos. WO 2011/133751 and WO 2015/160787.

### Example 1: Synthesis of Compound I

### Part A: Synthesis of (4S)-2,2,4-trimethylpyrrolidine hydrochloride

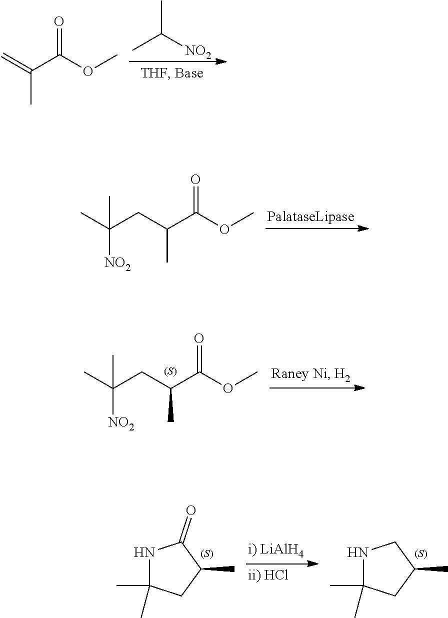

### Step 1: Synthesis of methyl-2,4-dimethyl-4-nitro-pentanoate

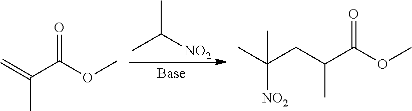

Tetrahydrofuran (THF, 4.5 L) was added to a 20 L glass reactor and stirred under N2 at room temperature. 2-Nitropropane (1.5 kg, 16.83 mol) and 1,8-Diazabicyclo[5.4.0]undec-7-ene (DBU) (1.282 kg, 8.42 mol) were then charged to the reactor, and the jacket temperature was increased to 50° C. Once the reactor contents were close to 50° C., methyl methacrylate (1.854 kg, 18.52 mol) was added slowly over 100 minutes. The reaction temperature was maintained at or close to 50° C. for 21 hours. The reaction mixture was concentrated in vacuo then transferred back to the reactor and diluted with methyl tert-butyl ether (MTBE) (14 L). 2 M HCl (7.5 L) was added, and this mixture was stirred for 5 minutes then allowed to settle. Two clear layers were visible—a lower yellow aqueous phase and an upper green organic phase. The aqueous layer was removed, and the organic layer was stirred again with 2 M HCl (3 L). After separation, the HCl washes were recombined and stirred with MTBE (3 L) for 5 minutes. The aqueous layer was removed, and all of the organic layers were combined in the reactor and stirred with water (3 L) for 5 minutes. After separation, the organic layers were concentrated in vacuo to afford a cloudy green oil. Crude product was treated with MgSO₄ and filtered to afford methyl-2,4-dimethyl-4-nitro-pentanoate as a clear green oil (3.16 kg, 99% yield). ¹H NMR (400 MHz, Chloroform-d) δ 3.68 (s, 3H), 2.56-2.35 (m, 2H), 2.11-2.00 (m, 1H), 1.57 (s, 3H), 1.55 (s, 3H), 1.19 (d, J=6.8 Hz, 3H).

### Step 2: Synthesis of methyl (2S)-2,4-dimethyl-4-nitro-pentanoate

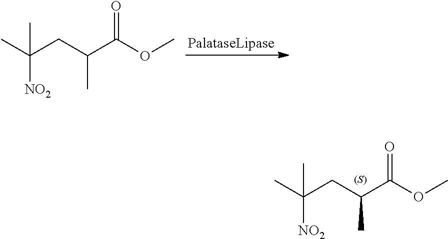

A reactor was charged with purified water (2090 L; 10 vol) and then potassium phosphate monobasic (27 kg, 198.4 moles; 13 g/L for water charge). The pH of the reactor contents was adjusted to pH 6.5 (±0.2) with 20% (w/v) potassium carbonate solution. The reactor was charged with racemic methyl-2,4-dimethyl-4-nitro-pentanoate (209 kg; 1104.6 moles), and Palatase 20000 L lipase (13 L, 15.8 kg; 0.06 vol).

The reaction mixture was adjusted to 32±2° C. and stirred for 15-21 hours, and pH 6.5 was maintained using a pH stat with the automatic addition of 20% potassium carbonate solution. When the racemic starting material was converted to >98% ee of the S-enantiomer, as determined by chiral GC, external heating was switched off The reactor was then charged with MTBE (35 L; 5 vol), and the aqueous layer was extracted with MTBE (3 times, 400-1000 L). The combined organic extracts were washed with aqueous Na₂CO₃ (4 times, 522 L, 18% w/w 2.5 vol), water (523 L; 2.5 vol), and 10% aqueous NaCl (314 L, 1.5 vol). The organic layer was concentrated in vacuo to afford methyl (2S)-2,4-dimethyl-4-nitro-pentanoate as a mobile yellow oil (>98% ee, 94.4 kg; 45% yield).

### Step 3: Synthesis of (3S)-3,5,5-trimethylpyrrolidin-2-one

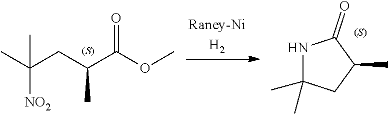

A 20 L reactor was purged with N2. The vessel was charged sequentially with DI water-rinsed, damp Raney® Ni (2800 grade, 250 g), methyl (2S)-2,4-dimethyl-4-nitro-pentanoate (1741 g, 9.2 mol), and ethanol (13.9 L, 8 vol). The reaction was stirred at 900 rpm, and the reactor was flushed with H2 and maintained at ˜2.5 bar. The reaction mixture was then warmed to 60° C. for 5 hours. The reaction mixture was cooled and filtered to remove Raney nickel, and the solid cake was rinsed with ethanol (3.5 L, 2 vol). The ethanolic solution of the product was combined with a second equal sized batch and concentrated in vacuo to reduce to a minimum volume of ethanol (˜1.5 volumes). Heptane (2.5 L) was added, and the suspension was concentrated again to ˜1.5 volumes. This was repeated 3 times; the resulting suspension was cooled to 0-5° C., filtered under suction, and washed with heptane (2.5 L). The product was dried under vacuum for 20 minutes then transferred to drying trays and dried in a vacuum oven at 40° C. overnight to afford (3S)-3,5,5-trimethylpyrrolidin-2-one as a white crystalline solid (2.042 kg, 16.1 mol, 87%). ¹H NMR (400 MHz, Chloroform-d) δ 6.39 (s, 1H), 2.62 (ddq, J=9.9, 8.6, 7.1 Hz, 1H), 2.17 (dd, J=12.4, 8.6 Hz, 1H), 1.56 (dd, J=12.5, 9.9 Hz, 1H), 1.31 (s, 3H), 1.25 (s, 3H), 1.20 (d, J=7.1 Hz, 3H).

### Step 4: Synthesis of (4S)-2,2,4-trimethylpyrrolidine hydrochloride

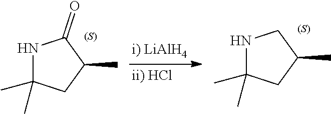

A glass lined 120 L reactor was charged with lithium aluminium hydride pellets (2.5 kg, 66 mol) and dry THF (60 L) and warmed to 30° C. The resulting suspension was charged with (S)-3,5,5-trimethylpyrrolidin-2-one (7.0 kg, 54 mol) in THF (25 L) over 2 hours while maintaining the reaction temperature at 30 to 40° C. After complete addition, the reaction temperature was increased to 60-63° C. and maintained overnight. The reaction mixture was cooled to 22° C., then cautiously quenched with the addition of ethyl acetate (EtOAc) (1.0 L, 10 moles), followed by a mixture of THF (3.4 L) and water (2.5 kg, 2.0 eq), and then a mixture of water (1.75 kg) with 50% aqueous sodium hydroxide (750 g, 2 equiv water with 1.4 equiv sodium hydroxide relative to aluminum), followed by 7.5 L water. After the addition was complete, the reaction mixture was cooled to room temperature, and the solid was removed by filtration and washed with THF (3×25 L). The filtrate and washings were combined and treated with 5.0 L (58 moles) of aqueous 37% HCl (1.05 equiv.) while maintaining the temperature below 30° C. The resultant solution was concentrated by vacuum distillation to a slurry. Isopropanol (8 L) was added and the solution was concentrated to near dryness by vacuum distillation. Isopropanol (4 L) was added, and the product was slurried by warming to 50° C. MTBE (6 L) was added, and the slurry was cooled to 2-5° C. The product was collected by filtration and rinsed with 12 L MTBE and dried in a vacuum oven (55° C./300 torr/N2 bleed) to afford (4S)-2,2,4-trimethylpyrrolidine.HCl as a white, crystalline solid (6.21 kg, 75% yield). ¹H NMR (400 MHz, DMSO-d6) δ 9.34 (br d, 2H), 3.33 (dd, J=11.4, 8.4 Hz, 1H), 2.75 (dd, J=11.4, 8.6 Hz, 1H), 2.50-2.39 (m, 1H), 1.97 (dd, J=12.7, 7.7 Hz, 1H), 1.42 (s, 3H), 1.38 (dd, J=12.8, 10.1 Hz, 1H), 1.31 (s, 3H), 1.05 (d, J=6.6 Hz, 3H).

### Part B: Synthesis of N-(benzenesulfonyl)-6-(3-fluoro-5-isobutoxy-phenyl)-2-[(4S)-2,2,4-trimethylpyrrolidin-1-yl]pyridine-3-carboxamide (Compound I)

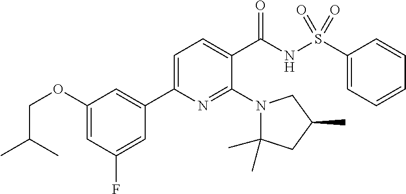 [SMILES: CC(C)COc1cc(F)cc(-c2ccc(C(=O)NS(=O)(=O)c3ccccc3)c(N3C[C@@H](C)CC3(C)C)n2)c1]

### Step 1: Synthesis of tert-Butyl 2,6-dichloropyridine-3-carboxylate

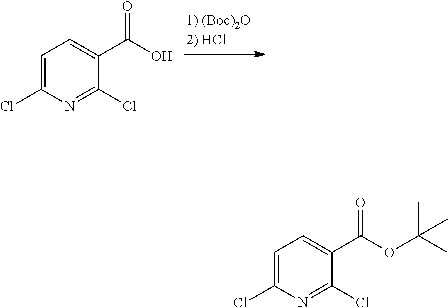

A solution of 2,6-dichloropyridine-3-carboxylic acid (10 g, 52.08 mmol) in THF (210 mL) was treated successively with di-tert-butyl dicarbonate (17 g, 77.89 mmol) and 4-(dimethylamino)pyridine (3.2 g, 26.19 mmol) and stirred overnight at room temperature. At this point, HCl 1N (400 mL) was added, and the mixture was stirred vigorously for 10 minutes. The product was extracted with ethyl acetate (2×300 mL), and the combined organic layers were washed with water (300 mL) and brine (150 mL) and dried over sodium sulfate and concentrated under reduced pressure to give 12.94 g (96% yield) of tert-butyl 2,6-dichloropyridine-3-carboxylate as a colorless oil. ESI-MS m/z calc. 247.02, found 248.1 (M+1)⁺; Retention time: 2.27 minutes. ¹H NMR (300 MHz, CDCl₃) ppm 1.60 (s, 9H), 7.30 (d, J=7.9 Hz, 1H), 8.05 (d, J=8.2 Hz, 1H).

### Step 2: tert-butyl 2-chloro-6-(3-fluoro-5-isobutoxy-phenyl)pyridine-3-carboxylate

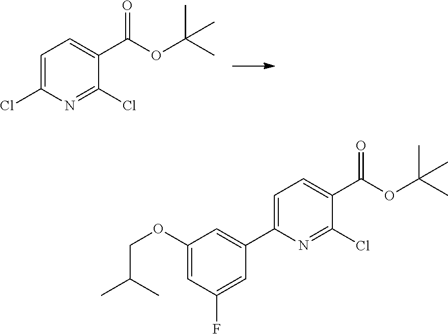 [SMILES: CC(C)(C)OC(=O)c1ccc(Cl)nc1Cl ; CC(C)COc1cc(F)cc(-c2ccc(C(=O)OC(C)(C)C)c(Cl)n2)c1]

tert-Butyl 2,6-dichloropyridine-3-carboxylate (15.0 g, 60.5 mmol) and (3-fluoro-5-isobutoxy-phenyl)boronic acid (13.46 g, 63.48 mmol) were combined and fully dissolved in ethanol (150 mL) and toluene (150 mL). A suspension of sodium carbonate (19.23 g, 181.4 mmol) in water (30 mL) was added. Tetrakis(triphenylphosphine)palladium (0) (2.096 g, 1.814 mmol) was added under nitrogen. The reaction mixture was allowed to stir at 60° C. for 16 hours. Volatiles were removed under reduced pressure. The remaining solids were partitioned between water (100 mL) and ethyl acetate (100 mL). The organic layer was washed with brine (lx 100 mL), dried over sodium sulfate, filtered and concentrated under reduced pressure. The material was subjected silica gel column chromatography on a 330 gram silica gel column, 0 to 20% ethyl acetate in hexanes gradient. The material was repurified on a 220 gram silica gel column, isocratic 100% hexane for 10 minutes, then a 0 to 5% ethyl acetate in hexanes gradient to yield tert-butyl 2-chloro-6-(3-fluoro-5-isobutoxy-phenyl)pyridine-3-carboxylate (18.87 g, 49.68 mmol, 82.2%) was obtained as a colorless oil. ¹H NMR (400 MHz, DMSO-d6) δ 8.24 (d, J=8.0 Hz, 1H), 8.16 (d, J=8.1 Hz, 1H), 7.48 (dd, J=9.4, 2.0 Hz, 2H), 6.99 (dt, J=10.8, 2.2 Hz, 1H), 3.86 (d, J=6.5 Hz, 2H), 2.05 (dt, J=13.3, 6.6 Hz, 1H), 1.57 (d, J=9.3 Hz, 9H), 1.00 (t, J=5.5 Hz, 6H). ESI-MS m/z calc. 379.13504, found 380.2 (M+1)⁺; Retention time: 2.57 minutes.

### Step 3: 2-chloro-6-(3-fluoro-5-isobutoxy-phenyl)pyridine-3-carboxylic acid

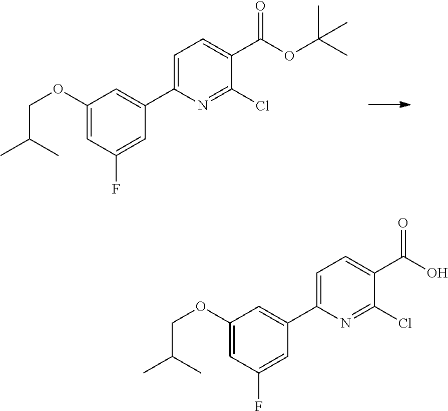

tert-Butyl 2-chloro-6-(3-fluoro-5-isobutoxy-phenyl)pyridine-3-carboxylate (18.57 g, 48.89 mmol) was dissolved in dichloromethane (200 mL). Trifluoroacetic acid (60 mL, 780 mmol) was added and the reaction mixture was allowed to stir at room temperature for 1 hour. The reaction mixture was stirred at 40° C. for 2 hours. The reaction mixture was concentrated under reduced pressure and taken up in ethyl acetate (100 mL). It was washed with a saturated aqueous sodium bicarbonate solution (lx 100 mL) and brine (1×100 mL), dried over sodium sulfate, filtered and concentrated under reduced pressure. The crude product was suspended in ethyl acetate (75 mL) and washed with aqueous HCl (1 N, 1×75 mL). The organic layer was dried over sodium sulfate, filtered and concentrated under reduced pressure. The remaining solid (17.7 g) was stirred as a slurry in dichloromethane (35 mL) at 40° C. for 30 minutes. After cooling to room temperature, the remaining slurry was filtered, and then rinsed with cold dichloromethane to give 2-chloro-6-(3-fluoro-5-isobutoxy-phenyl)pyridine-3-carboxylic acid (11.35 g, 35.06 mmol, 72%) as a white solid. ¹H NMR (400 MHz, DMSO-d6) δ 13.76 (s, 1H), 8.31 (d, J=8.0 Hz, 1H), 8.17 (d, J=8.1 Hz, 1H), 7.54-7.47 (m, 2H), 7.00 (dt, J=10.8, 2.3 Hz, 1H), 3.87 (d, J=6.5 Hz, 2H), 2.05 (dt, J=13.3, 6.6 Hz, 1H), 1.01 (d, J=6.7 Hz, 6H). ESI-MS m/z calc. 323.1, found 324.1 (M+1)⁺; Retention time: 1.96 minutes.

### Step 4: N-(benzenesulfonyl)-2-chloro-6-(3-fluoro-5-isobutoxy-phenyl)pyridine-3-carboxamide

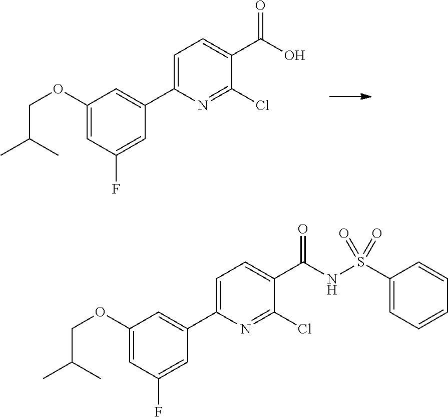 [SMILES: CC(C)COc1cc(F)cc(-c2ccc(C(=O)O)c(Cl)n2)c1 ; CC(C)COc1cc(F)cc(-c2ccc(C(=O)NS(=O)(=O)c3ccccc3)c(Cl)n2)c1]

To a solution of the 2-chloro-6-(3-fluoro-5-isobutoxy-phenyl)pyridine-3-carboxylic acid (10.0 g, 30.89 mmol) in DMF (30.0 mL) at ambient temperature in a round bottom flask was slowly added carbonyldiimidazole (5.510 g, 33.98 mmol) portionwise and the mixture stirred for 100 min. Meanwhile to benzenesulfonamide (6.069 g, 38.61 mmol) in DMF (30.0 mL) (homogenous solution) in another round bottom flask was added NaHMDS in THF (38.61 mL of 1.0 M, 38.61 mmol) portionwise via syringe over 30-45 min and on completion of addition the mixture was stirred a further 30 min. The mixture containing the activated acid was then added to the mixture containing the deprotonated sulfonamide and the combined mixture was stirred 1 h. The reaction was cooled with a 0° C. bath and quenched by addition of 12N aqueous HCl (11.58 mL) in portions over 2-3 minutes resulting in precipitated solids. Transferred the reaction mixture to a separatory funnel and ethyl acetate (100.0 mL) was added. Added 1N aqueous HCl (20.0 mL) giving a pH=2-3 then separated the layers and washed the organic layer with 5:1 water/saturated aqueous brine (120.0 mL), saturated aqueous brine (1×50 mL, 1×30 mL), dried (sodium sulfate), filtered and concentrated under reduced pressure to a clear light yellow oil that was concentrated from isopropanol several more times resulting in precipitation of a solid. The solid was slurried overnight in isopropanol then filtered and washed the solid with heptane (50 mL) and dried in vacuo giving N-(benzenesulfonyl)-2-chloro-6-(3-fluoro-5-isobutoxy-phenyl)pyridine-3-carboxamide (10.22 g, 22.08 mmol, 71.47%) as a white solid. ¹H NMR (400 MHz, DMSO) δ 12.85 (s, 1H), 8.15 (d, J=8.0 Hz, 1H), 8.09 (d, J=8.0 Hz, 1H), 8.02 (dd, J=5.3, 3.3 Hz, 2H), 7.76 (d, J=7.4 Hz, 1H), 7.69 (t, J=7.6 Hz, 2H), 7.51-7.43 (m, 2H), 6.99 (dd, J=10.8, 2.3 Hz, 1H), 3.85 (d, J=6.5 Hz, 2H), 2.04 (dt, J=13.3, 6.6 Hz, 1H), 1.00 (d, J=6.7 Hz, 6H). ESI-MS m/z calc. 462.08163, found 463.19 (M+1)⁺; Retention time: 2.93 minutes [5 minute method].

### Step 5: N-(benzenesulfonyl)-6-(3-fluoro-5-isobutoxy-phenyl)-2-[(4S)-2,2,4-trimethylpyrrolidin-1-yl]pyridine-3-carboxamide

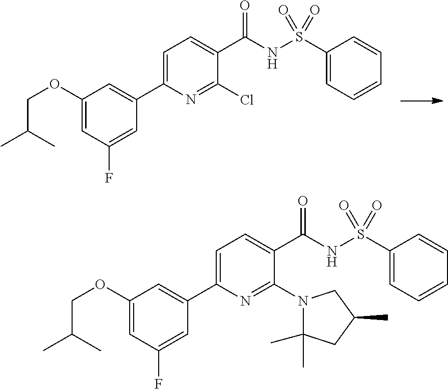 [SMILES: CC(C)COc1cc(F)cc(-c2ccc(C(=O)NS(=O)(=O)c3ccccc3)c(Cl)n2)c1 ; CC(C)COc1cc(F)cc(-c2ccc(C(=O)NS(=O)(=O)c3ccccc3)c(N3C[C@@H](C)CC3(C)C)n2)c1]

To a round bottom flask outfitted with a reflux condenser was added N-(benzenesulfonyl)-2-chloro-6-(3-fluoro-5-isobutoxy-phenyl)pyridine-3-carboxamide (10.0 g, 21.60 mmol) and NMP (40 mL) and stirring was commenced. Warmed to 50° C. and began portionwise addition of potassium carbonate (5.970 g, 43.20 mmol) followed by (4S)-2,2,4-trimethylpyrrolidine (4.890 g, 43.20 mmol) in one portion. After stirring for 10 min, heated the mixture to 125° C. for 65 h, then cooled to 10° C. and added 1N aqueous HCl (50.0 mL, 50.00 mmol) in portions to give pH 1-2 and a precipitated solid. Added ethyl acetate (100.0 mL) to dissolve solid and diluted the aqueous layer with water (50.0 mL) and stirred for 10 min. The mixture was transferred to a separatory funnel and layers were allowed to separate. Added aqueous 1N HCl dropwise until all solids were dissolved. Separated the layers and the aqueous layer was back extracted with ethyl acetate (50.00 mL) followed by combination of the organic layers. To the combined organic layers was added water (50.00 mL) giving an emulsion which was clarified by the addition of 1N aqueous HCl (25.00 mL). Separated the layers then the organic layer was washed with saturated aqueous brine (50.00 mL), dried over Na₂SO₄, filtered through celite and rinsed with ethyl acetate (30.00 mL). The filtrate was concentrated under reduced pressure and the residue was purified by silica gel chromatography using a gradient from 100% hexanes to 50% EtOAc giving a light amber oil which was evaporated from isopropanol several times under reduced pressure providing N-(benzenesulfonyl)-6-(3-fluoro-5-isobutoxy-phenyl)-2-[(4S)-2,2,4-trimethylpyrrolidin-1-yl]pyridine-3-carboxamide (9.73 g, 18.03 mmol, 83.5%) as a yellow solid. ¹H NMR (400 MHz, DMSO-d6) δ 12.57 (s, 1H), 8.16-7.88 (m, 2H), 7.82-7.57 (m, 4H), 7.47 (t, J=1.8 Hz, 1H), 7.40 (dt, J=9.9, 2.0 Hz, 1H), 7.27 (d, J=8.1 Hz, 1H), 6.89 (dt, J=10.8, 2.3 Hz, 1H), 3.83 (d, J=6.6 Hz, 2H), 2.48-2.28 (m, 2H), 2.07 (dtt, J=26.6, 13.4, 6.4 Hz, 2H), 1.83 (dd, J=11.9, 5.5 Hz, 1H), 1.57 (d, J=17.3 Hz, 6H), 1.38 (t, J=12.1 Hz, 1H), 1.04 (d, J=6.1 Hz, 1H), 0.98 (d, J=6.7 Hz, 6H), 0.66 (d, J=6.3 Hz, 3H). ESI-MS m/z calc. 539.2254, found 540.0 (M+1)⁺; Retention time: 3.25 minutes [5 minute method].

### Example 2

### Synthesis of Compound II: (R)-1-(2,2-Difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropanecarboxamide

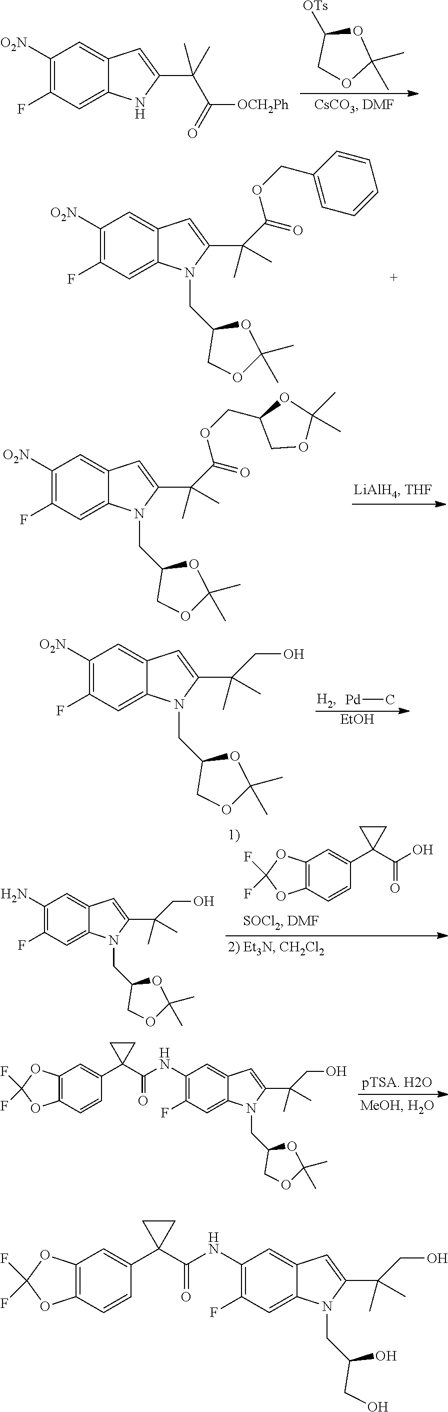

### Step 1: (R)-Benzyl 2-(1-((2,2-dimethyl-1,3-dioxolan-4-yl)methyl)-6-fluoro-5-nitro-1H-indol-2-yl)-2-methylpropanoate and ((S)-2,2-Dimethyl-1,3-dioxolan-4-yl)methyl 2-(1-(((R)-2,2-dimethyl-1,3-dioxolan-4-yl)methyl)-6-fluoro-5-nitro-1H-indol-2-yl)-2-methylpropanoate

Cesium carbonate (8.23 g, 25.3 mmol) was added to a mixture of benzyl 2-(6-fluoro-5-nitro-1H-indol-2-yl)-2-methylpropanoate (3.0 g, 8.4 mmol) and (S)-(2,2-dimethyl-1,3-dioxolan-4-yl)methyl 4-methylbenzenesulfonate (7.23 g, 25.3 mmol) in DMF (17 mL). The reaction was stirred at 80° C. for 46 hours under a nitrogen atmosphere. The mixture was then partitioned between ethyl acetate and water. The aqueous layer was extracted with ethyl acetate. The combined ethyl acetate layers were washed with brine, dried over MgSO₄, filtered and concentrated. The crude product, a viscous brown oil which contains both of the products shown above, was taken directly to the next step without further purification. (R)-Benzyl 2-(1-((2,2-dimethyl-1,3-dioxolan-4-yl)methyl)-6-fluoro-5-nitro-1H-indol-2-yl)-2-methylpropanoate, ESI-MS m/z calc. 470.2, found 471.5 (M+1)⁺. Retention time 2.20 minutes. ((S)-2,2-Dimethyl-1,3-dioxolan-4-yl)methyl 2-(1-(((R)-2,2-dimethyl-1,3-dioxolan-4-yl)methyl)-6-fluoro-5-nitro-1H-indol-2-yl)-2-methylpropanoate, ESI-MS m/z calc. 494.5, found 495.7 (M+1)⁺. Retention time 2.01 minutes.

### Step 2: (R)-2-(1-((2,2-dimethyl-1,3-dioxolan-4-yl)methyl)-6-fluoro-5-nitro-1H-indol-2-yl)-2-methylpropan-1-ol

The crude reaction mixture obtained in step (A) was dissolved in THF (42 mL) and cooled in an ice-water bath. LiAlH₄ (16.8 mL of 1 M solution, 16.8 mmol) was added drop-wise. After the addition was complete, the mixture was stirred for an additional 5 minutes. The reaction was quenched by adding water (1 mL), 15% NaOH solution (1 mL) and then water (3 mL). The mixture was filtered over Celite, and the solids were washed with THF and ethyl acetate. The filtrate was concentrated and purified by column chromatography (30-60% ethyl acetate-hexanes) to obtain (R)-2-(1-((2,2-dimethyl-1,3-dioxolan-4-yl)methyl)-6-fluoro-5-nitro-1H-indol-2-yl)-2-methylpropan-1-ol as a brown oil (2.68 g, 87% over 2 steps). ESI-MS m/z calc. 366.4, found 367.3 (M+1)⁺. Retention time 1.68 minutes. ¹H NMR (400 MHz, DMSO-d6) δ 8.34 (d, J=7.6 Hz, 1H), 7.65 (d, J=13.4 Hz, 1H), 6.57 (s, 1H), 4.94 (t, J=5.4 Hz, 1H), 4.64-4.60 (m, 1H), 4.52-4.42 (m, 2H), 4.16-4.14 (m, 1H), 3.76-3.74 (m, 1H), 3.63-3.53 (m, 2H), 1.42 (s, 3H), 1.38-1.36 (m, 6H) and 1.19 (s, 3H) ppm

### Step 3: (R)-2-(5-amino-1-((2,2-dimethyl-1,3-dioxolan-4-yl)methyl)-6-fluoro-1H-indol-2-yl)-2-methylpropan-1-ol

(R)-2-(1-((2,2-dimethyl-1,3-dioxolan-4-yl)methyl)-6-fluoro-5-nitro-1H-indol-2-yl)-2-methylpropan-1-ol (2.5 g, 6.82 mmol) was dissolved in ethanol (70 mL) and the reaction was flushed with N2. Then Pd—C (250 mg, 5% wt) was added. The reaction was flushed with nitrogen again and then stirred under H2 (atm). After 2.5 hours only partial conversion to the product was observed by LCMS. The reaction was filtered through Celite and concentrated. The residue was re-subjected to the conditions above. After 2 hours LCMS indicated complete conversion to product. The reaction mixture was filtered through Celite. The filtrate was concentrated to yield the product as a black solid (1.82 g, 79%). ESI-MS m/z calc. 336.2, found 337.5 (M+1)⁺. Retention time 0.86 minutes. ¹H NMR (400 MHz, DMSO-d6) δ 7.17 (d, J=12.6 Hz, 1H), 6.76 (d, J=9.0 Hz, 1H), 6.03 (s, 1H), 4.79-4.76 (m, 1H), 4.46 (s, 2H), 4.37-4.31 (m, 3H), 4.06 (dd, J=6.1, 8.3 Hz, 1H), 3.70-3.67 (m, 1H), 3.55-3.52 (m, 2H), 1.41 (s, 3H), 1.32 (s, 6H) and 1.21 (s, 3H) ppm.

### Step 4: (R)-1-(2,2-difluorobenzo[d][1,3]dioxol-5-yl)-N-(1-((2,2-dimethyl-1,3-dioxolan-4-yl)methyl)-6-fluoro-2-(1-hydroxy-2-methylpropan-2-yl)-1H-indol-5-yl)cyclopropanecarboxamide

DMF (3 drops) was added to a stirring mixture of 1-(2,2-difluorobenzo[d][1,3]dioxol-5-yl)cyclopropanecarboxylic acid (1.87 g, 7.7 mmol) and thionyl chloride (1.30 mL, 17.9 mmol). After 1 hour a clear solution had formed. The solution was concentrated under vacuum and then toluene (3 mL) was added and the mixture was concentrated again. The toluene step was repeated once more and the residue was placed on high vacuum for 10 minutes. The acid chloride was then dissolved in dichloromethane (10 mL) and added to a mixture of (R)-2-(5-amino-1-((2,2-dimethyl-1,3-dioxolan-4-yl)methyl)-6-fluoro-1H-indol-2-yl)-2-methylpropan-1-ol (1.8 g, 5.4 mmol) and triethylamine (2.24 mL, 16.1 mmol) in dichloromethane (45 mL). The reaction was stirred at room temperature for 1 hour. The reaction was washed with 1N HCl solution, saturated NaHCO₃solution and brine, dried over MgSO₄ and concentrated to yield the product as a black foamy solid (3 g, 100%). ESI-MS m/z calc. 560.6, found 561.7 (M+1)⁺. Retention time 2.05 minutes. ¹H NMR (400 MHz, DMSO-d6) δ 8.31 (s, 1H), 7.53 (s, 1H), 7.42-7.40 (m, 2H), 7.34-7.30 (m, 3H), 6.24 (s, 1H), 4.51-4.48 (m, 1H), 4.39-4.34 (m, 2H), 4.08 (dd, J=6.0, 8.3 Hz, 1H), 3.69 (t, J=7.6 Hz, 1H), 3.58-3.51 (m, 2H), 1.48-1.45 (m, 2H), 1.39 (s, 3H), 1.34-1.33 (m, 6H), 1.18 (s, 3H) and 1.14-1.12 (m, 2H) ppm

### Step 5: (R)-1-(2,2-difluorobenzo [d][1,3]dioxol-5-yl)-N-(1-(2,3-dihydroxypropyl)-6-fluoro-2-(1-hydroxy-2-methylpropan-2-yl)-1H-indol-5-cyclopropanecarboxamide

(R)-1-(2,2-difluorobenzo[d][1,3]dioxol-5-yl)-N-(1-((2,2-dimethyl-1,3-dioxolan-4-yl)methyl)-6-fluoro-2-(1-hydroxy-2-methylpropan-2-yl)-1H-indol-5-yl)cyclopropanecarboxamide (3.0 g, 5.4 mmol) was dissolved in methanol (52 mL). Water (5.2 mL) was added followed by p-TsOH.H₂O (204 mg, 1.1 mmol). The reaction was heated at 80° C. for 45 minutes. The solution was concentrated and then partitioned between ethyl acetate and saturated NaHCO₃solution. The ethyl acetate layer was dried over MgSO₄ and concentrated. The residue was purified by column chromatography (50-100% ethyl acetate-hexanes) to yield the product as a cream colored foamy solid. (1.3 g, 47%, ee>98% by SFC). ESI-MS m/z calc. 520.5, found 521.7 (M+1)⁺. Retention time 1.69 minutes. ¹H NMR (400 MHz, DMSO-d6) δ 8.31 (s, 1H), 7.53 (s, 1H), 7.42-7.38 (m, 2H), 7.33-7.30 (m, 2H), 6.22 (s, 1H), 5.01 (d, J=5.2 Hz, 1H), 4.90 (t, J=5.5 Hz, 1H), 4.75 (t, J=5.8 Hz, 1H), 4.40 (dd, J=2.6, 15.1 Hz, 1H), 4.10 (dd, J=8.7, 15.1 Hz, 1H), 3.90 (s, 1H), 3.65-3.54 (m, 2H), 3.48-3.33 (m, 2H), 1.48-1.45 (m, 2H), 1.35 (s, 3H), 1.32 (s, 3H) and 1.14-1.11 (m, 2H) ppm.

### Example 3

### Synthesis of Compound III: N-(2,4-di-tert-butyl-5-hydroxyphenyl)-4-oxo-1,4-dihydroquinoline-3-carboxamide

### Part A: Synthesis of 4-oxo-1,4-dihydroquinoline-3-carboxylic acid

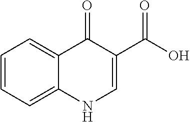 [SMILES: O=C(O)c1c[nH]c2ccccc2c1=O]

### Step 1: 2-Phenylaminomethylene-malonic acid diethyl ester

A mixture of aniline (25.6 g, 0.275 mol) and diethyl 2-(ethoxymethylene)malonate (62.4 g, 0.288 mol) was heated at 140-150° C. for 2 h. The mixture was cooled to room temperature and dried under reduced pressure to afford 2-phenylaminomethylene-malonic acid diethyl ester as a solid, which was used in the next step without further purification. ¹H NMR (DMSO-d6) δ 11.00 (d, 1H), 8.54 (d, J=13.6 Hz, 1H), 7.36-7.39 (m, 2H), 7.13-7.17 (m, 3H), 4.17-4.33 (m, 4H), 1.18-1.40 (m, 6H).

### Step 2: 4-Hydroxyquinoline-3-carboxylic acid ethyl ester

A 1 L three-necked flask fitted with a mechanical stirrer was charged with 2-phenylaminomethylene-malonic acid diethyl ester (26.3 g, 0.100 mol), polyphosphoric acid (270 g) and phosphoryl chloride (750 g). The mixture was heated to 70° C. and stirred for 4 h. The mixture was cooled to room temperature and filtered. The residue was treated with aqueous Na₂CO₃ solution, filtered, washed with water and dried. 4-Hydroxyquinoline-3-carboxylic acid ethyl ester was obtained as a pale brown solid (15.2 g, 70%). The crude product was used in next step without further purification.

### Step 3: 4-Oxo-1,4-dihydroquinoline-3-carboxylic acid

4-Hydroxyquinoline-3-carboxylic acid ethyl ester (15 g, 69 mmol) was suspended in sodium hydroxide solution (2N, 150 mL) and stirred for 2 h at reflux. After cooling, the mixture was filtered, and the filtrate was acidified to pH 4 with 2N HCl. The resulting precipitate was collected via filtration, washed with water and dried under vacuum to give 4-oxo-1,4-dihydroquinoline-3-carboxylic acid as a pale white solid (10.5 g, 92%). ¹H NMR (DMSO-d6) δ 15.34 (s, 1H), 13.42 (s, 1H), 8.89 (s, 1H), 8.28 (d, J=8.0 Hz, 1H), 7.88 (m, 1H), 7.81 (d, J=8.4 Hz, 1H), 7.60 (m, 1H).

### Part B: Synthesis of N-(2,4-di-tert-butyl-5-hydroxyphenyl)-4-oxo-1,4-dihydroquinoline-3-carboxamide

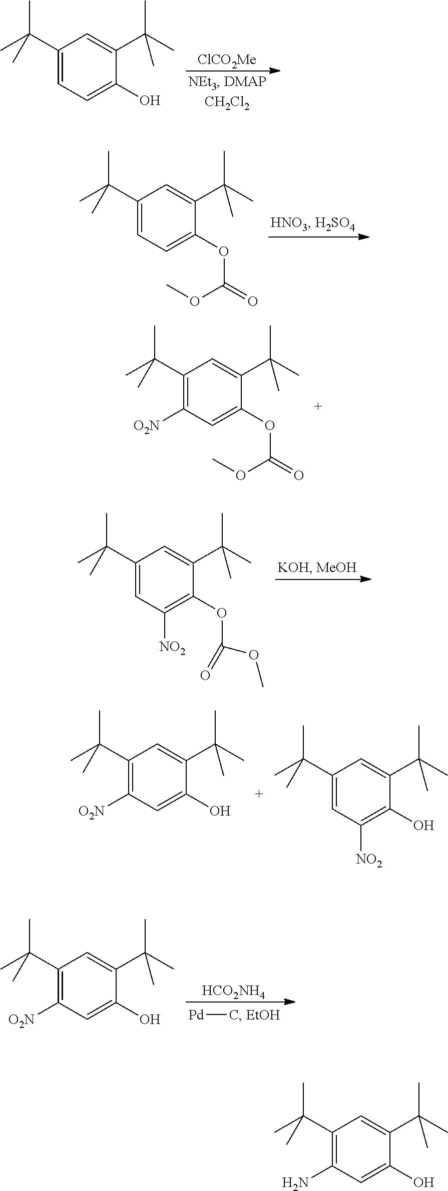

### Step 1: Carbonic acid 2,4-di-tert-butyl-phenyl ester methyl ester

Methyl chloroformate (58 mL, 750 mmol) was added dropwise to a solution of 2,4-di-tert-butyl-phenol (103.2 g, 500 mmol), Et₃N (139 mL, 1000 mmol) and DMAP (3.05 g, 25 mmol) in dichloromethane (400 mL) cooled in an ice-water bath to 0° C. The mixture was allowed to warm to room temperature while stirring overnight, then filtered through silica gel (approx. 1 L) using 10% ethyl acetate-hexanes (˜4 L) as the eluent. The combined filtrates were concentrated to yield carbonic acid 2,4-di-tert-butyl-phenyl ester methyl ester as a yellow oil (132 g, quant.). ¹H NMR (400 MHz, DMSO-d6) δ 7.35 (d, J=2.4 Hz, 1H), 7.29 (dd, J=8.5, 2.4 Hz, 1H), 7.06 (d, J=8.4 Hz, 1H), 3.85 (s, 3H), 1.30 (s, 9H), 1.29 (s, 9H).

### Step 2: Carbonic acid 2,4-di-tert-butyl-5-nitro-phenyl ester methyl ester and Carbonic acid 2,4-di-tert-butyl-6-nitro-phenyl ester methyl ester

To a stirring mixture of carbonic acid 2,4-di-tert-butyl-phenyl ester methyl ester (4.76 g, 180 mmol) in conc. sulfuric acid (2 mL), cooled in an ice-water bath, was added a cooled mixture of sulfuric acid (2 mL) and nitric acid (2 mL). The addition was done slowly so that the reaction temperature did not exceed 50° C. The reaction was allowed to stir for 2 h while warming to room temperature. The reaction mixture was then added to ice-water and extracted into diethyl ether. The ether layer was dried (MgSO₄), concentrated and purified by column chromatography (0-10% ethyl acetate-hexanes) to yield a mixture of carbonic acid 2,4-di-tert-butyl-5-nitro-phenyl ester methyl ester and carbonic acid 2,4-di-tert-butyl-6-nitro-phenyl ester methyl ester as a pale yellow solid (4.28 g), which was used directly in the next step.

### Step 3: 2,4-Di-tert-butyl-5-nitro-phenol and 2,4-Di-tert-butyl-6-nitro-phenol

The mixture of carbonic acid 2,4-di-tert-butyl-5-nitro-phenyl ester methyl ester and carbonic acid 2,4-di-tert-butyl-6-nitro-phenyl ester methyl ester (4.2 g, 14.0 mmol) was dissolved in MeOH (65 mL) before KOH (2.0 g, 36 mmol) was added. The mixture was stirred at room temperature for 2 h. The reaction mixture was then made acidic (pH 2-3) by adding conc. HCl and partitioned between water and diethyl ether. The ether layer was dried (MgSO₄), concentrated and purified by column chromatography (0-5% ethyl acetate-hexanes) to provide 2,4-di-tert-butyl-5-nitro-phenol (1.31 g, 29% over 2 steps) and 2,4-di-tert-butyl-6-nitro-phenol. 2,4-Di-tert-butyl-5-nitro-phenol: ¹H NMR (400 MHz, DMSO-d6) δ 10.14 (s, 1H, OH), 7.34 (s, 1H), 6.83 (s, 1H), 1.36 (s, 9H), 1.30 (s, 9H). 2,4-Di-tert-butyl-6-nitro-phenol: ¹H NMR (400 MHz, CDCl₃) δ 11.48 (s, 1H), 7.98 (d, J=2.5 Hz, 1H), 7.66 (d, J=2.4 Hz, 1H), 1.47 (s, 9H), 1.34 (s, 9H).

### Step 4: 5-Amino-2,4-di-tert-butyl-phenol

To a refluxing solution of 2,4-di-tert-butyl-5-nitro-phenol (1.86 g, 7.40 mmol) and ammonium formate (1.86 g) in ethanol (75 mL) was added Pd-5% wt. on activated carbon (900 mg). The reaction mixture was stirred at reflux for 2 h, cooled to room temperature and filtered through Celite. The Celite was washed with methanol and the combined filtrates were concentrated to yield 5-amino-2,4-di-tert-butyl-phenol as a grey solid (1.66 g, quant.). ¹H NMR (400 MHz, DMSO-d6) δ 8.64 (s, 1H, OH), 6.84 (s, 1H), 6.08 (s, 1H), 4.39 (s, 2H, NH₂), 1.27 (m, 18H); HPLC ret. time 2.72 min, 10-99% CH₃CN, 5 min run; ESI-MS 222.4 m/z [M+H]⁺.

### Step 5: N-(5-hydroxy-2,4-di-tert-butyl-phenyl)-4-oxo-1H-quinoline-3-carboxamide

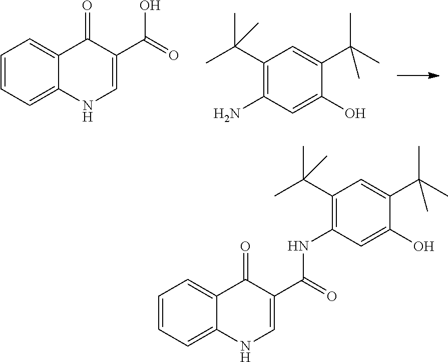

To a suspension of 4-oxo-1,4-dihydroquinolin-3-carboxylic acid (35.5 g, 188 mmol) and HBTU (85.7 g, 226 mmol) in DMF (280 mL) was added Et₃N (63.0 mL, 451 mmol) at ambient temperature. The mixture became homogeneous and was allowed to stir for 10 min before 5-amino-2,4-di-tert-butyl-phenol (50.0 g, 226 mmol) was added in small portions. The mixture was allowed to stir overnight at ambient temperature. The mixture became heterogeneous over the course of the reaction. After all of the acid was consumed (LC-MS analysis, MH+ 190, 1.71 min), the solvent was removed in vacuo. EtOH was added to the orange solid material to produce a slurry. The mixture was stirred on a rotovap (bath temperature 65° C.) for 15 min without placing the system under vacuum. The mixture was filtered and the captured solid was washed with hexanes to provide a white solid that was the EtOH crystalate. Et₂O was added to the solid obtained above until a slurry was formed. The mixture was stirred on a rotovapor (bath temperature 25° C.) for 15 min without placing the system under vacuum. The mixture was filtered and the solid captured. This procedure was performed a total of five times. The solid obtained after the fifth precipitation was placed under vacuum overnight to provide N-(5-hydroxy-2,4-di-tert-butyl-phenyl)-4-oxo-1H-quinoline-3-carboxamide as a white powdery solid (38 g, 52%). HPLC ret. time 3.45 min, 10-99% CH₃CN, 5 min run; ¹H NMR (400 MHz, DMSO-d6) δ 12.88 (s, 1H), 11.83 (s, 1H), 9.20 (s, 1H), 8.87 (s, 1H), 8.33 (dd, J=8.2, 1.0 Hz, 1H), 7.83-7.79 (m, 1H), 7.76 (d, J=7.7 Hz, 1H), 7.54-7.50 (m, 1H), 7.17 (s, 1H), 7.10 (s, 1H), 1.38 (s, 9H), 1.37 (s, 9H); ESI-MS m/z calc'd 392.21; found 393.3 [M+H]⁺.

### Example 4: Preparation of a Solid Dispersion Comprising Substantially Amorphous Compound I and HPMCAS-H Polymer

A solvent system of dichloromethane (DCM) and methanol (MeOH), is formulated according to the ratio 93.8 wt % DCM/6.2 wt % MeOH, in an appropriately sized container, equipped with a magnetic stirrer and stir plate. Into this solvent system, hypromellose acetate succinate polymer (HPMCAS, H grade) and Compound I were added according to the ratio 20 wt % hypromellose acetate succinate/80 wt % Compound I. The resulting mixture contained 15.0 wt % solids. The actual amounts of ingredients and solvents used to generate this mixture are recited in Table 14, below:

TABLE 14
Solid spray dispersion ingredients for amorphous Compound I.

| | Units | Batch |
|---|---|---|
| Compound I | g | 933.4 |
| HPMCAS | g | 233.3 |
| Total Solids | g | 1166.7 |
| DCM | g | 6201.4 |
| MeOH | g | 409.9 |
| Total Solvents | g | 6611.3 |
| Total Spray Solution Weight | g | 7778.0 |

The mixture was mixed until it was substantially homogenous and all components were substantially dissolved.

A spray drier, Anhydro MS-35 Spray Drier, fitted with two fluid 0.8 mm nozzle (Schlick series 970/0 S4), was used under normal spray drying mode, following the dry spray process parameters recited in Table 15.

TABLE 15
Spray drying dispersion processing parameters to generate
solid spray dispersion of amorphous Compound I.

| Parameter: | Value: | |
|---|---|---|
| Process Gas Flow Rate | 35 | Kg/hr |
| Nozzle Gas Flow Rate | 4.3 | Kg/hr |
| Feed Flow Rate | 2 | Kg/hr |
| Inlet Temperature | 71-74° | C. |
| Outlet Temperature | 42-44° | C. |
| Vacuum Dryer | 40° | C. |
| Temperature | | |
| Vacuum Drying Time | 24 | hours |

A high efficiency cyclone separated the wet product from the spray gas and solvent vapors. The wet product was transferred into trays and placed in vacuum dryer for drying to reduce residual solvents to a level of less than 3000 ppm for MeOH and less than 600 ppm of DCM and to generate dry spray dry dispersion of amorphous Compound I, containing <0.01% MeOH and <0.01% DCM.

### Example 5: Preparation of a Tablet Formulation

Screening/Weighing:

The solid dispersion comprising 80 wt % substantially amorphous Compound I and 20 wt % HPMCAS as shown in Example 4, the solid dispersion comprising 80 wt % substantially amorphous Compound II and 20 wt % HPMC (see PCT Publication No. WO 2015/160787, the entire contents are incorporated herein by reference), the solid dispersion comprising 80 wt % substantially amorphous Compound III, 19.5 wt % HPMCAS and 0.5 wt % sodium lauryl sulfate (see WO 2015/160787), and excipients (see Table 24) were screened prior to or after weigh-out. Screen sizes used were mesh #20 for all components except magnesium stearate which used mesh #60.

Blending:

The solid dispersion comprising substantially amorphous Compound I, the solid dispersion comprising substantially amorphous Compound II, and solid dispersion comprising substantially amorphous Compound III, and excipients were blended. The blending was performed using a bin blender. The components were blended for 5 minutes.

Dry Granulation:

The blend was granulated using a Gerteis roller compactor using combined smooth/knurled rolls and an integrated 1.0 mm mesh milling screen with pocketed rotor and paddle agitator. The roller compactor was operated with a roll gap of 2 mm, roll pressure of 4.4 kNcm, roll speed of 2 rpm, granulation speed of 80/80 (CW/CCW) rpm, and oscillation of 330/360 (CW/CCW)degrees.

Blending:

The roller compacted granules were blended with extra-granular excipients using a bin blender. The blending time was 5 minutes. Lubricant was added to bin and further blended for an additional 2 minutes.

Compression:

The compression blend was compressed into tablets using a Riva Piccola rotary tablet press. The weight of the tablets for a dose of 200 mg of substantially amorphous Compound I, 16.7 mg of substantially amorphous Compound II, and 100 mg of substantially amorphous Compound III was 565 mg.

Coating:

The core tablets were film coated using an Ohara tablet film coater. The film coat suspension was prepared by adding the coating material to purified water. The required amount of film coating suspension (3% of the tablet weight) was sprayed onto the tablets to achieve the desired weight gain.

TABLE 16
Tablet Comprising 200 mg Compound I, 16.7
mg Compound II and 100 mg Compound III.

| | | Amount per |
|---|---|---|
| | Ingredient | tablet (mg) |
| Intra-granular | Compound I SDD | 250 |
| | Compound II SDD | 20.9 |
| | Compound III SDD | 125 |
| | Croscarmellose Sodium | 33.9 |
| | Total | 429.8 |
| Extra-granular | Microcrystalline cellulose | 130 |
| | Magnesium Stearate | 5.6 |
| | Total | 135.6 |
| Total uncoated Tablet | | 565.4 |
| Film coat | Opadry | 17.0 |
| Total coated Tablet | | 582.4 |

### Example 6: Preparation of a Tablet Formulation

Screening/Weighing:

The solid dispersion comprising 80 wt % substantially amorphous Compound I and 20 wt % HPMCAS as shown in Example 4, the solid dispersion comprising 80 wt % substantially amorphous Compound II and 20 wt % HPMC (see WO 2015/160787, the entire contents are incorporated herein by reference), the solid dispersion comprising 80 wt % substantially amorphous Compound III, 19.5 wt % HPMCAS and 0.5 wt % sodium lauryl sulfate (see WO 2015/160787), and excipients (see Table 25) were screened prior to or after weigh-out. Screen sizes used were mesh #20 for all components except magnesium stearate which used mesh #60.

Dry Granulation 1:

The solid dispersions of Compounds I was granulated using a roller compactor. The blend was granulated using combined smooth/knurled rolls and with the integrated 1.0 mm mesh milling screen with pocketed rotor and paddle agitator. The roller compactor was operated with a roll gap of 2 mm, roll pressure of 4.4 kNcm, roll speed of 2 rpm,

Blending 2:

The solid dispersions of Compounds II, III, and excipients (see Table 25) were added to a blender and mixed to generate a blend.

Dry Granulation 2:

The blend was granulated using a roller compactor. The blend was granulated using combined smooth/knurled rolls and with the integrated 1.0 mm mesh milling screen with pocketed rotor and paddle agitator. The roller compactor was operated with a roll gap of 2 mm, roll pressure of 7.5 kNcm, and roll speed of 2 rpm,

Blending:

The roller compacted granules from dry granulation 1 and dry granulatuer 2 were blended with extra-granular excipients such as filler, disintegrant, and lubricant using a bin blender.

Compression & Coating:

The compression and coating were done in a similar manner as described in Example 5 using Thomas Flex 05 instead of an Ohara tablet film coater.

TABLE 17
Tablet Comprising 200 mg Compound 1, 16.7
mg Compound II and 100 mg Compound III.

| | | Amount per |
|---|---|---|
| | Ingredient | tablet (mg) |
| Intra-granular 1 | Compound I SDD | 250 |
| | Microcrystalline cellulose | 150 |
| | Croscarmellose Sodium | 16.7 |
| | Total | 416.7 |
| Intra-granular 2 | Compound II SDD | 20.9 |
| | Compound III SDD | 125 |
| | Microcrystalline cellulose | 36 |
| | Croscarmellose sodium | 12.7 |
| | Total | 194.6 |
| Extra-granular | Microcrystalline cellulose | 90.7 |
| | Croscarmellose sodium | 10.8 |
| | Magnesium Stearate | 7.2 |
| | Total | 108.7 |
| Total uncoated Tablet | | 720.0 |
| Film coat | Opadry | 21.6 |
| Total coated Tablet | | 741.6 |

### Example 6. Dissolution Studies of Solid Dispersions

The following solid dispersions have the following formulations:

The “Compound I mono SDD” is an 80:20% w/w ratio of Compound I:HPMCAS-HG;

The “Compound II mono SDD” is an 80:20% w/w ratio of Compound II:HPMC;

The “Compound III mono SDD” is an 80:19.5:0.5% w/w/w Compound III:HPMCAS:SLS;

The “Co-blend” is a 3:0.25:1.5 w/w/w ratio of Compound I mono SDD: Compound II mono SDD: Compound III mono SDD;

The “Co-SDD” is a 50.5:4.2:25.3:20% w/w/w/w ratio of Compound I: Compound II: Compound III:HPMC-E15.

Each of the following solid dispersions:

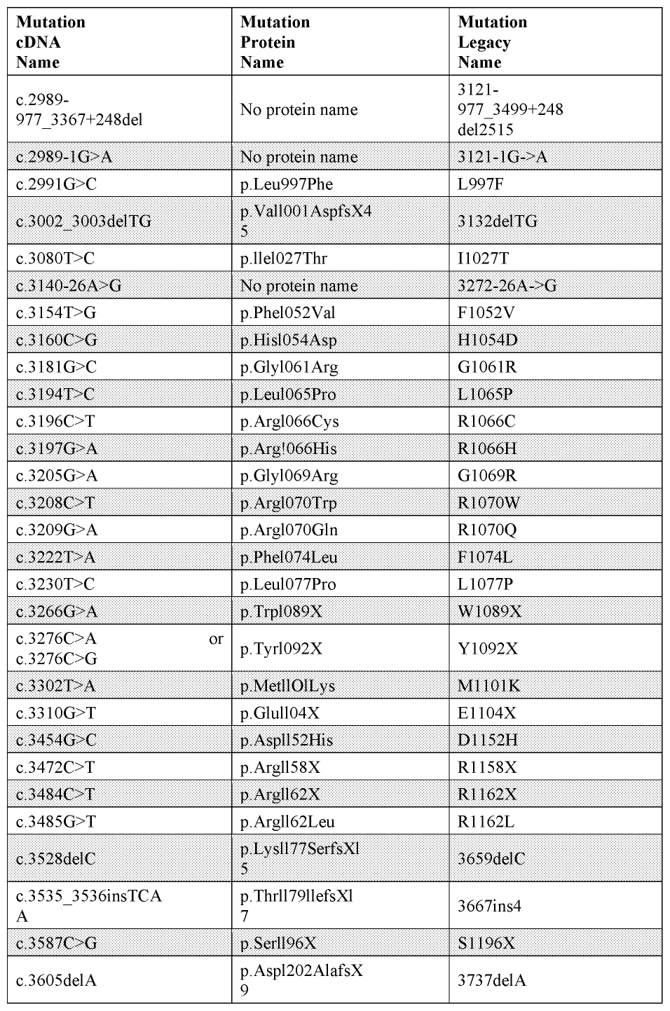

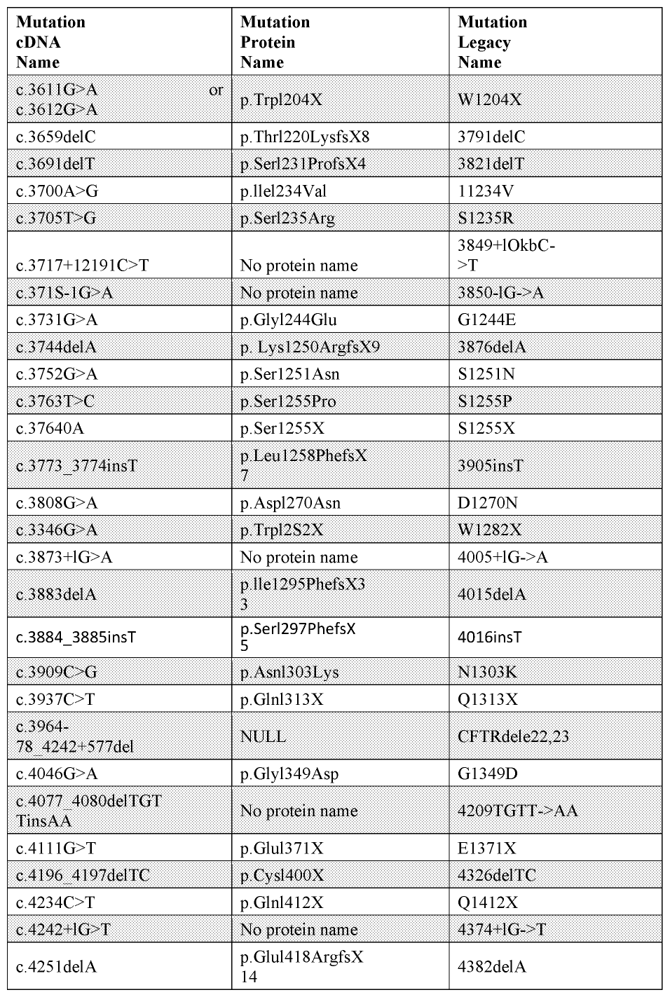

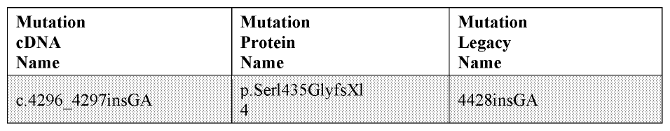

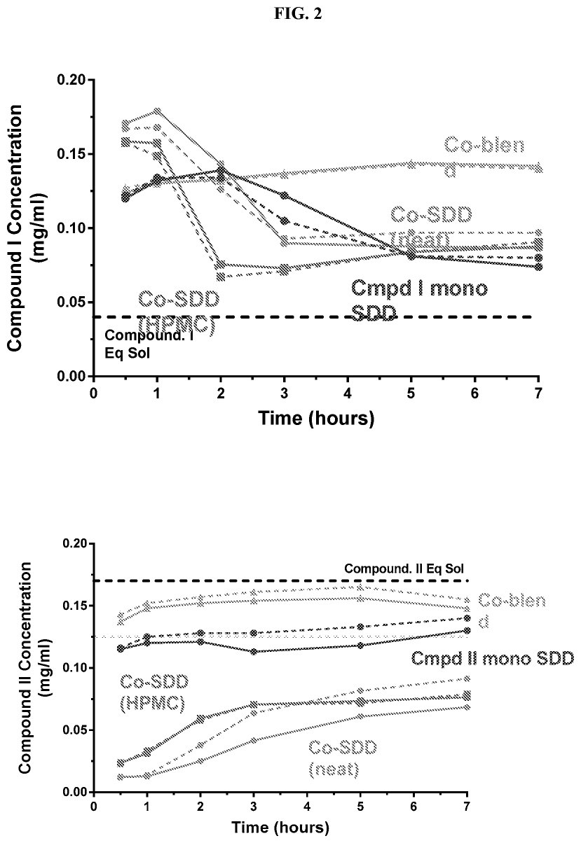

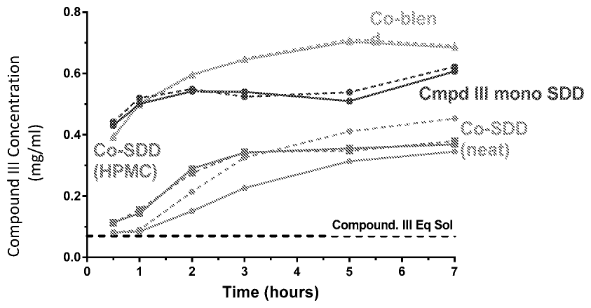

187.5 mg mono Compound I SDD
15.6 mg mono Compound II SDD
93.8 mg mono Compound III SDD
296.9 mg mono Compound I/II/III co-SDD (HPMC) blend
237.5 mg neat co-SDD
were added to 100 ml of simulated intestinal fluid, fed-state which was equilibrated at 37° C. and then stirred at 100 RPM for up to 24 hours. At 0.5, 1.0, 2.0, 3.0, 4.0, and 24 hours, 2.0 ml of sample was collected and centrifuged for 10 minutes. The supernatant (0.1 ml) was diluted to 1.0 ml with 80:20 acetonitrile:water and analyzed via reverse phase-HPLC (Poroshell 120 EC-C18 column, 120A, 2.7 μm, 3 mm×50 mm, 30° C., flow rate 1.0 mL/min, total run time 4.25 minutes, detection at 235 nm, isocratic gradient of 34:66 ratio of 0.1% trifluoroacetic acid in water: 0.1% trifluoroacetic acid in acetonitrile). Dissolution profiles are shown in FIG. 2.

Better kinetic solubility and physical stability of the co-blend SDDs over the co-SDD or the solid dispersion of any of Compound I, Compound II, or Compound III was observed. In the co-spray SDD, Compound I crystallized to Form A within 2 hours in this dissolution experiment. No crystallization was observed in the co-blend SDD.

### Example 7. Accelerated Stability Testing of Compound I Solid Dispersion

A solid dispersion of 50:50% w/w of Compound I:HPMCAS is chemically and physically stable for up to 12 months at 25° C./60% RH and 6 months at 40° C./75% RH in bulk packaging (Double LDPE bags, heat-sealed outer foil bag with 5% w/w molecular sieve desiccant). Under both conditions tested, crystalline Compound I was not observed at any time point throughout the duration of the study.

### Example 8. Precipitation Study of Compound I in Solutions of Fed State Simulated Intestinal Fluid with Polymer

A stock solution was prepared containing approximately 400 mg/ml of Compound I in dimethyl sulfoxide. Polymer solutions were prepared wherein each polymer as defined below was separately dissolved at both 0.1% and 1.0% by weight in 10 ml fed state simulated intestinal fluid. Twenty-five microliters of Compound I stock solution was added to each of the polymer/intestinal fluid solutions and allowed to mix at 37° C. At 10 min, 30 min, 60 min, 120 min, and 180 min, 0.5 ml was collected and filtered using a 0.45 micron PVDF centrifuge filter tube and spun at 8500 rpm for 5 min. The concentration of Compound I was quantitatively determined using HPLC enabled with a UV detector. The polymers PEG 3350, dimethylaminoethyl methacrylate-methylmethacrylate copolymer, PVP-K30, PVP-VA64, HPMC E15, Poloxamer 407, methyl cellulose and HPMCAS-H were generally shown to have higher concentrations of Compound I dissolved at various time points and concentrations relative to neat Compound I in simulated fluid.

### Example 9. Evaluation of Safety and Efficacy

A Phase 2, randomized, double-blind, placebo- and Compound II/Compound III-controlled, parallel-group, multicenter study was designed to evaluate the safety and efficacy of Compound I in dual and triple combination with Compound II and Compound III.

Part 1 of the study consisted of 2 cohorts: Cohort 1A and Cohort 1B. Part 2 consisted of 1 cohort. Subjects in Part 1 were ages 18 and older and heterozygous for the F508del mutation with a second CFTR allele carrying an minimal CFTR function mutation that is not expected to respond to Compound II/Compound III (F508del/MF) and subjects in Part 2 were ages 18 and older and were homozygous for F508del (F508del/F508del).

All parts of this study included a 4 week Treatment Period. Part 2 also included a 4-week Run-in Period and a 4-week Washout Period after the Treatment Period.

The Run-in Period was 4 weeks and was designed to establish a reliable on treatment Compound II/Compound III baseline for the Treatment Period. Subjects received Compound II 100 mg qd/Compound III 150 mg q12h during the Run-in Period. The first doses of Compound II and Compound III during the Run-in Period were administered at the Day −28 Visit and the last dose in the Run-in Period was administered on Day −1 (1 day before the Day 1 Visit).

For all Parts, to have been eligible to enter into the Treatment Period, after a screening period, subjects must have had stable CF disease and have remained on stable CF medication regimen during the 28 days before the Day 1 Visit and also must not have had an acute non-CF illness within 14 days before the Day 1 Visit.

The Treatment Period lasted 4 weeks. Drug administration details are provided below. The following definitions apply to the dosing regimens: “q12h” means every 12 hours; “qd” means once daily.

| | Treatment/ | Compound I | | Compound II | | Compound III | |
|---|---|---|---|---|---|---|---|
| Part | Control Arms | Dosage | | Dosage | | Dosage | |
| Part 1 | TC-1A | 200 | mg | 100 | mg qd | 150 | mg |
| | | q12h | | | | q12h | |
| Cohort 1A | Triple placebo | Placebo | | Placebo | | Placebo | |
| Part 1 | TC-1B-high | 600 | mg | 50 | mg | 300 | mg |
| | | q12h | | q12h | | q12h | |
| Cohort 1B | TC-1B-low | 200 | mg | 50 | mg | 150 | mg |
| | | q12h | | q12h | | q12h | |
| | Triple placebo | Placebo | | Placebo | | Placebo | |
| Part 2^{c} | TC-2 | 600 | mg | 50 | mg | 300 | mg |

| | q12h | q12h | | q12h | |
|---|---|---|---|---|---|
| Compound | Placebo^{b} | 100 | mg qd | 150 | mg |

| | |
|---|---|
| II/III | q12h |

In Part 2, the Washout Period after the Treatment Period lasted approximately 4 weeks and was designed to allow for the measurement of VX-440 off-treatment effects. Subjects received 100 mg qd Compound II/150 mg q12h Compound III during the Washout Period.

Primary objectives for the study were safety, tolerability and efficacy as assessed by mean absolute change in ppFEV₁ from baseline. Secondary endpoints included change in sweat chloride and Cystic Fibrosis Questionnaire-Revised (CFQ-R), among others.

In this Phase 2 study, women of childbearing potential were required to use pre-specified, non-hormonal methods of contraception based on results from previous preclinical reproductive toxicology studies.

Overall Safety Data: In the study, the triple combination regimen was generally well tolerated. The majority of adverse events were mild or moderate. The most common adverse event (>10%), regardless of treatment group, were infective pulmonary exacerbation, cough, sputum increased and diarrhea. There was one discontinuation due to an adverse event in the triple combination treatment groups (elevated liver enzymes>5× upper limit of normal in the Compound I 600 mg group) and one in the control groups (respiration abnormal and sputum increased). One additional patient treated with the triple combination had elevated liver enzymes (>8× upper limit of normal in the Compound I 600 mg group), which were observed on the final day of dosing. In both patients, the elevated liver enzymes returned to normal after treatment discontinuation or completion.

4-Week Efficacy Data in F508del/Min Patients

Part 1 of the study evaluated the triple combination for four weeks in 47 patients who have one F508del mutation and one minimal function mutation. A summary of the within-group lung function and sweat chloride data is provided below:

| Mean Absolute | Mean Absolute | Mean Absolute |
|---|---|---|
| Within-Group | Within-Group | Within-Group |
| Change From | Change in ppFEV₁ | Change in |
| Baseline Through | (percentage | Sweat Chloride |
| Day 29* | points) | (mmol/L) |
| Triple placebo | +1.4 | +1.6 |
| (n = 11) | (p = 0.4908) | (p = 0.6800) |
| TC-1A and TC-1B-low | +10.0 | −20.7 |
| (Compound I (200 mg | (p < 0.0001) | (p < 0.0001) |
| q12h) + Compound II | | |
| (50 mg q12h or 100 mg | | |
| QD) + Compound III | | |
| (150 mg q12h)) | | |
| (n = 18) | | |
| TC-1B-high | +12.0 | 33.1 |
| (Compound I (600 mg | (p < 0.0001) | (p < 0.0001) |
| q12h) + Compound II | | |
| (50 mg q12h) + | | |
| Compound III (300 mg | | |
| q12h)) (n = 18) | | |

*all p values are within group p-values based on mixed effect models; values expressed as ‘Through Day 29’ are the average of Day 15 and Day 29 measures

A secondary endpoint in the study measured mean absolute change in the Respiratory Domain of CFQ-R, a validated patient-reported outcome tool. In this study, the mean absolute improvement for patients with a minimal function mutation who received the triple combination were 18.3 points (Compound I 200 mg) and 20.7 points (Compound I 600 mg). The improvement for those who received placebo was 2.2 points.

4-Week Efficacy Data in F508del Homozygous Patients

Part 2 of the study evaluated the addition of Compound I for four weeks in 26 patients who have two copies of the F508del mutation, who were already receiving the combination of Compound II and Compound III. In this part of the study, all participants received four weeks of treatment with Compound II and Compound III and were then randomized to the addition of Compound I (n=20) or placebo (n=6) for four additional weeks. A summary of the within-group lung function and sweat chloride data for the triple combination treatment period, from baseline (end of the 4-week Compound II/Compound III run-in period), is provided below.

| Mean Absolute | Mean Absolute | Mean Absolute |
|---|---|---|
| Within-Group | Within-Group | Within-Group |
| Change From | Change in ppFEV₁ | Change in |
| Baseline Through | (percentage | Sweat Chloride |
| Day 29* | points) | (mmol/L) |
| Placebo + Compound II | −2.5 | +2.1 |
| (100 mg QD) + Compound II | (p = 0.2755) | (p = 0.7385) |
| (150 mg q12h) (n = 6) | | |
| TC-2 | +9.5 | −31.3 |
| (Compound I (600 mg q12h) + | (p < 0.0001) | (p < 0.0001) |
| Compound II (50 mg q12h) + | | |
| Compound III (300 mg q12h)) | | |
| (n = 20) | | |

*all p values are within group p-values based on mixed effect models; values expressed as ‘Through Day 29’ are the average of Day 15 and Day 29 measures

An overview of treatment emergent adverse events (“TEAE”) provided below.

| | | TC-2 | |
|---|---|---|---|
| | Compound | (Compound | |
| | II/ III | I/II/III) | Total |
| | N = 6 | N = 20 | N = 26 |
| | n (%) | n (%) | n (%) |
| Subjects with any | 5 (83.3) | 14 (70.0) | 19 (73.1) |
| TEAE | | | |
| Subjects with Severe | 1 (16.7) | 0 | 1 (3.8) |
| TEAE | | | |
| Subjects with life | 1 (16.7) | 0 | 1 (3.8) |
| threatening TEAE | | | |
| Subjects with Serious | 1 (16.7) | 1 (5.0) | 2 (7.7) |
| TEAE | | | |
| Subjects with TEAE | 0 | 1 (5.0) | 1 (3.8) |
| leading to treatment | | | |
| discontinuation | | | |
| Subjects with TEAE | 0 | 0 | 0 |
| leading to drug | | | |
| interruption | | | |

### OTHER EMBODIMENTS

The foregoing discussion discloses and describes merely exemplary embodiments of this disclosure. One skilled in the art will readily recognize from such discussion and from the accompanying drawings and claims, that various changes, modifications and variations can be made therein without departing from the spirit and scope of this disclosure as defined in the following claims.

1. A method of treating cystic fibrosis comprising administering to a patient in need thereof:
(A) 400 mg to 1600 mg of at least one compound chosen from Compound I: 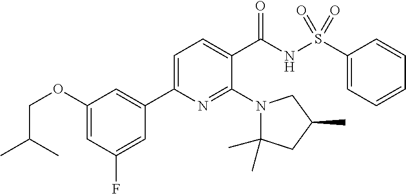 [SMILES: CC(C)COc1cc(F)cc(-c2ccc(C(=O)NS(=O)(=O)c3ccccc3)c(N3C[C@@H](C)CC3(C)C)n2)c1]
and pharmaceutically acceptable salts thereof daily; and
(B) 25 mg to 200 mg of at least one compound chosen from Compound II: 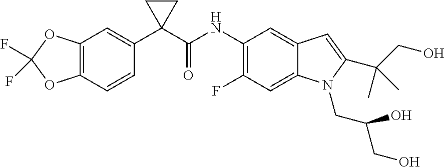 [SMILES: CC(C)(CO)c1cc2cc(NC(=O)C3(c4ccc5c(c4)OC(F)(F)O5)CC3)c(F)cc2n1C[C@@H](O)CO]
and pharmaceutically acceptable salts thereof daily; and
(C) 50 mg to 800 mg of at least one compound chosen from Compound III: 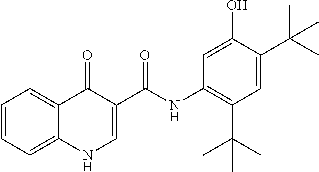 [SMILES: CC(C)(C)c1cc(C(C)(C)C)c(NC(=O)c2c[nH]c3ccccc3c2=O)cc1O]
and pharmaceutically acceptable salts thereof daily.

2.-33. (canceled)

34. The method of claim 1, wherein the Compound I or pharmaceutically acceptable salts thereof, Compound II or pharmaceutically acceptable salts thereof, and Compound III or pharmaceutically acceptable salts thereof, are administered in a pharmaceutical composition comprising:
(A) 200 mg to 1600 mg of at least one compound chosen from Compound and pharmaceutically acceptable salts thereof;
(B) 25 mg to 200 mg of at least one compound chosen from Compound 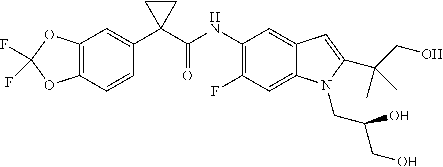 [SMILES: CC(C)(CO)c1cc2cc(NC(=O)C3(c4ccc5c(c4)OC(F)(F)O5)CC3)c(F)cc2n1C[C@@H](O)CO]
and pharmaceutically acceptable salts thereof;
(C) 50 mg to 800 mg of at least one compound chosen from Compound 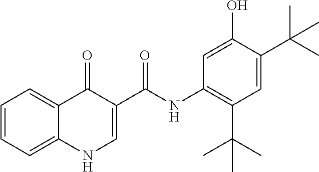 [SMILES: CC(C)(C)c1cc(C(C)(C)C)c(NC(=O)c2c[nH]c3ccccc3c2=O)cc1O]
and pharmaceutically acceptable salts thereof; and
(D) a pharmaceutically acceptable carrier.

35.-65. (canceled)

66. The method of claim 1, wherein:
(A) the Compound I or pharmaceutically acceptable salts thereof is administered in a first pharmaceutical composition comprising 200 mg to 1600 mg of at least one compound chosen from Compound I and pharmaceutically acceptable salts thereof and a pharmaceutically acceptable carrier; and
(B) the Compound II, or pharmaceutically acceptable salts thereof, and the Compound III, or pharmaceutically acceptable salts thereof are administered in a second pharmaceutical composition comprising: (i) 25 mg to 200 mg of at least one compound chosen from Compound II 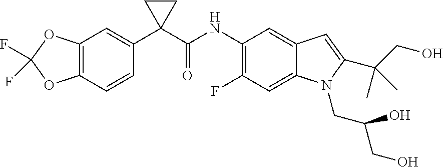 [SMILES: CC(C)(CO)c1cc2cc(NC(=O)C3(c4ccc5c(c4)OC(F)(F)O5)CC3)c(F)cc2n1C[C@@H](O)CO]
and pharmaceutically acceptable salts thereof (ii) 50 mg to 800 mg of at least one compound chosen from Compound III 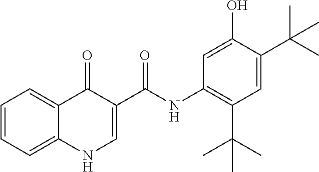 [SMILES: CC(C)(C)c1cc(C(C)(C)C)c(NC(=O)c2c[nH]c3ccccc3c2=O)cc1O]
and pharmaceutically acceptable salts thereof, and (iii) a pharmaceutically acceptable carrier.

67.-127. (canceled)

128. A single tablet comprising a first solid dispersion, a second solid dispersion, and a third solid dispersion,
(a) wherein the first solid dispersion comprises 50 mg to 800 mg of Compound I: 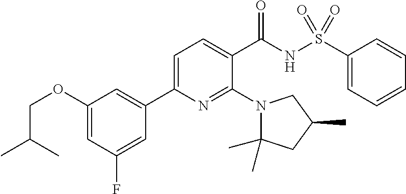 [SMILES: CC(C)COc1cc(F)cc(-c2ccc(C(=O)NS(=O)(=O)c3ccccc3)c(N3C[C@@H](C)CC3(C)C)n2)c1]
and 10 wt % to 60 wt % of a polymer relative to the total weight of the first solid dispersion;
(b) wherein the second solid dispersion comprises 3 mg to 70 mg of Compound II: 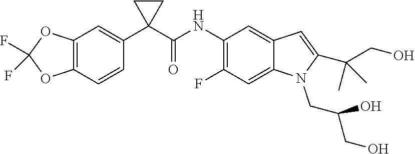 [SMILES: CC(C)(CO)c1cc2cc(NC(=O)C3(c4ccc5c(c4)OC(F)(F)O5)CC3)c(F)cc2n1C[C@@H](O)CO]
and 10 wt % to 30 wt % of a polymer relative to the total weight of the second solid dispersion; and
(c) wherein the third solid dispersion comprises 10 mg to 400 mg of Compound 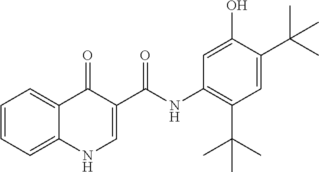 [SMILES: CC(C)(C)c1cc(C(C)(C)C)c(NC(=O)c2c[nH]c3ccccc3c2=O)cc1O]
and 10 wt % to 30 wt % of a polymer relative to the total weight of the third solid dispersion.

129.-164. (canceled)

165. The single tablet of claim 128, wherein the tablet comprises:
(a) 30 wt % to 50 wt % of the first solid dispersion comprising Compound I relative to the total weight of the tablet;
(b) 1 wt % to 8 wt % of the second solid dispersion comprising Compound II relative to the total weight of the tablet; and
(c) 10 wt % to 35 wt % of the third solid dispersion comprising Compound III relative to the total weight of the tablet;
wherein the first solid dispersion comprises 40 wt % to 90 wt % of Compound I and 10 wt % to 60 wt % of a polymer relative to the total weight of the first solid dispersion;
wherein the second solid dispersion comprises 70 wt % to 90 wt % of Compound II 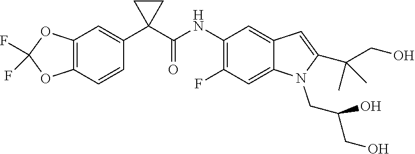 [SMILES: CC(C)(CO)c1cc2cc(NC(=O)C3(c4ccc5c(c4)OC(F)(F)O5)CC3)c(F)cc2n1C[C@@H](O)CO]
and 10 wt % to 30 wt % of a polymer relative to the total weight of the second solid dispersion; and
wherein the third solid dispersion comprises 70 wt % to 90 wt % of Compound III 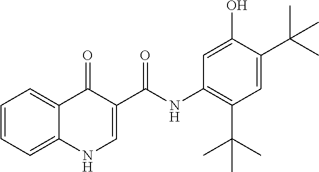 [SMILES: CC(C)(C)c1cc(C(C)(C)C)c(NC(=O)c2c[nH]c3ccccc3c2=O)cc1O]
and 10 wt % to 30 wt % of a polymer relative to the total weight of the third solid dispersion.

166.-196. (canceled)

197. A single tablet comprising a solid dispersion comprising 50 mg to 800 mg of Compound I: 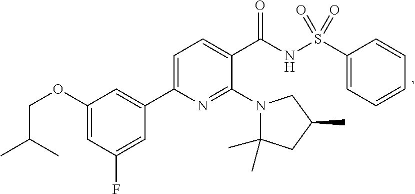 [SMILES: CC(C)COc1cc(F)cc(-c2ccc(C(=O)NS(=O)(=O)c3ccccc3)c(N3C[C@@H](C)CC3(C)C)n2)c1]
3 mg to 70 mg of Compound II: 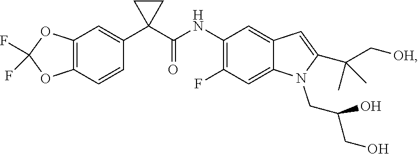 [SMILES: CC(C)(CO)c1cc2cc(NC(=O)C3(c4ccc5c(c4)OC(F)(F)O5)CC3)c(F)cc2n1C[C@@H](O)CO]
10 mg to 400 mg of Compound III: 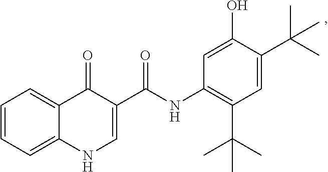 [SMILES: CC(C)(C)c1cc(C(C)(C)C)c(NC(=O)c2c[nH]c3ccccc3c2=O)cc1O]
and
one or more polymers.

198.-256. (canceled)

257. A method of treating cystic fibrosis in a patient comprising orally administering to the patient the single tablet of claim 128.

258.-265. (canceled)

266. A pharmaceutical composition comprising a solid dispersion comprising: (a) Compound I 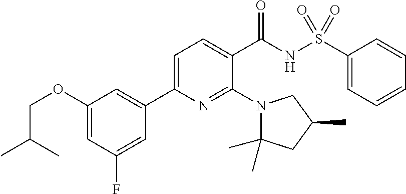 [SMILES: CC(C)COc1cc(F)cc(-c2ccc(C(=O)NS(=O)(=O)c3ccccc3)c(N3C[C@@H](C)CC3(C)C)n2)c1]
and (b) a polymer; and
a pharmaceutically acceptable carrier.

267.-284. (canceled)

285. The method of claim 1, wherein the method comprises administering:
(A) 200 mg of at least one compound chosen from Compound I and pharmaceutically acceptable salts thereof twice daily
(B) 100 mg of at least one compound chosen from Compound II and pharmaceutically acceptable salts thereof once daily 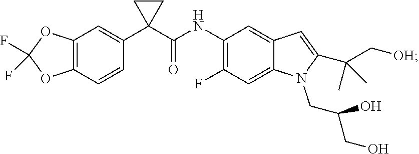 [SMILES: CC(C)(CO)c1cc2cc(NC(=O)C3(c4ccc5c(c4)OC(F)(F)O5)CC3)c(F)cc2n1C[C@@H](O)CO]
and
(C) 150 mg of at least one compound chosen from Compound III and pharmaceutically acceptable salts thereof twice daily

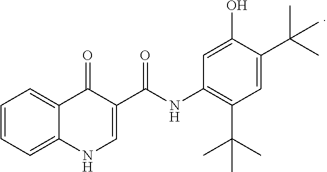 [SMILES: CC(C)(C)c1cc(C(C)(C)C)c(NC(=O)c2c[nH]c3ccccc3c2=O)cc1O]

286. The method of claim 1, wherein the method comprises administering:
(A) 200 mg of at least one compound chosen from Compound I and pharmaceutically acceptable salts thereof twice daily
(B) 50 mg of at least one compound chosen from Compound II and pharmaceutically acceptable salts thereof twice daily 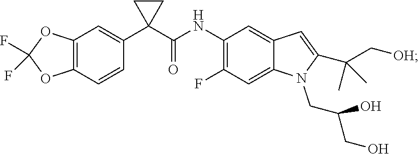 [SMILES: CC(C)(CO)c1cc2cc(NC(=O)C3(c4ccc5c(c4)OC(F)(F)O5)CC3)c(F)cc2n1C[C@@H](O)CO]
and
(C) 150 mg of at least one compound chosen from Compound III and pharmaceutically acceptable salts thereof twice daily

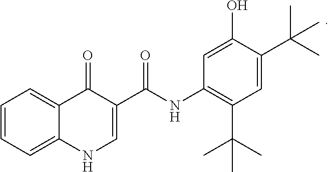 [SMILES: CC(C)(C)c1cc(C(C)(C)C)c(NC(=O)c2c[nH]c3ccccc3c2=O)cc1O]

287. The method of claim 1, wherein the method comprises administering:
(A) 600 mg of at least one compound chosen from Compound I and pharmaceutically acceptable salts thereof twice daily
(B) 50 mg of at least one compound chosen from Compound II and pharmaceutically acceptable salts thereof twice daily 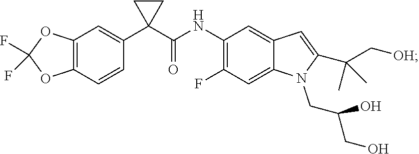 [SMILES: CC(C)(CO)c1cc2cc(NC(=O)C3(c4ccc5c(c4)OC(F)(F)O5)CC3)c(F)cc2n1C[C@@H](O)CO]
and
(C) 300 mg of at least one compound chosen from Compound III and pharmaceutically acceptable salts thereof twice daily

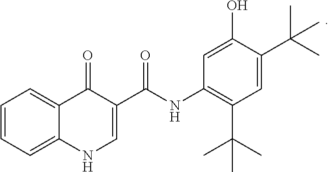 [SMILES: CC(C)(C)c1cc(C(C)(C)C)c(NC(=O)c2c[nH]c3ccccc3c2=O)cc1O]

288.-318. (canceled)
